(19) United States
(12) Patent Application Publication
(10) Pub. No.: US 2017/0327802 A1
(43) Pub. Date: Nov. 16, 2017

(54) Anti-Inflammatory Peptides and Composition Comprising the Same

(71) Applicants: Sang Jae KIM, Seoul (KR); GemVax & KAEL Co., Ltd., Seongnam-si (KR)

(72) Inventors: Sang Jae KIM, Seoul (KR); Kyung Hee KIM, Seongnam-si Gyeonggi-do (KR); Kyu-Yong LEE, Seongnam-si Gyeonggi-do (KR); Seong-Ho KOH, Seongnam-si Gyeonggi-do (KR); Bum Joon KIM, Seongnam-si (KR); Hyun-Hee PARK, Seongnam-si (KR); Sung Jin HUH, Seongnam-si (KR); Woo Jin LEE, Seongnam-si (KR); Hwain JANG, Seongnam-si (KR); Jung Soon HA, Seongnam-si (KR)

(21) Appl. No.: 15/664,806

(22) Filed: Jul. 31, 2017

Related U.S. Application Data
(63) Continuation-in-part of application No. 15/479,746, filed on Apr. 5, 2017.
Application No. 15/479,746 is a division of application No. 14/400,322, filed on Nov. 10, 2014.

(30) Foreign Application Priority Data
Jul. 20, 2012 (KR) 10-2012-0079096
Aug. 14, 2012 (KR) 10-2012-0089161
Aug. 14, 2012 (KR) 10-2012-0089162
Aug. 14, 2012 (KR) 10-2012-0089167
Sep. 19, 2012 (KR) 10-2012-0104144
Sep. 19, 2012 (KR) 10-2012-0104207

(51) Int. Cl. C12N 9/12; A23L 33/18; A61K 38/45

(52) U.S. Cl. CPC C12N 9/1276; A61K 38/45; A23L 33/18; A23V 2002/00; C12Y 207/07049

(57) ABSTRACT

The present invention relates to a peptide with anti-inflammatory activity, wherein the peptide comprises any one amino acid sequence of SEQ ID NO: 1 to SEQ ID NO: 161, the peptide has above 80% homology of amino acid sequence with above-mentioned sequences, or the peptide is the fragment of the above-mentioned peptides. The present invention also relates to an inflammatory composition comprising the above mentioned peptides. According to the present invention, a peptide that has at least one amino acid sequence of SEQ ID NO: 1 to SEQ ID NO: 161 has outstanding efficacy in both suppressing inflammation and in prophylactic means. Therefore, the composition comprising the peptides of this invention can be used as anti-inflammatory pharmaceutical compositions or as cosmetic compositions, in turn, treating and preventing a variety of different types of inflammatory diseases.

### CROSS-REFERENCE TO RELATED APPLICATIONS

This application is a continuation-in-part application of U.S. application Ser. No. 15/479,746, filed Apr. 5, 2017, which is a divisional application of U.S. application Ser. No. 14/400,322, with a National Stage entry date Nov. 10, 2014, which is the National Stage of International Application No. PCT/EP2013/055326, filed Mar. 15, 2013, which claims the priority benefit of Korean Patent Application Nos. 10-2012-0079096, filed Jul. 20, 2012; 10-2012-0089161, filed Aug. 14, 2012; 10-2012-0089162, filed Aug. 14, 2012; 10-2012-0089167, filed Aug. 14, 2012; 10-2012-0104144, filed Sep. 19, 2012; and 10-2012-0104207, filed Sep. 19, 2012, each of which is hereby incorporated by reference herein in its entirety.

### REFERENCE TO SEQUENCE LISTING

The Sequence Listing submitted herewith, as a text file named “2473_0740008_sequence listing.txt,” created on Jul. 21, 2017, and having a size of 63,344 bytes, is hereby incorporated by reference pursuant to 37 C.F.R. §1.52(e)(5).

### FIELD OF THE INVENTION

The present invention relates to anti-inflammatory peptides and compositions comprising the same.

### BACKGROUND OF THE INVENTION

Inflammation is a type of biological defense as a means of protecting the body from damage of biological tissues that could be caused by external physical stimuli, chemical stimuli such as exposure to various allergens, or invasion of microorganisms including bacteria, fungi and viruses.

The Cyclooxygenase (COX) pathway or Lipoxygenase (LOX) Pathway can be used for signaling inflammation, which produce prostaglandin, thromboxane, etc. Once the inflammatory signal is delivered, one of many changes that happen in the body is the expansion of the blood vessel for increased blood supply around the inflammation to concentrate blood cells such as neutrophils required for the inflammatory response. However, inflammatory diseases can result if an abnormal biological defense response occurs excessively. To prevent this, drugs that suppress excessive inflammatory responses by repressing enzymes used in inflammatory signaling pathways (for example COX-1, COX-2, 5-LOX, 12-LOX etc.) are under development.

According to response time, inflammation is categorized as acute inflammation (immediate response, non-specific response, several days to several weeks), chronic inflammation (delayed response, specific response, several weeks or more), subacute inflammation (a middle stage in between acute inflammation and chronic inflammation, characteristics of mixed product of mononuclear and polymorphounuclear). Also, aside from peptide factors, factors such as prostaglandin, leukotriene, lipid factors including platelet activating factor (PAF), synthetic enzyme of inflammation factor, free radical such as NO (nitric oxide), many kinds of cell adhesion molecules, the immune system, and coagulation factors can cause inflammation.

Once a cell is damaged due to the known causative agents of inflammation such as external biological factors (microbes, viruses, parasites), physical factors (mechanical stimuli, heat, radiation, electricity), and chemical factors, histamine and kinin are released. The released histamine and kinin will result in angiectasis, increased capillary permeability and concentration of macrophages at the inflammation site, and it causes increased blood flow rate, edema, immunocyte and antibody migration, pain and heat generation.

Currently used treatments for inflammation are synthetic drugs such as ibuprofen, antihistamines, steroids, cortisone, immunosuppressive agents, and immune agonist; those which only temporarily alleviate inflammation. These drugs do not fundamentally cure inflammation, and they have side effects such as hypersensitivity reaction, and deterioration of immune system,

Therefore, for effective alleviation of inflammation, research is being done to develop a substance that inhibits expression of the above mentioned inflammatory proteins. However, problems have arisen in anti-inflammation substances that had been developed previously. Diverse categories of anti-inflammatory drugs including Non-steroidal Anti-inflammatory Drugs (NSAIDs) and Steroidal Anti-inflammatory Drugs (SAIDs) have been developed; but not only do these drugs often bear side effects upon use, they also do not fundamentally cure the inflammation. Thus, there is a current need for anti-inflammatory drugs that are both physically and economically feasible. As one example, in acute or chronic inflammations such as chronic rheumatoid arthritis, not only do non-steroidal anti-inflammatory drugs suppress COX-2 enzyme activity, they are also known to suppress COX-1 activity, causing side effects such as gastrointestinal disorders.

The present invention was completed as the present inventors have found that peptides derived from telomerase can have anti-inflammatory properties.

Also, although low-molecular weight substances, nucleic acids, proteins, nano-particles, etc., have great potentials as therapeutic substances at a molecular level, their uses are limited due to the incompetence to penetrate into tissues and cell membrane. The development of a system to deliver such substances into the cell has been the active area of research over the last two decades. Transport the substances inside the cell has been a conversation topic in a treatment of molecular method. Low-molecular weight substances, nucleic acids or nano-particles were transported inside the cell by several reagents, electroporation or heatshock. However, it was difficult to find an adequate method of delivery of proteins inside the cell without disrupting the activity and integrity of proteins. In 1980s, in the research conducted on studying the cell penetrating capacity of HIV, it was found that HIV-TAT protein consisting of specific 11 amino acids play an important role in a process of transportation inside the cell. Thus, in 1990s, studies on finding the right method of transporting proteins inside the cell has been the intense area of research.

Telomere is known as a repetitive sequence of genetic material found at the ends of chromosomes that prevent chromosomes from damage or merging onto other chromosomes. The length of the telomere is shortened at each cell division, and after a certain number of cell division, the telomere length is extremely shortened to the extent in which the cell stops dividing and dies. On the other hand, the elongation of telomeres is known to extend the life span of a cell. For an example, cancer cells excrete an enzyme called telomerase, which prevents shortening of telomeres, thus resulting in proliferation of cancer cells.

Therefore the objective of this invention is to provide a novel peptide.

Another objective of present invention is to provide the polynucleotide that codes the novel peptide.

Another objective of present invention is to provide a peptide that has anti-inflammatory activity.

Another objective of present invention is to provide an anti-inflammatory composition that uses this peptide as an active ingredient.

Another objective of present invention is to provide a cosmetic composition that uses this peptide as an active ingredient.

Another objective of present invention is to provide a pharmaceutical composition that uses this peptide as an active ingredient.

Another objective of present invention is to provide a cell penetrating peptide.

Another objective of present invention is to provide a useful peptide as a carrier of the active ingredient in a cell.

Another objective of present invention is to provide a useful peptide as a carrier of the active ingredient in a cell, especially deliver to mitochondria locally.

Another objective of present invention is to provide a useful peptide for delivery of active ingredient to mitochondria for improvement, prophylaxis or treatment of mitochondria related disease or disorder.

Another objective of present invention is to provide a conjugate that an active ingredient and cell penetrating peptide are conjugated.

Another objective of present invention is to provide a composition comprising a conjugate of an active ingredient and cell penetrating peptide.

Another objective of present invention is to provide a pharmaceutical composition comprising a conjugate of an active ingredient and cell penetrating peptide.

Another objective of present invention is to provide a functional cosmetic composition comprising a conjugate of an active ingredient and cell penetrating peptide.

Another objective of present invention is to provide a health food composition comprising a conjugate of an active ingredient and cell penetrating peptide.

Another objective of present invention is to provide a contrast agent comprising a conjugate of an active ingredient and cell penetrating peptide.

### SUMMARY OF THE INVENTION

In one embodiment the present invention relates to a peptide with anti-inflammatory activity, wherein the peptide comprises at least one amino acid sequence of SEQ ID NO: 1 to SEQ ID NO: 161, or where the peptide has at least 80% sequence identity with the above-mentioned sequences, or the peptide is a fragment of the above-mentioned peptides.

In another embodiment, the above-mentioned fragment consists of 3 or more amino acids. For instance, the fragment may consist of 4, 5, 6, 7, 8, 9, 10, 11, 12, 13, 14, 15, 16, 17, 18, 19, 20, 21, 22, 23, 24, 25, or 26 amino acid residues.

In another embodiment, the above-mentioned peptide consists of 30 or less amino acids. For instance, the peptide may consist of 29, 28, 27, 26, 25, 24, 23, 22, 21, 20, 19, 18, 17, 16, 15, 14, 13, 12, 11, 10, 9, or 8 amino acid residues.

In another embodiment, the above-mentioned peptide consists of any one amino acid sequence of SEQ ID NO: 1 to SEQ ID NO: 161.

In another embodiment, the above-mentioned peptide comprises any one amino acid sequence selected from the group consisting of: SEQ ID NO: 1 to SEQ ID NO: 6, SEQ ID NO:9, SEQ ID NO: 11, SEQ ID NO: 14 to 21, SEQ ID NO: 23 to SEQ ID NO: 37, SEQ ID NO: 39 to SEQ ID NO: 44, SEQ ID NO: 47 to SEQ ID NO: 53, SEQ ID NO: 55 to SEQ ID NO: 61, SEQ ID NO: 63 to SEQ ID NO: 82, SEQ ID NO: 84 to SEQ ID NO: 94, SEQ ID NO: 96, SEQ ID NO: 99 to SEQ ID NO: 104, SEQ ID NO: 107 to SEQ ID NO: 109, SEQ ID NO: 115, SEQ ID NO: 116, SEQ ID NO: 120 to SEQ ID NO: 122, SEQ ID NO: 124, SEQ ID NO: 129 to SEQ ID NO: 133, SEQ ID NO: 142 to SEQ ID NO: 144, SEQ ID NO: 146, SEQ ID NO: 148, SEQ ID NO: 149, and SEQ ID NO: 155 to SEQ ID NO: 159.

In another embodiment, the above-mentioned peptide comprises any one amino acid sequence selected from the group consisting of: SEQ ID NO: 15 to SEQ ID NO: 18, SEQ ID NO: 23 to SEQ ID NO: 27, SEQ ID NO: 29, SEQ ID NO: 31 to SEQ ID NO: 34, SEQ ID NO: 39 to SEQ ID NO: 41, SEQ ID NO: 47, SEQ ID NO:48, SEQ ID NO: 51 to SEQ ID NO: 53, SEQ ID NO: 55 to SEQ ID NO: 58, SEQ ID NO: 61, SEQ ID NO: 65 to SEQ ID NO: 68, SEQ ID NO:70, SEQ ID NO: 73 to SEQ ID NO: 79, SEQ ID NO: 81, SEQ ID NO: 82, SEQ ID NO: 84 to SEQ ID NO: 87, SEQ ID NO: 90 to SEQ ID NO: 94, SEQ ID NO: 96, SEQ ID NO: 99, SEQ ID NO: 101 to SEQ ID NO: 104, SEQ ID NO: 107 to SEQ ID NO: 109, SEQ ID NO: 120, SEQ ID NO: 121, SEQ ID NO: 129 to SEQ ID NO: 132, SEQ ID NO: 142 to SEQ ID NO: 144, SEQ ID NO: 146, SEQ ID NO: 148, SEQ ID NO: 149, and SEQ ID NO: 157 to SEQ ID NO: 159.

In another embodiment, the above-mentioned peptide comprises any one amino acid sequence selected from the group consisting of: SEQ ID NO: 1 to SEQ ID NO: 5, SEQ ID NO: 7, SEQ ID NO: 9, SEQ ID NO: 10, SEQ ID NO: 12, SEQ ID NO: 13, SEQ ID NO: 15, SEQ ID NO: 17 to SEQ ID NO: 27, SEQ ID NO: 29, SEQ ID NO: 30, SEQ ID NO: 32 to SEQ ID NO: 53, SEQ ID NO: 55 to SEQ ID NO: 60, SEQ ID NO: 67, SEQ ID NO: 68, SEQ ID NO: 70, SEQ ID NO: 72 to SEQ ID NO: 82, SEQ ID NO: 84 to SEQ ID NO: 92, SEQ ID NO: 94, SEQ ID NO: 10 99 to SEQ ID NO: 112, SEQ ID NO: 114, SEQ ID NO: 127 to SEQ ID NO: 144, SEQ ID NO: 146, SEQ ID NO: 148, SEQ ID NO: 149, SEQ ID NO: 151 and SEQ ID NO: 153 to SEQ ID NO: 161.

In another embodiment, the above-mentioned peptide comprises any one amino acid sequence selected from the group consisting of: SEQ ID NO: 1 to SEQ ID NO: 5, SEQ ID NO: 7, SEQ ID NO: 9, SEQ ID NO: 10, SEQ ID NO: 12, SEQ ID NO: 13, SEQ ID NO: 15, SEQ ID NO: 17 to SEQ ID NO: 23, SEQ ID NO: 25 to SEQ ID NO: 27, SEQ ID NO: 29, SEQ ID NO: 30, SEQ ID NO: 33 to SEQ ID NO: 43, SEQ ID NO: 156, SEQ ID NO: 157 and SEQ ID NO: 159.

In another embodiment, the above-mentioned peptide comprises any one amino acid sequence selected from the group consisting of: SEQ ID NO: 1, SEQ ID NO: 2, SEQ ID NO: 4, SEQ ID NO: 6, SEQ ID NO: 7, SEQ ID NO: 8, SEQ ID NO: 9, SEQ ID NO: 10, SEQ ID NO: 12, SEQ ID NO: 14, SEQ ID NO: 15, SEQ ID NO: 17, SEQ ID NO: 18, SEQ ID NO: 20, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 44, SEQ ID NO: 45, SEQ ID NO: 52, SEQ ID NO: 57, SEQ ID NO: 60, SEQ ID NO: 61, SEQ ID NO: 62, SEQ ID NO: 64, SEQ ID NO: 65, SEQ ID NO: 67, SEQ ID NO: 68, SEQ ID NO: 69, SEQ ID NO: 91, SEQ ID NO: 99, SEQ ID NO: 100, SEQ ID NO: 104, SEQ ID NO: 106, SEQ ID NO: 107, SEQ ID NO: 108, SEQ ID NO: 109, SEQ ID NO: 111, SEQ ID NO: 112, SEQ ID NO: 115, SEQ ID NO: 117, SEQ ID NO: 118, SEQ ID NO: 119, SEQ ID NO: 120, SEQ ID NO: 122, SEQ ID NO: 124, SEQ ID NO: 125, SEQ ID NO: 126, SEQ ID NO: 129, SEQ ID NO: 130, SEQ ID NO: 131, SEQ ID NO: 132, SEQ ID NO: 135, SEQ ID NO: 146, SEQ ID NO: 151, SEQ ID NO: 154, and SEQ ID NO: 156.

In another embodiment, the above-mentioned peptide originates from human telomerase.

In one embodiment of the present invention, a polynucleotide encoding a peptide with anti-inflammatory activity, wherein the peptide comprises at least one amino acid sequence of SEQ ID NO: 1 to SEQ ID NO: 161, or the peptide has at least 80% sequence identity with the above-mentioned sequences, or the peptide is a fragment of the above-mentioned peptides, is provided.

In another embodiment of the polynucleotide, the above-mentioned peptide consists of 30 or less amino acids. For instance, the peptide may consist of 29, 28, 27, 26, 25, 24, 23, 22, 21, 20, 19, 18, 17, 16, 15, 14, 13, 12, 11, 10, 9, or 8 amino acid residues.

In another embodiment of the polynucleotide, the above-mentioned peptide consists of any one amino acid sequence of SEQ ID NO: 1 to SEQ ID NO: 161.

In another embodiment of the polynucleotide, the above-mentioned peptide originates from human telomerase.

In one embodiment of the present invention, anti-inflammatory composition comprising a peptide as active ingredient, wherein the peptide comprises at least one amino acid sequence of SEQ ID NO: 1 to SEQ ID NO: 161, the peptide has above 80% homology of amino acid sequence with above-mentioned sequences, or the peptide is a fragment of the abovementioned peptides, is provided.

In another embodiment of the composition, the above-mentioned peptide consists of 30 or less amino acid, cf. above.

In another embodiment of the composition, the above-mentioned peptide consists of any one amino acid sequence of SEQ ID NO: 1 to SEQ ID NO: 161. In another embodiment of the composition, the above-mentioned peptide originates from human telomerase.

In another embodiment of the composition, the above-mentioned composition is for treatment or prophylaxis of inflammatory disease.

In another embodiment of the composition, the above-mentioned composition is a cosmetic composition for improving or preventing skin inflammation.

In another embodiment of the composition, the above-mentioned composition is a pharmaceutical composition for treatment or prophylaxis of inflammatory diseases. In another embodiment of the composition, the above-mentioned composition is a food composition for treatment or prophylaxis of inflammation.

In another embodiment of the composition, the above-mentioned inflammatory disease is characterized by selecting from the group consisting of (1) general or localized inflammatory disease (for example, allergies; immune-complex disease; hayfever; hypersensitive shock; endotoxin shock; cachexia, hyperthermia; granulomatosis; or sarcoidosis); (2) gastrointestinal related diseases (for example, appendicitis; gastric ulcer; duodenal ulcer; peritonitis; pancreatitis; ulcerative, acute, or ischemic colitis; cholangitis; cholecystitis, steatorrhea, hepatitis, Crone's disease; or Whipple's Disease); (3) dermal related diseases (for example, psoriasis; burns; sunburns; dermatitis; Urticarial warts or wheal); (4) vascular related diseases (for example, angiitis; vasculitis; endocarditis; arteritis; atherosclerosis; thrombophlebitis; pericarditis; congestive heart failure; myocarditis; myocardial ischemia; periarteritis nodosa; recurrent stenosis; Buerger's disease; or rheumatic fever); (5) respiratory diseases (for example, asthma; epiglottitis; bronchitis; emphysema; rhinitis; cystic fibrosis; interstitial pneumonitis; COPD (chronic obstructive pulmonary disease); adult respiratory distress syndrome; coniosis; alveolitis; bronchiolitis; pharyngitis; pleurisy; or sinusitis); (6) bone, joint, muscle and connective tissue related diseases (for example, eosinophilic granuloma; arthritis; arthralgia; osteomyelitis; dermatomyositis; fasciitis; Paget's disease; gout; periodontal disease; rheumatoid arthritis; myasthenia gravis; ankylosing spondylitis; or synovitis); (7) urogenital disorders (for example, epididymitis; vaginitis; prostatitis; or urethritis); (8) central or peripheral nervous system related diseases (for example, Alzheimer's disease; meningitis; encephalitis; multiple sclerosis; cerebral infarction; cerebral embolism; Guillain-Barre syndrome; neuritis; neuralgia; spinal cord injury; paralysis; or uveitis); (9) virus (for example, influenza; respiratory syncytial virus; HIV; hepatitis B; hepatitis C; or herpes virus), infectious disease (for example, Dengue fever; or septicemia), fungal infection (for example, candidiasis); or bacterial, parasitic, and similar microbial infection (for example, disseminated bacteremia; malaria; onchocerciasis; or amebiasis); (10) autoimmune disease (for example, thyroiditis; lupus; Goodpasture's syndrome; allograft rejection; graft versus host disease; or diabetes); and (11) cancer or tumor disease (for example, Hodgkin's disease). In one embodiment of the present invention, a method for treating or preventing inflammatory diseases by administering the anti-inflammatory composition is provided.

In one embodiment of the present invention, a kit for prophylaxis or treatment of inflammatory diseases comprising: a peptide with anti-inflammatory activity or a composition comprising of the peptide, wherein the peptide comprises at least one amino acid sequence of SEQ ID NO: 1 to SEQ ID NO: 161, the peptide has above 80% homology with above-mentioned sequences, or the peptide is a fragment of the above-mentioned peptides; and instructions including at least one of administration dose, administration route, administration frequency, and indication of the peptide or composition, is provided.

In another embodiment, the disclosure provides a conjugate that may be a conjugate of cell penetrating carrier peptide and active ingredients, wherein the carrier peptide is the peptide comprising any one amino acid sequence of SEQ ID NO:71, SEQ ID NO:82, SEQ ID NO:96, SEQ ID NO:142, SEQ ID NO:153, and SEQ ID NO:164, the peptide having above 80% homology of amino acid sequence with above-mentioned sequences, or the fragment of the above-mentioned peptides, and wherein the peptide having above 80% homology of ammo acid sequence with above-mentioned sequences and the fragment of the same are the peptides that maintain cell penetrating ability of any one amino acid sequences of SEQ ID NO:71, SEQ ID NO:82, SEQ ID NO:96, SEQ ID NO:142, SEQ ID NO:153, and SEQ ID NO:164.

According to another embodiment of the conjugate in the present invention, the fragment may be made of 3 or more amino acids.

According to another embodiment of the conjugate in the present invention, the carrier peptide may be made of 30 or less amino acids.

According to another embodiment of the conjugate in the present invention, the above-mentioned carrier peptide may be the peptide having any one or more amino acid sequences of SEQ ID NO:71, SEQ ID NO:82, SEQ ID NO:96, SEQ ID NO:142, SEQ ID NO:153, and SEQ ID NO:164.

The contrast agent according to the one embodiment of the present invention may comprise any one conjugate above-mentioned.

The contrast agent according to the one embodiment of the present invention may be for contrasting a cell.

According to another embodiment of the contrast agent in the present invention, the cell may be a stem cell.

The composition according to one embodiment of the present invention may comprise any one of conjugates above-mentioned.

According to another embodiment of the composition in the present invention, the active ingredient may be for treatment or prevention of disease, and the composition may be pharmaceutical composition.

According to another embodiment of the composition in the present invention, the active ingredient may be the active ingredient for functional cosmetics, and the composition may be cosmetic composition.

According to another embodiment of the composition in the present invention, the active ingredient may be the active ingredient for functional health food, and the composition may be health food composition.

The cytoplasm targeting delivery system of active ingredient according to the one embodiment of the present invention may comprise any one of conjugates mentioned above, wherein the carrier peptide moves into a cytoplasm locally and performs a role of local cytoplasm delivering the mentioned active ingredients, wherein the peptide having above 80% homology of amino acid sequence with above-mentioned sequence and the fragment of the same are the peptides that maintain cell penetrating ability of any one amino acid sequences of SEQ ID NO:71, SEQ ID NO:82, SEQ ID NO:96, SEQ ID NO:142, SEQ ID NO:153, and SEQ ID NO:164.

According to another embodiment of the method in the present invention, the method may he for delivering the active ingredient locally into mitochondria inside a cell.

According to another embodiment of the cell penetrating peptide in the present invention, the above-mentioned carrier peptide may be the peptide having any one or more amino acid sequences of SEQ ID NO:71, SEQ ID NO:82, SEQ ID NO:96, SEQ ID NO:142, SEQ ID NO:153, and SEQ ID NO:164.

The polynucleotide according to the present invention may encode above-mentioned cell penetrating peptide.

The vector according to the present invention may comprise above-mentioned polynucleotide.

The transformed cell according to the present invention may comprise above-mentioned vector.

### INDUSTRIAL APPLICABILITY

According to the present invention, a peptide that has a sequence of SEQ ID NO: 1 to SEQ ID NO: 161 has outstanding efficacy in both suppressing inflammation and in prophylactic means. Therefore, the composition comprising the peptides of this invention can be used as anti-inflammatory pharmaceutical composition or as cosmetic composition, in turn, treating and preventing a variety of different types of inflammatory diseases.

Also, Active ingredients which are difficult to be transported inside a cell can be easily transported inside a cell by using the peptide, or the conjugate of the peptide and active ingredients, disclosed in the present invention. This means that efficacy of active ingredients can be increased and therefore the dosage can be lowered. As a result, side effects due to a drug administration can be minimized and effectiveness of treatment can be increased. Especially, as delivering drugs locally into mitochondria, mitochondria related diseases or disorders can be improved, and the effectiveness of prophylaxis and treatment of diseases can be increased. In a case of cosmetics, with a small amount of active ingredients, it can create an outstanding effect. By conjugating a peptide with a contrast substance, it can be used as a contrast substance to monitor a process of cell transplantation or transplanted cells in a cell treatment. Especially, it can be effectively used as a contrast substance for stem cells injected within a body.

### REFERENCES

KR2012-0130996A
KR2012-0133661A
KR2011-0060940A
US2011-0150873A1
Bonaldi T et al., EMBO J, (22)5551-60, 2003
Yankner B A eta/, Science (New York, N.Y.) [1990, 250(4978):279-282]
Dahlgren K N eta/, J. Bioi. Chem. 277:32046-32053, 2002.

### BRIEF DESCRIPTION OF THE DRAWINGS

FIG. 1 to FIG. 16 are results from screening TNF-α inhibition effects on monocytes.

FIG. 17 to FIG. 35 are results from screening TNF-α inhibition effects on cell line THP-1.

FIG. 36 to FIG. 108 are the western blot results of selected peptides which showed accumulation of HMGB1 in the cell.

FIG. 109 represents schematization of the reporter gene of SEQ ID NO:71 (pep73), SEQ ID NO:82 (pep84), SEQ ID NO:96 (pep98), SEQ ID NO:142 (pep144), SEQ ID NO:153 (pep155), and SEQ ID NO:164 (pep-RIA-148) is enhanced green fluorescent protein (enhanced green fluorescent protein, EGFP) fusion constructs produced method of recombinant DNA clones (clone).

FIG. 110A to FIG. 115B represent illustrating that the cellular uptake of the cell numbers from a HeLa cell treated with after the FITC fused to the peptide of SEQ ID NO:71 (pep73), SEQ ID NO:82 (pep84), SEQ ID NO:96 (pep98), SEQ ID NO:142 (pep144), SEQ ID NO:153 (pep155), and SEQ ID NO:164 (pep-RIA-148), and analyzed by flow cytometry (Flow cytometry). The control cells were treated only FITC.

FIG. 116 to FIG. 119 represents illustrating that the cellular uptake of the cell numbers from a Huh7 cell line treated with after the FITC fused to the peptide of SEQ ID NO:71 (pep73), SEQ ID NO:82 (pep84), SEQ ID NO:96 (pep98), SEQ ID NO:142 (pep144), SEQ ID NO:153 (pep155), and SEQ ID NO:164 (pep-RIA-148), and analyzed by flow cytometry (Flow cytometry). The control cells were treated only FITC.

FIG. 120 to FIG. 123 represents illustrating that the cellular uptake of the cell numbers from a human T lymphocyte cell line treated with after the FITC fused to the peptide of SEQ ID NO:71 (pep73), SEQ ID NO:82 (pep84), SEQ ID NO:96 (pep98), and SEQ ID NO:164 (pep-RIA-148), and analyzed by flow cytometry (Flow cytometry). The control cells were treated only FITC.

FIG. 124 to FIG. 129 represents illustrating that the result of toxicity and cell viability from a HeLa cell treated with after the FITC fused to the peptide of SEQ ID NO:71 (pep73), SEQ ID NO:82 (pep84), SEQ ID NO:96 (pep98), SEQ ID NO:142 (pep144), SEQ ID NO:153 (pep155), and SEQ ID NO:164 (pep-RIA-148), and analyzed by flow cytometry (Flow cytometry). The control cells were treated only FITC.

FIG. 130 to FIG. 135 represents illustrating that the cell penetrating of pep(CPP)-EGFP Fusion protein of SEQ ID NO:71 (pep73), SEQ ID NO:82 (pep84), SEQ ID NO:96 (pep98), SEQ ID NO:142 (pep144), SEQ ID NO:153 (pep155), and SEQ ID NO:164 (pep-RIA-148) analyzed by flow cytometry and confocal microscopy.

### DETAILED DESCRIPTION OF THE INVENTION

Since the present invention can have adaptability for diverse transformation and examples of practical application, below is a more detailed description of the present invention. Nevertheless, this is no means to limit the form of practical application; it should be understood that the intention is to include the concept and the extent of technology in all of the transformation, equivalents to alternatives. In describing the present invention, if any detailed description about the prior art is considered to deteriorate the fundamental principles of the present invention, the description will be omitted.

A telomere is known as a repetitive sequence of genetic material at the ends of chromosomes that prevent chromosomes from damage or merging of other chromosomes. The length of a telomere is shortened at each cell division, and after a certain number of cell division, the telomere length is extremely shortened to the extent in which the cell stops dividing and dies. On the other hand, the elongation of telomeres is known to extend the life span of a cell. For an example, cancer cells excrete an enzyme called telomerase, which prevents shortening of telomeres, thus resulting in proliferation of cancer cells. The present invention was accomplished upon the discovery of telomerase-derived peptides with anti-inflammatory effects.

In one embodiment of the present invention, a peptide with anti-inflammatory activities is provided. The peptide comprises at least one amino acid sequence of SEQ ID NO: 1 to SEQ ID NO: 161, the peptide has above 80% homology with above-mentioned sequences, or the peptide is a fragment of the above-mentioned peptides. The peptides described in SEQ ID NO: 1 to SEQ ID NO: 161 are as the following table 1. SEQ 25 ID NO: 162 lists the order of full length of human telomerase protein. SEQ ID NO: 163 lists the telomerase-derived peptide that consists of 16 amino acid sequence.

The “name” in Table 1 below was used for distinction of peptides. In a different specific embodiment of the present invention, more than one peptide of the mentioned peptides in SEQ ID NO: 1 to SEQ ID NO: 161 comprise a “synthetic peptide”, a synthesized peptide of selected areas of the telomerase. In the present specification, the term “pep” herein relates to a peptide that has any one of the SEQ ID NO: 1 to SEQ ID NO: 161, or, a peptide comprising of amino acid sequence above 80% homology with above-mentioned sequences, or a peptide fragment of above-mentioned peptides.

TABLE 1

| | | POSITION IN | | |
|---|---|---|---|---|
| SEQ ID NO | NAME | TELOMERASE | SEQUENCE | LENGTH |
| 1. | pep2 | [660-689] | ALFSVLNYERARRPGLLGASVLGLDDTHRA | 30 aa |
| 2. | pep3 | [663-677] | SVLNYERARRPGLLG | 15 aa |
| 3. | pep4 | [674-683] | GLLGASVLGL | 10 aa |
| 4. | pep5 | [615-624] | ALLTSRLRFI | 10 aa |
| 5. | pep6 | [613-621] | RPALLTSRL | 9 aa |
| 6. | pep7 | [653-661] | RLTSRVKAL | 9 aa |
| 7. | pep8 | [691-705] | RTFVLRVRAQDPPPE | 15 aa |
| 8. | pep9 | [653-667] | RLTSRVKALFSVLNY | 15 aa |
| 9. | pep10 | [651-665] | AERLTSRVKALFSVL | 15 aa |
| 10. | pep11 | [667-675] | YERARRPGL | 9 aa |
| 11. | pep12 | [675-683] | LLGASVLGL | 9 aa |
| 12. | pep13 | [680-689] | VLGLDDIHRA | 10 aa |
| 13. | pep14 | [677-686] | GASVLGLDDI | 10 aa |
| 14. | pep15 | [660-669] | ALFSVLNYER | 10 aa |
| 15. | pep16 | [663-672] | SVLNYERARR | 10 aa |
| 16. | pep17 | [679-688] | SVLGLDDIHR | 10 aa |
| 17. | pep18 | [662-671] | FSVLNYERAR | 10 aa |
| 18. | pep19 | [666-675] | NYERARRPGL | 10 aa |
| 19. | pep20 | [667-676] | YERARRPGLL | 10 aa |
| 20. | pep21 | [672-681] | RPGLLGASVL | 10 aa |
| 21. | pep22 | [668-676] | ERARRPGLL | 9 aa |
| 22. | pep23 | [680-688] | GLGLDDIHR | 9 aa |
| 23. | pep24 | [663-671] | SVLNYERAR | 9 aa |
| 24. | pep25 | [664-672] | VLNYERARR | 9 aa\| |
| 25. | pep26 | [670-678] | ARRPGLLGA | 9 aa |
| 26. | pep27 | [673-681] | PGLLGASVL | 9 aa |
| 27. | pep28 | [671-679] | RRPGLLGAS | 9 aa |
| 28. | pep29 | [660-668] | ALFSVLNYE | 9 aa |
| 29. | pep30 | [674-682] | GLLGASVLG | 9 aa |
| 30. | pep31 | [679-687] | SVLGLDDIH | 9 aa |
| 31. | pep32 | [668-675] | ERARRPGL | 8 aa |
| 32. | pep33 | [670-677] | ARRPGLLG | 8 aa |
| 33. | pep34 | [674-681] | GLLGASVL | 8 aa |
| 34. | pep35 | [669-676] | RARRPGLL | 8 aa |
| 35. | pep36 | [676-683[ | LGASVLGL | 8 aa |
| 36. | pep37 | [563-577] | VTETTFQKNRLFFYR | 15 aa |
| 37. | pep38 | [573-587] | LFFYRKSVWSKLQSI | 15 aa |
| 38. | pep39 | [583-597] | KLQSIGIRQHLKRVQ | 15 aa |
| 39. | pep40 | [603-617] | EAEVRQHREARPALL | 15 aa |
| 40. | pep41 | [613-627] | RPALLTSRLRFIPKP | 15 aa |
| 41. | pep42 | [623-637] | FIPKPDGLRPIVNMD | 15 aa |
| 42. | pep43 | [643-657] | RTFRREKRAERLTSR | 15 aa |
| 43. | pep45 | [683-697] | LDDIHRAWRTFVLRV | 15 aa |
| 44. | pep46 | [693-707] | FVLRVRAQDPPPELY | 15 aa |
| 45. | pep47 | [721-735] | PQDRLTEVIASIIKP | 15 aa |
| 46. | pep48 | [578-592] | KSVWSKLQSIGIRQH | 15 aa |
| 47. | pep49 | [593-608] | LKRVQLRELSEAEVRQ | 16 aa |
| 48. | pep50 | [1-20] | MPRAPRCRAVRSLLRSHYRE | 20 aa |
| 49. | pep51 | [21-40] | VLPLATFVRRLGPQGWRLVQ | 20 aa |
| 50. | pep52 | [41-60] | RGDPAAFRALVAQCLVCVPW | 20 aa |
| 51. | pep53 | [61-80] | DARPPPAAPSFRQVSCLKEL | 20 aa |
| 52. | pep54 | [81-100] | VARVLQRLCERGAKNVLAFG | 20 aa |
| 53. | pep55 | [101-120] | FALLDGARGGPPEAFTTSVR | 20 aa |
| 54. | pep56 | [121-140] | SYLPNTVTDALRGSSGAWGLL | 20 aa |
| 55. | pep57 | [141-160] | LRRVGDDVLVHLLARCALFV | 20 aa |
| 56. | pep58 | [161-180] | LVAPSCAYQVCGPPLYQLGA | 20 aa |
| 57. | pep59 | [181-200] | ATQARPPPHASGPRRRLGCE | 20 aa |
| 58. | pep60 | [201-220] | RAWNHSVREAGVPLGLPAPG | 20 aa |
| 59. | pep61 | [221-240] | ARRRGGSASRSLPLPKRPRR | 20 aa |
| 60. | pep62 | [241-260] | GAApepERTPVGQGSWAHPG | 20 aa |
| 61. | pep63 | [261-280] | RTRGPSDRGFCVVSPARPAE | 20 aa |
| 62. | pep64 | [281-300] | EATSLEGALSGTRHSHPSVG | 20 aa |
| 63. | pep65 | [301-320] | RQHHAGPPSTSRPPRPWDTP | 20 aa |
| 64. | pep66 | [321-340] | CPPVYAETKHFLYSSGDKEQ | 20 aa |
| 65. | pep67 | [341-360] | LRPSFLLSSLRPSLTGARRL | 20 aa |
| 66. | pep68 | [361-380] | VETIFLGSRPWMPGTPRRLP | 20 aa |
| 67. | pep69 | [381-400] | RLPQRYWQMRPLFLELLGNH | 20 aa |
| 68. | pep70 | [401-420] | AQCPYGVLLKTHCPLRAAVT | 20 aa |
| 69. | pep71 | [421-440] | PAAGVCAREKPQGSVAAPEE | 20 aa |
| 70. | pep72 | [441-460] | EDTDPRRLVQLLRQHSSPWQ | 20 aa |
| 71. | pep73 | [461-480] | VYGFVRACLRRLVPPGLWGS | 20 aa |
| 72. | pep74 | [481-500] | RHNERRFLRNTKKFISLGKH | 20 aa |
| 73. | pep75 | [501-520] | AKLSLQELTWKMSVRDCAWL | 20 aa |
| 74. | pep76 | [521-540] | RRSPGVGCVPAAEHRLREEI | 20 aa |
| 75. | pep77 | [541-560] | LAKFLHWLMSVYVVELLRSF | 20 aa |
| 76. | pep78 | [561-580] | FYVTETTFQKNRLFFYRKSV | 20 aa |
| 77. | pep79 | [581-600] | WSKLQSIGIRQHLKRVQLRE | 20 aa |
| 78. | pep80 | [601-620] | LSEAEVRQHREARPALLTSR | 20 aa |
| 79. | pep81 | [621-640] | LRFIPKPDGLRPIVNMDYVV | 20 aa |
| 80. | pep82 | [641-660] | GARTFRREKRAERLTSRVKA | 20 aa |
| 81. | pep83 | [661-680] | LFSVLNYERARRPGLLGASV | 20 aa |
| 82. | pep84 | [681-700] | LGLDDIHRAWRTFVLRVRAQ | 20 aa |
| 83. | pep85 | [701-720] | DPPPELYFVKVDVTGAYDTI | 20 aa |
| 84. | pep86 | [721-740] | PQDRLTEVIASIIKPQNTYC | 20 aa |
| 85. | pep87 | [741-760] | VRRYAVVQKAAHGHVRKAFK | 20 aa |
| 86. | pep88 | [761-780] | SHVSTLTDLQFYMRQFVAHL | 20 aa |
| 87. | pep89 | [781-800] | QETSPLRDAVVIEQSSSLNE | 20 aa |
| 88. | pep90 | [801-820] | ASSGLFDVFLRFMCHHAVRI | 20 aa |
| 89. | pep91 | [821-840] | RGKSYVQCQGIPQGSILSTL | 20 aa |
| 90. | pep92 | [841-860] | LCSLCYGDMENKLFAGIRRD | 20 aa |
| 91. | pep93 | [861-880] | GLLLRLVDDFLLVTPHLTHA | 20 aa |
| 92. | pep94 | [881-900] | KTFLRTLVRGVPEYGCVVNL | 20 aa |
| 93. | pep95 | [901-920] | RKTVVNFPVEDEALGGTAFV | 20 aa |
| 94. | pep96 | [921-940] | QMPAHGLFPWCGLLLDTRTL | 20 aa |
| 95. | pep97 | [941-960] | EVQSDYSSYARTSIRASLTF | 20 aa |
| 96. | pep98 | [961-980] | NRGFKAGRNMRRKLFGVLRL | 20 aa |
| 97. | pep99 | [981-1000] | KCHSLFLDLQVNSLQTVCTN | 20 aa |
| 98. | pep100 | [1001-1020] | IYKILLLQAYRFHACVLQLP | 20 aa |
| 99. | pep101 | [1021-1040] | FHQQVWKNPTFFLRVISDTA | 20 aa |
| 100. | pep102 | [1041-1060] | SLCYSILKAKNAGMSLGAKG | 20 aa |
| 101. | pep103 | [1061-1080] | AAGPLPSEAVQWLCHQAFLL | 20 aa |
| 102. | pep104 | [1081-1100] | KLTRHRVTYVPLLGSLRTAQ | 20 aa |
| 103. | pep105 | [1101-1120] | TQLSRKLPGTTLTALEAAAN | 20 aa |
| 104. | pep106 | [1121-1132] | PALPSDFKTILD | 20 aa |
| 105. | pep107 | [1-10] | MPRAPRCRAV | 20 aa |
| 106. | pep108 | [11-30] | RSLLRSHYREVLPLATFVRR | 20 aa |
| 107. | pep109 | [31-50] | LGPQGWRLVQRGDPAAFRAL | 20 aa |
| 108. | pep110 | [51-70] | VAQCLVCVPWDARPPPAAPS | 20 aa |
| 109. | pep111 | [71-90] | FRQVSCLKELVARVLQRLCE | 20 aa |
| 110. | pep112 | [91-110] | RGAKNVLAFGFALLDGARGG | 20 aa |
| 111. | pep113 | [111-130] | PPEAFTTSVRSYLPNTVTDA | 20 aa |
| 112. | pep114 | [131-150] | LRGSGAWGLLLRRVGDDVLV | 20 aa |
| 113. | pep115 | [151-170] | HLLARCALFVLVAPSCAYQV | 20 aa |
| 114. | pep116 | [171-190] | CGPPLYQLGAATQARPPPHA | 20 aa |
| 115. | pep117 | [191-210] | SGPRRRLGCERAWNHSVREA | 20 aa |
| 116. | pep118 | [211-230] | GVPLGLPAPGARRRGGSASR | 20 aa |
| 117. | pep119 | [231-250] | SLPLPKRPRRGAApepERTP | 20 aa |
| 118. | pep120 | [251-270] | VGQGSWAHPGRTRGPSDRGF | 20 aa |
| 119. | pep121 | [271-290] | CVVSPARPAEEATSLEGALS | 20 aa |
| 120. | pep122 | [291-310] | GTRHSHPSVGRQHHAGPPST | 20 aa |
| 121. | pep123 | [311-330] | SRPPRPWDTPCPPVYAETKH | 20 aa |
| 122. | pep124 | [331-350] | FLYSSGDKEQLRPSFLLSSL | 20 aa |
| 123. | pep125 | [351-370] | RPSLTGARRLVETIFLGSRP | 20 aa |
| 124. | pep126 | [371-390] | WMPGTPRRLPRLPQRYWQMR | 20 aa |
| 125. | pep127 | [391-410] | PLFLELLGNHAQCPYGVLLK | 20 aa |
| 126. | pep128 | [411-430] | THCPLRAAVTPAAGVCAREK | 20 aa |
| 127. | pep129 | [431-450] | PQGSVAAPEEEDTDPRRLVQ | 20 aa |
| 128. | pep130 | [451-470] | LLRQHSSPWQVYGFVRACLR | 20 aa |
| 129. | pep131 | [471-490] | RLVPPGLWGSRHNERRFLRN | 20 aa |
| 130. | pep132 | [491-510] | TKKFISLGKHAKLSLQELTW | 20 aa |
| 131. | pep133 | [511-530] | KMSVRDCAWLRRSPGVGCVP | 20 aa |
| 132. | pep134 | [531-550] | AAEHRLREEILAKFLHWLMS | 20 aa |
| 133. | pep135 | [551-570] | VYVVELLRSFFYVTETTFQK | 20 aa |
| 134. | pep136 | [571-590] | NRLFFYRKSVWSKLQSIGIR | 20 aa |
| 135. | pep137 | [591-610] | QHLKRVQLRELSEAEVRQHR | 20 aa |
| 136. | pep138 | [611-630] | EARPALLTSRLRFIPKPDGL | 20 aa |
| 137. | pep139 | [631-650] | RPIVNMDYVVGARTFRREKR | 20 aa |
| 138. | pep140 | [651-670] | AERLTSRVKALFSVLNYERA | 20 aa |
| 139. | pep141 | [671-690] | RRPGLLGASVLGLDDIHRAW | 20 aa |
| 140. | pep142 | [691-710] | RTFVLRVRAQDPPPELYFVK | 20 aa |
| 141. | pep143 | [711-730] | VDVTGAYDTIPQDRLTEVIA | 20 aa |
| 142. | pep144 | [731-750] | SIIKPQNTYCVRRYAVVQKA | 20 aa |
| 143. | pep145 | [751-770] | AHGHVRKAFKSHVSTLTDLQ | 20 aa |
| 144. | pep146 | [771-790] | PYMRQFVAHLQETSPLRDAV | 20 aa |
| 145. | pep147 | [791-810] | VIEQSSSLNEASSGLFDVFL | 20 aa |
| 146. | pep148 | [811-830] | RFMCMHAVRIRGKSYVQCQG | 20 aa |
| 147. | pep149 | [831-850] | IPQGSILSTLLCSLCYGDME | 20 aa |
| 148. | pep150 | [851-870] | NKLFAGIRRDGLLLRLVDDF | 20 aa |
| 149. | pep151 | [871-890] | LLVTPHLTHAKTFLRTLVRG | 20 aa |
| 150. | pep152 | [891-910] | VPEYGCVVNLRKTVVNFPVE | 20 aa |
| 151. | pep153 | [911-930] | DEALGGTAFVQMPAHGLFPW | 20 aa |
| 152. | pep154 | [931-950] | CGLLLDTRTLEVQSDYSSYA | 20 aa |
| 153. | pep155 | [951-970] | RTSIRASLTFNRGFKAGRNM | 20 aa |
| 154. | pep156 | [971-990] | RRKLFGVLRLKCHSLFLDLQ | 20 aa |
| 155. | pep157 | [991-1010] | VNSLQTVCTNIYKILLLQAY | 20 aa |
| 156. | pep158 | [1011-1030] | RFHACVLQLPFHQQVWKNPT | 20 aa |
| 157. | pep159 | [1031-1050] | FFLRVISDTASLCYSILKAK | 20 aa |
| 158. | pep160 | [1051-1070] | NAGMSLGAKGAAGPLPSEAV | 20 aa |
| 159. | pep161 | [1071-1090] | QWLCHQAFLLKLTRHRVTYV | 20 aa |
| 160. | pep162 | [1091-1110] | PLLGSLRTAQTQLSRKLPGT | 20 aa |
| 161. | pep163 | [1111-1132] | TLTALEAAANPALPSDFKTILD | 22 aa |
| 162. | Telomerase | [1-1132] | MPRAPRCRAVRSLLRSHYREVLPLATFVRR | 1132 aa |
| | | | LGPQGWRLVQRGDPAAFRALVAQCLVCVPW | |
| | | | DARPPPAAPSFRQVSCLKELVARVLQRLCERG | |
| | | | AKNVLAFGFALLDGARGGPPEAFTTSVRSYLP | |
| | | | NTVTDALRGSGAWGLLLRRVGDDVLVHLLAR | |
| | | | CALFVLVAPSCAYQVCGPPLYQLGAATQARPP | |
| | | | PHASGPRRRLGCERAWNHSVREAGVPLGLPA | |
| | | | PGARRRGGSASRSLPLPKRPRRGAApepERTP | |
| | | | VGQGSWAHPGRTRGPSDRGFCVVSPARPAE | |
| | | | EATSLEGALSGTRHSHPSVGRQHHAGPPSTS | |
| | | | RPPRPWDTPCPPVYAETKHFLYSSGDKEQLR | |
| | | | PSFLLSSLRPSLTGARRLVETIFLGSRPWMPG | |
| | | | TPRRLPRLPQRYWQMRPLFLELLGNHAQCPY | |
| | | | GVLLKTHCPLRAAVTPAAGVCAREKPQGSVA | |
| | | | APEEEDTDPRRLVQLLRQHSSPWQVYGFVRA | |
| | | | CLRRLVPPGLWGSRHNERRFLRNTKKFISLG | |
| | | | KHAKLSLQELTWKMSVRDCAWLRRSPGVGC | |
| | | | VPAAEHRLREEILAKFLHWLMSVYVVELLRSF | |
| | | | FYVTETTFQKNRLFFYRKSVWSKLQSIGIRQH | |
| | | | LKRVQLRELSEAEVRQHREARPALLTSRLRFI | |
| | | | PKPDGLRPIVNMDYVVGARTFRREKRAERLT | |
| | | | SRVKALFSVLNYERARRPGLLGASVLGLDDIH | |
| | | | RAWRTFVLRVRAQDPPPELYFVKVDVTGAYD | |
| | | | TIPQDRLTEVIASIIKPQNTYCVRRYAVVQKA | |
| | | | AHGHVRKAFKSHVSTLTDLQPYMRQFVAHLQ | |
| | | | ETSPLRDAVVIEQSSSLNEASSGLFDVFLRFM | |
| | | | CHHAVRIRGKSYVQCQGIPQGSILSTLLCSLC | |
| | | | YGDMENKLFAGIRRDGLLLRLVDDFLLVTPHL | |
| | | | THAKTFLRTLVRGVPEYGCVVNLRKTVVNFPV | |
| | | | EDEALGGTAFVQMPAHGLFPWCGLLLDTRTL | |
| | | | EVQSDYSSYARTSIRASLTFNRGFKAGRNMR | |
| | | | RKLFGVLRLKCHSLFLDLQVNSLQTVCTNIYK | |
| | | | ILLLQAYRFHACVLQLPFHQQVWKNPTFFLRV | |
| | | | ISDTASLCYSILKAKNAGMSLGAKGAAGPLPS | |
| | | | EAVQWLCHQAFLLKLTRHRVTYVPLLGSLRTA | |
| | | | QYQLSRKLPGTTLTALEAAANPALPSDFKTIL | |
| | | | D | |
| 163. | pep 1 | [611-626] | EARPALLTSRLRFIPK | 16 aa |

In one embodiment of the present invention, a polynucleotide that codes a peptide with anti-inflammatory activities is provided. The polynucleotide codes a peptide comprising at least one amino acid sequence of SEQ ID NO: 1 to SEQ ID NO: 161, a peptide having above 80% homology with above-mentioned sequences, or a peptide being a fragment of the above-mentioned peptides. The polynucleotide mentioned above enables production of the peptides in large quantities. For example, cultivation of vectors that include polynucleotides encoding peptides allows production of peptides in large quantities.

The peptides disclosed herein can include a peptide comprising amino acid sequence above 80%, above 85%, above 90%, above 95%, above 96%, above 97%, above 98%, or above 99% homology. Moreover, the peptides disclosed in the present invention can include a peptide comprising SEQ ID NO: 1 or its fragments, and a peptide with more than 1 5 transformed amino acid, more than 2 transformed amino acid, more than 3 transformed amino acid, more than 4 transformed amino acid, more than 5 transformed amino acid, more than 6 transformed amino acid, or more than 7 transformed amino acid. In the present specification and claims, the terms “homology” and “sequence identity” are used interchangeably to indicate the degree of sequence overlap between two amino acid (or if relevant: nucleic acid) sequences.

Unless otherwise stated the term “Sequence identity” for peptides as used herein refers to the sequence identity calculated as (n_{ref}−n_{dif})·100/n_{ref}, wherein n_{dif} is the total number of non-identical residues in the two sequences when aligned so that a maximum number of amino acids are identical and wherein nref is the number of residues in the shortest of the sequences. Hence, the DNA sequence agtcagtc will have a sequence identity of 75% with the sequence aatcaatc (n_{dif}=2 and n_{ref}=8).

In some embodiments the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (GAP, BESTFIT, FAST A, and TFASTA in the Wisconsin Genetics Software Package, Genetics Computer Group). The BLAST algorithm (Altschul et al., 1990, Mol. Bioi. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

In one embodiment of the present invention, changes in amino acid sequence belong to the modification of peptide's physical and chemical characteristics. For example, amino acid transformation can be performed by improving thermal stability of the peptide, altering substrate specificity, and changing the optimal pH.

In one embodiment of the present invention, a peptide comprising amino acid sequence of at least one of SEQ ID NO: 1 to SEQ ID NO: 161, a peptide comprising of amino acid sequence above 80% homology with above-mentioned sequences, or a peptide fragment of abovementioned peptides is preferably made of 30 or less amino acids.

In one embodiment of the present invention, a peptide comprising amino acid sequence of at least one of SEQ ID NO: 1 to SEQ ID NO: 161, a peptide comprising of amino acid sequence above 80% homology with above-mentioned sequences, or a peptide fragment of abovementioned peptides comprises a peptide originates from telomerase, more specifically, telomerase of Homo sapiens.]

The term “amino acid” herein includes not only the 22 standard amino acids that are naturally integrated into peptide but also the 0-isomers and transformed amino acids. Therefore, in a specific embodiment of the present invention, a peptide herein includes a peptide having 0-amino acids. On the other hand, a peptide may include non-standard amino acids such as those that have been post-translationally modified. Examples of posttranslational modification include phosphorylation, glycosylation, acylation (including acetylation, myristorylation, plamitoylation), alkylation, carboxylation, hydroxylation, glycation, biotinylation, ubiquitinylation, transformation in chemical properties (e.g. 13-removing deimidation, deamidation) and structural transformation (e.g. formation of disulfide bridge). Also, changes of amino acids are included and the changes of amino acids occur due to chemical reaction during the combination process with crosslinkers for formation of a peptide conjugate.

A peptide disclosed herein may be a wild-type peptide that has been identified and isolated from natural sources. On the other hand, when compared to peptide fragments of SEQ ID 20 NO: 1, the peptides disclosed herein may be artificial mutants that comprise one or more substituted, deleted and/or inserted amino acids. Amino acid alteration in wild-type polypeptide—not only in artificial mutants—comprises conservative substitution of amino acids that do not influence protein folding and or activation. Examples of conservative substitution belong to the group consisting of basic amino acids (arginine, lysine and histidine), acidic amino acids (glutamic acid and aspartic acid), polar amino acids (glutamine and asparagines), hydrophobic amino acids (leucine, isoleucine, valine and methionine), aromatic amino acids (phenylalanine, tryptophan and tyrosine), and small amino acids (glycine, alanine, serine, and threonine). The amino acid substitutions that do not generally alter the specific activity are known in the art of the present invention. Most common occurred alteration are Ala/Ser, Vai/IIe, Asp/Giu, Thr/Ser, Ala/Giy, Ala/Thr, Ser/Asn, Ala/Val, Ser/Giy, Tyr/Phe, Ala/Pro, Lys/Arg, Asp/Asn, Leu/IIe, Leu/Val, Ala/Giu, Asp/Giy, and the opposite alterations. Another example of conservative substitutions are shown in the following table 2.

TABLE 2

| | | Preferable |
|---|---|---|
| | Examples of residue | residue |
| Original amino acid | substitution | substitution |
| Ala (A) | val; leu; ile | Val |
| Arg (R) | lys; gin; asn | Lys |
| Asn (N) | gin; his; asp, lys; arg | Gin |
| Asp (D) | glu; asn | Glu |
| Cys (C) | ser; ala | Ser |
| Gin (Q) | asn; glu | Asn |
| Glu (E) | asp; gin | Asp |
| Gly (G) | ala | Ala |
| His (H) | asn; gin; lys; arg | Arg |
| Ile (I) | leu; val; met; ala; phe; | Leu |
| Leu (L) | norleucine; ile; val; met; ala; | lie |
| Lys (K) | arg; gin; asn | Arg |
| Met (M) | leu; phe; ile | Leu |
| Phe (F) | leu; val; ile; ala; tyr | Tyr |
| Pro (P) | ala | Ala |
| Ser(S) | thr | Thr |
| Thr (T) | ser | Ser |
| Trp (W) | tyr; phe | Tyr |
| Tyr (Y) | trp; phe; thr; ser | Phe |
| Val (V) | ile; leu; met; phe; ala; | Leu |

The substantial transformation of the biological properties of peptides are performed by selecting significantly different substitution in the following efficacies: (a) the efficacy in maintaining the structure of the polypeptide backbone in the area of substitution, such as sheet or helical three-dimensional structures, (b) the efficacy in maintaining electrical charge or hydrophobicity of the molecule in the target area, or (c) the efficacy of maintaining the bulk of the side chain. Natural residues are divided into groups by general side chain properties as the following:

(1) hydrophobicity: Norleucine, met, ala, val, leu, ile;

(2) neutral hydrophilicity: cys, ser, thr;

(3) acidity: asp, glu;

(4) basicity: asn, gin, his, lys arg;

(5) residue that affects chain orientation: gly, pro; and

(6) aromaticity: trp, tyr, phe.

Non-conservative substitutions may be performed by exchanging a member of the above classes to that of a different class. Any cysteine residues that are not related in maintaining the proper three-dimensional structure of the peptide can typically be substituted into serine, thus increasing the oxidative stability of the molecule and preventing improper cross-linkage. Conversely, improvement of stability can be achieved by adding cysteine bond(s) to the peptide.

Altered types of amino acids variants of peptides are those that antibody glycosylation pattern changed. The term “change” herein relates to deletion of carbohydrate residues and/or addition of at least one glycosylated residues that do not exist within a peptide. Glycosylation in peptides are typically N-connected or 0-connected. The term “N-connected” herein relates to that carbohydrate residues are attached to the side chain of asparagine residues. As tripeptide sequences, asparagine-X-serine and asparagine-X-threonine (where the X is any amino acid except proline) are the recognition sequence for attaching carbohydrate residue enzymatically to the side chain of asparagine. Therefore, with the presence of one of these tripeptide sequences in a polypeptide, the potential glycosylation sites are created. “0-connected glycosylation” means attaching one of sugar N-acetylgalactosamine, galactose, or xylose to hydroxyl amino acids. The hydroxyl amino acids are most typically serine or threonine, but 5-hydroxyproline or 5-hydroxylysine can be used.

Addition of glycosylation site to a peptide is conveniently performed by changing amino acid sequence to contain tripeptide sequence mentioned above (for N-linked glycosylation sites). These changes may be made by addition of at least one serine or theronine residues to the first antibody sequence, or by substitution with those residues (for O-linked glycosylation sites).

In one embodiment of the present invention, a polynucleotide is a nucleic acid molecule that can be spontaneous or artificial DNA or RNA molecules, either single-stranded or double-stranded.

The nucleic acid molecule can be one or more nucleic acids of same type (for example, having a same nucleotide sequence) or nucleic acids of different types. The nucleic acid molecules comprise one or more DNA, cDNA, decoy DNA, RNA, siRNA, miRNA shRNA, stRNA, snoRNA, snRNA PNA, antisense oligomer, plasmid and other modified nucleic acids, but not limited to those.

A HMGB1 protein is known as a cytokine. It first undergoes acetylation and translocation to cytoplasm by external stimulation. Then it is secreted out of the cell, therefore serving the role of inflammation-causing cytokine. Because when one has an inflammation due to such activity, HMGB1 protein is secreted out of the cell, and patients with inflammatory diseases such as Churg Strauss syndrome, rheumatoid arthritis and Sjogren's syndrome will present with elevated serum levels of HMGB1. Hence, if nucleus contains large amount of HMGB1 even when there is a stimulus that causes inflammation, it is suggestive of the fact that HMGB1 is not being secreted out of the cell, which means inflammation is being suppressed.

In one embodiment of the present invention, when treated a cell with a peptide comprising any one amino acid sequence of SEQ ID NO: 1 to SEQ ID NO: 161, a peptide having above 80% homology of amino acid sequence with above-mentioned sequences, or a fragment of the above-mentioned peptides, amount of HMGB1 within the nucleus increases. This represents that the peptides mentioned above have excellent inflammation preventive or suppressive effects.

Also, in specific embodiments of the present invention, a peptide comprising any one amino acid sequence of SEQ ID NO: 1 to SEQ ID NO: 161, a peptide having above 80% homology of amino acid sequence with above-mentioned sequences, or a fragment of the above-mentioned peptides, has an advantage in that it has high feasibility due to its low toxicity within a cell.

In the present invention, an “inflammatory disease” is a broad indication that refers to any disease that designates inflammation as a main cause or inflammation caused by disease. Specifically, an inflammatory disease includes (1) general or localized inflammatory disease (for example, allergies; immune-complex disease; hayfever; hypersensitive shock; endotoxin shock; cachexia, hyperthermia; granulomatosis; or sarcoidosis); (2) gastro-intestinal related diseases (for example, appendicitis; gastric ulcer; duodenal ulcer; peritonitis; pancreatitis; ulcerative, acute, or ischemic colitis; cholangitis; cholecystitis, steatorrhea, hepatitis, Crone's disease; or Whipple's Disease); (3) dermal related diseases (for example, psoriasis; burns; sunburns; dermatitis; Urticarial warts or wheal); (4) vascular related diseases (for example angiitis; vasculitis; endocarditis; arteritis; atherosclerosis; thrombophlebitis; pericarditis; congestive heart failure; myocarditis; myocardial ischemia; periarteritis nodosa; recurrent stenosis; Buerger's disease; or rheumatic fever); (5) respiratory diseases (for example, asthma; epiglottitis; bronchitis; emphysema; rhinitis; cystic fibrosis; interstitial pneumonitis; COPD (chronic obstructive pulmonary disease); adult respiratory distress syndrome; coniosis; alveolitis; bronchiolitis; pharyngitis; pleurisy; or sinusitis); (6) bone, joint, muscle and connective tissue related diseases (for example, eosinophilic granuloma; arthritis; arthralgia; osteomyelitis; dermatomyositis; fasciitis; Paget's disease; gout; periodontal disease; rheumatoid arthritis; myasthenia gravis; ankylosing spondylitis; or synovitis); (7) urogenital disorders (for example, epididymitis; vaginitis; prostatitis; or urethritis); (8) central or peripheral nervous system related diseases (for example, Alzheimer's disease; meningitis; encephalitis; multiple sclerosis; cerebral infarction; cerebral embolism; Guillain-Barre syndrome; neuritis; neuralgia; spinal cord injury; paralysis; or uveitis); (9) virus (for example, influenza; respiratory syncytial virus; HIV; hepatitis B; hepatitis C; or herpes virus), infectious disease (for example, Dengue fever; or septicemia), fungal infection (for example, candidiasis); or bacterial, parasitic, and similar microbial infection (for example, disseminated bacteremia; malaria; onchocerciasis; or amebiasis); (10) autoimmune disease (for example, thyroiditis; lupus; Goodpasture's syndrome; allograft rejection; graft versus host disease; or diabetes); and (11) cancer or tumor disease (for example, Hodgkin's disease), but not limited to those.

Treating the inflammatory component of such diseases has been a major goal of the global pharmaceutical industry for a number of decades, and a wide variety of useful treatments have been developed. Examples include the corticosteroids (a range of natural, semisynthetic and synthetic agents designed to mimic the effect of cortisol, including prednisolone, methylprednisolone, dexamethasone, betamethasone, fluticasone and so forth), cyclooxygenase inhibitors (both non-selective or cox-1 selective, such as indomethacin, sulfasalzine and aspirin, and more recently cox-2 selective, such as celecoxib), leukotriene blockers (such as monteleukast) and anti-TNFs (such as modified monoclonal neutralizing antibodies, including infliximab (Remicade™) and adalimumab (Humira™), TNF receptor fusion proteins, such as etanercept (Enbrel™), as well as small molecule TNF-α synthesis inhibitors like thalidomide).

In one embodiment of the present invention, an anti-inflammatory composition comprising a peptide as an active ingredient is provided. The peptide comprises at least one amino acid sequence of SEQ ID NO: 1 to SEQ ID NO: 161, the peptide has above 80% homology with above-mentioned sequences, or the peptide is a fragment of the above-mentioned peptides.

In one embodiment of the present invention, the anti-inflammatory composition may contain 0.1 μg/mg to 1 mg/mg, specifically 1 μg/mg to 0.5 mg/mg, more specifically 10 μg/mg to 0.1 mg/mg of a peptide comprising of amino acid sequence of at least one of SEQ ID NO: 1 to SEQ ID NO: 161, a peptide comprising of amino acid sequence above 80% homology with above-mentioned sequences, or peptide fragment of above-mentioned peptides. When the peptide is contained in the above mentioned range, all the safety and stability of the composition may be satisfied and appropriate in terms of cost-effectiveness.

In one embodiment of the present invention, the composition may have application with all animals including human, dog, chicken, pig, cow, sheep, guinea pig, and monkey.

In one embodiment of the present invention, the medical composition for the use of treatment or prophylaxis of inflammatory disease with an active ingredient that is comprised of a peptide consisting of an amino acid sequence from SEQ ID NO: 1 to SEQ ID NO: 161, a peptide comprising of amino acid sequence above 80% homology with above-mentioned sequences, or peptide fragment of SEQ ID NO: 1, is provided. In one embodiment of the present invention, the pharmaceutical composition may be administered through oral, rectal, transdermal, intravenous, intramuscular, intraperitoneal, in the bone marrow, epidural or subcutaneous means.

Forms of oral administration may be, but not limited to, tablets, pills, soft or hard capsules, granules, powders, solution, or emulsion. Forms of non-oral administration may be, but not limited to, injections, drips, lotions, ointments, gels, creams, suspensions, emulsions, suppository, patch, or spray.

In one embodiment of the present invention, the pharmaceutical composition, if necessary, may contain additives, such as diluents, excipients, lubricants, binders, disintegrants, buffers, dispersants, surfactants, coloring agents, aromatics or sweeteners. In one embodiment of the present invention, the pharmaceutical composition can be manufactured by conventional methods of the industry in the art.

In one embodiment of the present invention, the active ingredient of the medical composition may vary according to the patient's age, sex, weight, pathology and state, administration route, or prescriber's judgment. Dosage based on these factors is determined within levels of those skilled in the art, and the daily dose for example may be, but not limited to, 0.1 μg/kg/day to 1 g/kg/day, specifically 1 μg/kg/day to 10 mg/kg/day, more specifically 10 μg/kg/day to 1 mg/kg/day, more specifically 50 μg/kg/day to 100 μg/kg/day.

In one embodiment of the present invention, the pharmaceutical composition may be administered, but not limited to, 1 to 3 times a day.

In one embodiment of the present invention, a skin external composition for improvement or prevention of skin inflammation is provided. The skin external composition may contain an active ingredient that is a peptide comprising of an amino acid sequence from SEQ ID NO: 1 to SEQ ID NO: 161, a peptide comprising of amino acid sequence above 80% homology with above-mentioned sequences, or peptide fragment of above-mentioned peptides.

In another embodiment of the present invention, a cosmetic composition for improvement or prevention of skin inflammation is provided. The cosmetic composition may contain an active ingredient that is a peptide comprising of an amino acid sequence from SEQ ID NO: 1 to SEQ ID NO: 161, a peptide comprising of amino acid sequence above 80% homology with above-mentioned sequences, or peptide fragment of above-mentioned peptides.

In one embodiment of the present invention, external application composition or cosmetic composition may be provided in all forms appropriate for topical applications. For example, forms can be provided as solutions, emulsions obtained by dispersion of oil phase in water, emulsion obtained by dispersion of water in oil phase, suspension, solid, gel, powder, paste, foam or aerosol. These forms can be manufactured by conventional methods of the industry in the art.

In one embodiment of the present invention, the cosmetic composition may include, within levels that will not harm the main effect, other ingredients that can desirably increase the main effect. In one embodiment of the present invention, the cosmetic composition may additionally include moisturizer, emollient agents, surfactants, UV absorbers, preservatives, fungicides, antioxidants, pH adjusting agent, organic or inorganic pigments, aromatics, cooling agent or antiperspirant. The formulation ratio of the above-mentioned ingredients can be decided by those skilled in the art within levels that will not harm the purpose and the effects of the present invention, and the formulation ratio based on total weight of the cosmetic composition can be 0.01 to 5% by weight, specifically 0.01 to 3% by weight.

In one embodiment of the present invention, a food composition for inflammation prevention or suppression is provided. The food composition may contain with an active ingredient that is a peptide comprising of an amino acid sequence from SEQ ID NO: 1 to SEQ ID NO: 161, a peptide comprising of amino acid sequence above 80% homology with above-mentioned sequences, or peptide fragment of above-mentioned peptides.

In one embodiment of the present invention, food composition is not limited to forms, but for example may be granules, powder, liquid, and solid forms. Each form can be formed with ingredients commonly used in the industry appropriately chosen by those skilled in the art, in addition to the active ingredient, and can increase the effect with other ingredients.

Decision for dosage on the above-mentioned active ingredient is within the level of those skilled in the art, and daily dosage for example may be 1 μg/kg/day to 10 mg/kg/day, more specifically 10 μg/kg/day to 1 mg/kg/day, more specifically 50 μg/kg/day to 100 μg/kg/day, but not limited to these numbers and can vary according to age, health status, complications and other various factors.

In one embodiment of the present invention, a use of prevention or treatment of inflammatory disease with a peptide comprising of an amino acid sequence from SEQ ID NO: 1 to SEQ ID NO: 161, a peptide comprising of amino acid sequence above 80% homology with above-mentioned sequences, or peptide fragment of above-mentioned peptides, is provided.

In one embodiment of the present invention, the method of prevention or treatment of inflammatory disease with applying peptides mentioned above in patients is provided.

In one embodiment of the present invention, a kit for prophylaxis or treatment of inflammatory diseases is provided. The kit may contain: a peptide with anti-inflammatory activity or a composition comprising of the peptide, wherein the peptide comprises any one amino acid sequence of SEQ ID NO: 1 to SEQ ID NO: 161, the peptide has above 80% homology with above-mentioned sequences, or the peptide is a fragment of the above-mentioned peptides; and instructions including at least one of administration dose, administration route, administration frequency, and indication of the peptide or composition.

The terms used herein is intended to be used to describe the embodiments, not to limit the present invention. Terms without numbers in front are not to limit the quantity but to show that there may be more than one thing of the term used. The term “including”, “having”, and “comprising” shall be interpreted openly (i.e. “including but not limited to”).

Mention of range of numbers is used instead of stating separate numbers within the range, so unless it is explicitly stated, each number can be read as separate numbers integrated herein. The end values of all ranges are included in the range and can be combined independently.

Unless otherwise noted or clearly contradicting in context, all methods mentioned herein can be performed in the proper order. The use of any one embodiment and all embodiment, or exemplary language (e.g., that use “like ˜”), unless included in the claims, is used to more clearly describe the present invention, not to limit the scope of the present invention. Any language herein outside of the claims should not be interpreted as a necessity of the present invention. Unless defined otherwise, technical and scientific terms used herein have meaning normally understood by a person skilled in the art that the present invention belongs to.

The preferred embodiments of the present invention are the best mode known to the inventors to perform the present invention. It may become clear to those skilled in the art after reading the statements ahead of the variations in the preferred embodiments. The present inventors hope that those skilled in the art can use the variations adequately and present invention be conducted in other ways than listed herein. Thus, the present invention, as allowed by the patent law, includes equivalents, and variations thereof, of the key points of the invention stated in the appended claims. In addition, all possible variations within any combination of the above-mentioned components are included in the present invention, unless explicitly stated otherwise or contradicting in context. Although the present invention is described and shown by exemplary embodiments, those skilled in the art will understand well that there can be various changes in the form and details without departing from the spirit of the invention and range, defined by the claims below.

Tumor necrosis factor (TNF), particularly TNF-α, is known to be released from inflammatory cells and cause various cytotoxic reactions, immunological reactions and inflammatory reactions. TNF-α is known to be involved in the occurrence and prolongation of many inflammatory and autoimmune diseases and further cause serious septicemia and septic shock when it is released into the blood and acts systemically. Because TNF-α is a factor associated widely with the immune system of a living body, the development of agents inhibiting TNF-α is actively carried out. TNF-α is biosynthesized in an inactive form and becomes an active form by being cleaved by protease; the enzyme responsible for the activation is called a tumor necrosis factor-converting enzyme (TACE). Thus, a substance inhibiting this TACE can treat, improve, or prevent diseases, pathologic conditions, abnormal conditions, troubles, adverse symptoms and the like ascribed to TNF-α. High-mobility group box 1 (HMGB1) protein exists in high concentrations in thymus, lymph nodes, testes, and in fetal liver, and with exception to liver and brain cells, usually exists inside of the nucleus. The said HMGB1 protein has 3 domains consisting of A-box, B-box, and C-terminal.

It was reported by Tracey et al., 1999 that HMGB1 protein has a role as a cytokine which induces inflammation, and the mechanism of said HMGB1's inflammation induction is by an external stimulus causing acetylation of HMGB1 which then moves from the nucleus into the cytoplasm. Afterward, it is known to be secreted out of the cell, or secreted out from the cell in necrosis. (Bonaldi T et al., EMBO J, (22)5551-60, 2003). The invention is further described by the figures, the following examples and experiments, which are solely for the purpose of illustrating specific embodiments of this invention, and are not to be construed as limiting the scope of the invention in any way.

Proteins, Nucleic acids, Peptides or virus, etc. have big potentials to be used as therapeutic substances. However, their uses are limited because they cannot penetrate tissues and cell membrane due to molecular level sizes. Although, the size of molecules is small, they cannot penetrate lipid-bilayer due to structure or characteristics of the molecules. Thus, through the use of electroporation, heat shock, etc., there were attempts to transport proteins, nucleic acids, peptides or viruses inside the cell; it was difficult to transfer those without neither damaging cell membrane nor keeping the active states of above molecules. There have been many studies conducted since TAT (Trans-Activating Transcriptional activator) protein derived from HIV (Human Immuno-deficiency Virus) has shown to work as cell penetrating peptide which can transport huge active substances inside the cell. Specifically, there have been studies conducted about substances that can transport huge molecules such as proteins, nucleic acids, peptides or virus inside the cell without causing any toxicity, unlike TAT protein which causes toxicity inside the cell. Therefore, the present invention was completed as present inventors have found that peptides derived from telomerase have outstanding efficacy as cell penetrating peptide without a noticeable toxicity.

In one embodiment of the present invention, cell penetrating peptide comprising any one amino acid sequences of SEQ ID NO:71, SEQ ID NO:82, SEQ ID NO:96, SEQ ID NO:142, SEQ ID NO:153, and SEQ ID NO:164, a peptide having above 80% homology of amino acid sequence with above-mentioned sequences, or a fragment of the above-mentioned peptides, is provided.

In one embodiment of the present invention, a pharmaceutical composition comprising peptide as a drug delivery system to transport more than one active ingredient is provided, wherein the peptide comprises any one amino acid sequence of SEQ ID NO:71, SEQ ID NO:82, SEQ ID NO:96, SEQ ID NO:142, SEQ ID NO:153, and SEQ ID NO:164, the peptide has above 80% homology with above-mentioned sequence, or the peptide is a fragment of above-mentioned peptide.

A peptide comprising any one amino acid sequence of SEQ ID NO:71, SEQ ID NO:82, SEQ ID NO:96, SEQ ID NO:142, SEQ ID NO:153, and SEQ ID NO:164, a fragment of above-mentioned peptide, or a peptide having above 80% homology with above-mentioned sequence, is safe and has outstanding efficacy as cell penetrating peptide.

Therefore, the peptide can be conjugated with a drug to transport the drug inside the cell.

In one embodiment of the present invention, a conjugate of a peptide and an active ingredient to be transported is provided, wherein the peptide comprises any one amino acid sequence of SEQ ID NO:71, SEQ ID NO:82, SEQ ID NO:96, SEQ ID NO:142, SEQ ID NO:153, and SEQ ID NO:164, the peptide is a fragment of above-mentioned peptide, or the peptide has above 80% homology with above-mentioned peptide. In one embodiment of the present invention, an active ingredient may be at least one selected from proteins, nucleic acids, peptides, lipids, glycolipids, minerals, sugars, contrast substances, drugs and chemical compounds. In one embodiment of the present invention, the active ingredients may be peptides. In one embodiment of the present invention, the active ingredients may be cytokines, antibody, antibody fragments, therapeutic enzymes, soluble receptors, or ligands.

A cell penetrating peptide disclosed herein means a peptide which can transport cargo from in vitro and/or in vivo to inside the cell. A “cargo” disclosed herein comprises all the substances that can be transported inside the cell via conjugation with a cell penetrating peptide, For example, all the substances which want to increase cell penetrating efficacy, specifically drugs, cosmetics, or active ingredients of health food, more specifically substances which cannot be transported inside the cell via general route, more specifically, sugars, nano-particles, biological formulation, viruses, contrast substances or other chemical compounds which can have proteins, nucleic acids, peptide, minerals, glucose as an example, but not limited to those. A “drug” disclosed herein is a broad concept including a substance to be transported for alleviation, prophylaxis, treatment or diagnosis of diseases, wounds, or specific symptom.

A “carrier peptide” disclosed herein is a peptide which can transport active ingredients to a targeted site via conjugation with active ingredients. In one embodiment of the present invention, protein or peptide as a cargo comprises one or more of hormone, hormone analogue, enzyme, enzyme inhibitors, signal transfer proteins (or peptides), antibody and vaccine, but not limited to those. In one embodiment of the present invention, a nucleic acid is a molecule that can be spontaneous or artificial DNA or RNA molecules, either single-stranded or double-stranded. The nucleic acid molecule can be one or more nucleic acids of same type (for example, having a same nucleotide sequence) or nucleic acids of different types. The nucleic acid molecules comprise one or more DNA, cDNA, decoy DNA, RNA, siRNA, miRNA shRNA, stRNA, snoRNA, snRNA PNA, antisense oligomer, plasmid and other modified nucleic acids, but not limited to those. In one embodiment of the present invention, virus comprises the whole virus or the core of virus which includes nucleic acids of the virus. In one embodiment of the present invention, a chemical substance is a broad indication comprising a natural or synthetic substance which can act as a drug.

A phenomenon where specific DNA expression is controlled by double stranded RNA (dsRNA) in a process of DNA expression is called RNA interference; RNAi. Since the phenomenon was first discovered in C. elegans in 1998, it was found that the phenomenon is common in plants, fruit flies and mammals (Fire et al., Nature, 391:806-811, 1998; Novina & Sharp, Nature, 430:161-164, 2004).

RNA interference is mediated by dsRNA having 19-25 bps that enters the cells, upon which it combines with RISC (RNA-induced silencing complex). The binding of the anti-sense strand of dsRNA to the complementary mRNA sequence triggers degradation of the target mRNA by the endonuclease enzyme found in RISC complex. (Rana, T. M., Nat. Rev. Mol. Cell Bio., 8:23-36, 2007; Tomari, Y. and Zamore, P. D., Genes Dev., 19: 517-529, 2005). In other words, siRNA is involved in RNA interference by suppressing production of specific protein and thereby interfering with DNA expression. siRNA consisting of 19˜23 nucleotides, forms a base pair according to complementary order of mRNA for specific messenger RNA (mRNA) to form double-stranded RNA. After then, the double-stranded RNA is specially disintegrated at the same time messenger RNA is removed from the cells. siRNA has been spotlighted as a substance for gene therapy because it showed an outstanding effect in suppressing expression of specific DNA in recent animal studies. siRNA with higher activation and precise selection of DNA, has been studied for last 20 years and it is expected to replace the antisense oligonucleotides which are currently being used as a remedy. Therefore, many pharmaceutical companies are now developing a siRNA based remedy. Compared to the existing anti-sense oligonucleotides, siRNA is known to inhibit gene expression with 10 times less amount and inhibit target genes only with an outstanding selectivity of genes. siRNA technique, especially for the treatment purpose, has a great advantage as it can be easily designed compared to other drugs and has characteristics such as high target selectivity and inhibition of specific gene expression. Also, since suppression of gene expression by RNA interference utilizes the mechanism naturally present in vivo, toxicity is low. However, siRNA has a disadvantage that it cannot be easily transported into a cell as it cannot penetrate the cell membrane as it is anionic and easily broken down in a short period of time due to low stability in vivo. This disadvantage of siRNA can be solved by conjugating with a carrier peptide disclosed herein.

In one embodiment of the present invention, drugs transported inside the cell by cell penetrating peptide can comprise one or more drug transporters such as liposome, micelle, nano-particles, magnetic-particles or Quantum Dot.

The term “contrast substance” disclosed herein is a broad indication comprising all the substances used to contrast structures or fluids within the body in medical imaging. An appropriate contrast substance comprises radiopaque contrast agent, paramagnetic contrast agent, superparamagnetic contrast agent, CT (computed tomography) and other contrast substances, but not limited to those. For example, a radiopaque contrast agent (for x-ray imaging) will comprise inorganic iodine compound and organic iodine compound (for example, diatrizoate), radiopaque metals and their salts (for example, silver, gold, platinum, etc.) and other radiopaque compounds (for example, calcium salts, barium salt such as barium sulfate, tantalum and oxidized tantalum). An appropriate paramagnetic contrast substance (for MR imaging) comprises gadolinium diethylene triaminepentaacetic acid (Gd-DTPA) and its derivatives, other gadolinium, manganese, iron, dysprosium, copper, europium, erbium, chrome, nickel and cobalt complex, for example, 1,4,7,10-tetraazacyclododecan-N,N′,N″,N′″ tetraacetic acid (DOTA), ethylenediaminetetraacetic acid (EDTA), 1,4,7,10-tetraazacyclododecan-N,—N′, N″-triacetic acid (D03A),1,4,7-triazacyclononane-N,N′, N″-TRIACETIC ACID (NOTA), 1,4,8,10-tetraazacyclotetradecane-N,N′,N″,N″′-tetraacetic acid (TETA), hydroxybenzylethylene-diamine diacetic acid (HBED). An appropriate superparamagnetic contrast substance (for MR imaging) comprises magnetite, superparamagnetic iron oxide (SPIO), ultrasmall superparamagnetic iron oxide (USPIO) and monocrystalline iron oxide. Other appropriate contrast substances are iodinated, non-iodinated, ionic and non-ionic CT contrast agents, a contrast substance like spin-label or diagnostically effective agent.

Other examples of contrast substances comprise β-galactosidase, Green Fluorescent Protein, Cyan Fluorescent Protein, luciferase, but not limited to those, and a marker gene which codes for protein which can be easily detected when expressed within cells. Various labels such as radionuclide, flour, enzyme, enzyme-substrate, enzyme cofactor, enzyme inhibitor, ligands (especially hapten) can be used.

In one example of the present invention, a contrast substance IS ferrocenecarboxylic acid of the below chemical formula 2. The structure of ferrocene is shown in the chemical formula 1.

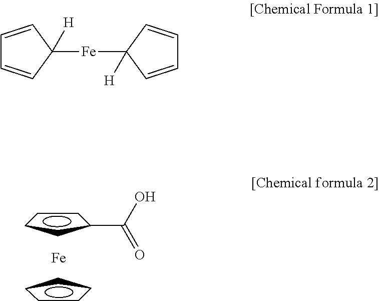

In one example of the present invention, a conjugate of cell penetrating peptide and a contrast substance is Ferrocenecarboxylic-pep (Ferrocenecarboxylic-pep) shown in the below chemical formula 3.

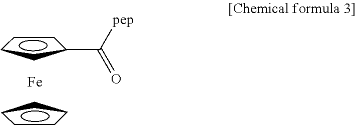

In one embodiment of the present invention, a peptide or composition can be fused with one or more detectable labels. Labels may be compounds which can be detected in chemical, physical or enzymatic responses, or compounds which generate signals directly or indirectly in the responses. Labeling and detecting after then can be performed according to the known method in the art (For example, Sambrook, J., and Russel, D. W. (2001); and Lottspeich, F., and Zorbas H. (1998) Bioanalytik, Spektrurn Akademischer Verlag, Heidelberg/Berlin, Germany). Labels comprise fluorescent label, enzyme label, chromogenic label, luminescence label, radiation label, hapten, biotin, metal complex, metal and colloidal gold, but not limited to those. All forms of these labels are well known in this field of work, they can be commercially obtained from various suppliers.

In one embodiment of the present invention, a cargo can be directly combined with the peptide. In another embodiment of the present invention, a cargo can be combined to the peptide via various types of bonds such as covalent or non-covalent bonds. A cargo, for example, can be combined to the N-terminal or C-terminal of the peptide in one embodiment of the present invention. For example, a cargo can be bonded to the peptide by disulfide bonds or covalent bonds. The covalent bonds are the bonds that a cargo can be bonded to a-amine of N-terminal glutamate, or amine of C-terminal Lysine residues. Also, a peptide and a cargo can be combined via a non-covalent bond, which can have either a peptide or a cargo can encapsulate the other as a capsule form.

In another embodiment of the present invention, a peptide can be combined with a cargo via a linker. For example, a peptide can be combined with a cargo by binding a cargo to a linker after introducing a linker such as Hynic(6-hydrazinopyridine-3-carboxylic acid) linker, to the a-amine of N-terminal glutamate, or amine of C-terminal Lysine residues.

In another embodiment of the present invention, when a cargo is DNA or RNA, SH group (thiol group) is introduced to the peptide, and maleimide group is introduced to DNA or RNA, then, SH group of the peptide and maleimide group of DNA or RNA are combined, thus creating a bond between the cargo and the peptide.

In another embodiment of the present invention, when a cargo is a peptide or protein, DNA which expresses a cargo is combined with DNA which expresses a carrier peptide, and by expressing this, a cargo and a peptide can be combined as a form of fusion protein. Specific examples of combination by a fusion protein are as follows: when manufacturing primer for production of fusion protein, a nucleotide coding a carrier peptide is attached in front of a nucleotide expressing a cargo, and the obtained nucleotide is inserted to a vector such as pET vector using a restriction enzyme, and the nucleotide is expressed by transformation into a cell such as BL-21(DE3). At this time, a fusion protein is to be effectively expressed by treating it with an expression inducing agent like IPTG (isopropyl-1-thio-P-D-galactopyranoside). Then, the expressed fusion protein is purified by His tag purification, and is dialyzed with PBS, and is added to a kit to be concentrated by centrifugation under such condition for 5 to 20 mins. at 2,000 to 4,000 rpm.

In one embodiment of the present invention, a carrier peptide is combined with dying substances, fluorescent substances, specifically FITC (fluorescein isothiocyanate) or GFP (Green Fluorescent Protein). In one embodiment of the present invention, FITC is combined with amino group (NH3+) of lysine at N-terminal or C-terminal of a carrier peptide. In the case of a peptide, where lysine does not exist at its terminal, the peptide can be combined with FITC via a linker including Lysine.

The carrier peptide disclosed herein which is the peptide comprising any one amino acid sequence of SEQ ID NO:71, SEQ ID NO:82, SEQ ID NO:96, SEQ ID NO:142, SEQ ID NO:153, and SEQ ID NO:164, or the peptide having above 80% homology of amino acid sequence with above-mentioned peptides, or a fragment of above-mentioned peptide, can be combined with a cargo at a mole fraction of 1:1, but it can be combined at mole fraction other than 1:1. For example, a mole fraction of CPP and a cargo may be more than 2:1, specifically, more than 2:1, more than 3:1, more than 4:1, more than 5:1, more than 6:1, more than 7:1, more than 8:1, more than 9:1 or more than 10:1. This means that numerous carrier peptide molecules can be combined with a cargo molecule. The numerous carrier peptide molecules can be combined in series or in parallel. “Combined in series” means that a carrier peptide and a cargo molecule are to be combined at terminal amino acids. “Combined in parallel” means that they are to be combined at a site other than terminal amino acids. On the other hand, the mole fraction of a carrier peptide and a cargo may be more than 1:2. This means that a carrier peptide molecule can be combined with numerous number of a cargo molecule. For example, a mole fraction of a carrier peptide and a cargo may be 1:2, specifically, more than 1:2, more than 1:3, more than 1:4, more than 1:5, more than 1:6, more than 1:7, more than 1:8, more than 1:9 or more than 1:10.

A movement pathway of the peptide combined with Fluorescein isothiocyanate can be easily found. Therefore, a carrier peptide in one embodiment of the present invention is to be used for cell imaging or detecting a pathway of drug delivery inside a cell.

In one embodiment of the present invention, a use of the peptide as a drug delivery carrier to transport more than one active ingredient is provided, wherein the peptide comprises any one amino acid sequence of SEQ ID NO:71, SEQ ID NO:82, SEQ ID NO:96, SEQ ID NO:142, SEQ ID NO:153, and SEQ ID NO:164, or the peptide is a fragment of above-mentioned peptide, or the peptide has above 80% homology of amino acid sequence with above-mentioned peptide. The use may indicate therapeutic or non-therapeutic use.

In one embodiment of the present invention, a method of delivering drugs inside a cell of a subject comprising a step of administering a composition comprising a drug; and the peptide is provided; wherein the peptide comprises any one amino acid sequence of SEQ ID NO:71, SEQ ID NO:82, SEQ ID NO:96, SEQ ID NO:142, SEQ ID NO:153, and SEQ ID NO:164, or the peptide is a fragment of above-mentioned peptide, or the peptide has above 80% homology of amino acid sequence with above-mentioned peptide.

In one embodiment of the present invention, a method of detecting drug delivery pathway comprising a step of applying the peptide and a contrast substance to a subject is provided; wherein the peptide comprises any one amino acid sequence of SEQ ID NO:71, SEQ ID NO:82, SEQ ID NO:96, SEQ ID NO:142, SEQ ID NO:153, and SEQ ID NO:164, or the peptide is a fragment of above-mentioned peptide, or the peptide has above 80% homology of amino acid sequence with above-mentioned peptide.

In one embodiment of the present invention, a method of detecting drug delivery pathway comprising a step of applying of the conjugate of the peptide and a contrast substance to a subject is provided; wherein the peptide comprises any one amino acid sequence of SEQ ID NO:71, SEQ ID NO:82, SEQ ID NO:96, SEQ ID NO:142, SEQ ID NO:153, and SEQ ID NO:164, or the peptide is a fragment of above-mentioned peptide, or the peptide has above 80% homology of amino acid sequence with above-mentioned peptide.

In one embodiment of the present invention, a kit for drug delivery into a cell of a subject containing the composition and an instruction is provided, wherein the composition comprises a conjugate of a peptide of the invention and a drug for delivery, wherein the peptide comprises any one amino acid sequence of SEQ ID NO:71, SEQ ID NO:82, SEQ ID NO:96, SEQ ID NO:142, SEQ ID NO:153, and SEQ ID NO:164, or the peptide is a fragment of above-mentioned peptide, or the peptide has above 80% homology of amino acid sequence with above-mentioned peptide, wherein the instruction includes at least one of administration dose, administration route, administration frequency, and indication of the composition.

In one embodiment of the present invention, cosmetic or food composition comprising an active ingredient; and the peptide is provided; wherein the peptide comprises amino acid sequence of SEQ ID NO:71, SEQ ID NO:82, SEQ ID NO:96, SEQ ID NO:142, SEQ ID NO:153, and SEQ ID NO:164, the peptide has above 80% homology of amino acid sequence with above-mentioned sequence, or the peptide is a fragment of the above-mentioned peptides. In another embodiment of the present invention, cosmetic or food composition comprising a conjugate of the peptide and active ingredients is provided; wherein the peptide comprises any one amino acid sequence of SEQ ID NO:71, SEQ ID NO:82, SEQ ID NO:96, SEQ ID NO:142, SEQ ID NO:153, and SEQ ID NO:164 the peptide has above 80% homology of amino acid sequence with above-mentioned sequence, or the peptide is a fragment of the above-mentioned peptides.

In one embodiment of the present invention, pharmaceutical, cosmetic or food composition with an outstanding ability to transport active ingredients inside a cell, comprising a conjugate of the peptide and an active ingredient, is provided; wherein the peptide comprises any one amino acid sequence of SEQ ID NO:71, SEQ ID NO:82, SEQ ID NO:96, SEQ ID NO:142, SEQ ID NO:153, and SEQ ID NO:164, the peptide has above 80% homology of amino acid sequence with above-mentioned sequence, or the peptide is a fragment of the above-mentioned peptides.

Mitochondria, as a central organelle in energy metabolism of a eukaryotic cell, is a first known intracellular organelle to be related to human diseases (Luft R, Ikkos D, Palmieri G, Ernster L, Afzelius B: A case of severe hypermetabolism of non-thyroid origin with a defect in the maintenance of mitochondrial respiratory control: a correlated clinical, biochemical, and morphological study, J Clin Invest 41:1776-804, 1962).

Since the mitochondria play an important role in control of energy metabolism of cell and apoptosis, they act as a major target for various therapeutic drugs. Also, this organelle is involved in control of the calcium concentration inside the cell, the mitochondrial respiratory chain acts as an electron transport system which is important in energy production, and it causes production of reactive oxygen species. As a result, the abnormal mitochondrial function has a close relationship with adult diseases such as diabetes, cardiomyopathy, infertility, blindness, renal/liver diseases, and stroke (Modica-Napolitano K S, Singh K K: April mitochondria as targets for detection and treatment of cancer. Expert Rev Mol Med 11:1-19, 2002). Also, it is being suggested that Mitochondrial genetic mutations to be involved in the outbreak of aging, degenerative neuronal disease and cancer etc.

The mitochondria targeting delivery system can be provided according to the one embodiment of the present invention may comprise any one of conjugates mentioned above, wherein the carrier peptide moves into intracellular mitochondria locally and performs a role of local intracellular mitochondria delivering the mentioned active ingredients, wherein the peptide having above 80% homology of amino acid sequence with above-mentioned sequence and the fragment of the same are the peptides that maintain mitochondria targeting delivery system, the above-mentioned mitochondria targeting peptide may be the peptide having any one amino acid sequences of SEQ ID NO:71, SEQ ID NO:82, SEQ ID NO:96, SEQ ID NO:142, SEQ ID NO:153, and SEQ ID NO:164.

The mitochondria activity adjusting composition can be provided, wherein the composition comprises a conjugate of a peptide of the invention and a carrier peptide for delivery, wherein the carrier peptide moves into intracellular mitochondria locally and performs a role of local intracellular mitochondria delivering the mentioned active ingredients, wherein the peptide having above 80% homology of amino acid sequence with above-mentioned sequence and the fragment of the same are the peptides that maintain mitochondria targeting delivery system, the above-mentioned mitochondria targeting peptide may be the composition having any one amino acid sequences of SEQ ID NO:71, SEQ ID NO:82, SEQ ID NO:96, SEQ ID NO:142, SEQ ID NO:153, and SEQ ID NO:164.

The mitochondria activity adjusting composition according to the one embodiment of the present invention, wherein the composition for the treatment of a mitochondrial related disease or disorder, prevention, inhibitory of progress, or relief of symptoms as an a pharmaceutical composition, wherein the active ingredient will be treated for a mitochondrial related diseases or disorder, prevention, inhibitory of progress, or relief of symptoms.

The “Mitochondrial related diseases” disclosed herein comprise Huntington's disease, amyotriophic lateral sclerosis, MELAS (Mitochondrial Encephalomyopathy with Lactic Acidemia and Stroke-like episodes); MERRF (Myoclonus, epilepsy, and myopathy with ragged red fibers; NARP/MILS (Neurogenic muscular weakness, ataxia, retinitis pigmentosa/Maternally-inherited leigh syndrome); LHON (Lebers hereditary optic neuropathy); KSS (Kearns-Sayre Syndrome); PMPS (Pearson Marrow-Pancreas Syndrome); CPEO (Chronic progressive external opthalnoplegia); Reye's syndrome; Alper's syndrome; Multiple mtDNA deletion syndrome; mtDNA depletion syndrome; Complex I deficiency; Complex II (SDH) deficiency; Complex Ill deficiency; Cytochrome c oxidase (COX, Complex IV) deficiency; Complex V deficiency; Adenine nucleotide translocator (ANT) deficiency; Pyruvate dehydrogenase (PDH) deficiency; Ethyl malonic acid aciduria having lactic acid acidemia; 3-methyl glutaconic acid aciduria having lactic acid acidemia; refractoriness epilepsy representing a decline during infection; Asperger's syndrome representing a decline during infection; Autism representing a decline during infection; Attention deficit hyperactivity disorder (ADHD); Cerebral palsy representing a decline during infection; Alexia representing a decline during infection; Maternal hereditary thrornbocytopenia; Leukemia; MNG-IE (Mitochondrial myopathy, peripheral and autonomic neuropathy, gastrointestinal dysfunction, and epilepsy); MARIAJ:IS syndrome (mitochondrial ataxia, recrudescent infection, aphasia, hypouricemial lypomyelination, seizure and dicarboxylic acid aciduria); ND6 dystonia; Cyclic vomiting syndrome representing a decline during infection; 3-hydroxyisobutyric acid aciduria having lactic add ac1demmDiabetes having lactic acid acidemia; Uridine reactive neural syndrome (URNS); Familial bilateral striatum necrosis (fBSN); Hearing loss related with aminoglycoside; Relaxed myocardiopathy; Spleen lymphoma; Wolframs syndrome; Multiple mitochondria DNA deletions syndrome; and Renal tubular acidosis/diabetes/ataxia syndrome, but not limited to those.

In another embodiment of the present invention, nucleic acid molecules encoding above-mentioned polypeptides are provided. The nucleic acid molecules, for example, have base sequences of GAA GCG CGC CCG GCG CTG CTG ACC AGC CGC CTG CGC TIT ATT CCC AAA (Sequence number 181). The nucleic acids can be introduced into the host cell according to a known method to those skilled in the art. For example, the known methods may be transformation method by calcium phosphate method, liposome, electroporation, contacting a virus and a cell, or micro injection directly into the cell, etc. The host cell is higher eukaryotic cell, for example, mammalian cells, or lower eukaryotic cells, such as a yeast cell, or prokaryotic cells, such as a bacterial cell. The prokaryotic host cells appropriate for transformation may be the species which belong to E. coli, Bacillus subtillis, Salmonella typhimurium, Pseudomonas, Streptomyces, and Micro bacteria species, as examples.

The vector including above-mentioned nucleic acid molecules 1 s generally recombinant expression vector and it comprises, origin of replication enabling a host cell transformation, and a selectable marker (for example, dihydrofolate reductase for eukaryotic cell culture, or tolerance of neomycin, tolerance of tetra-cycline or ampicillin in E. coli, or S. cerevisiae TRPJ gene), and the promoter for controlling transcription of protein coating sequences. Available expression vectors are, for example, known bacterial plasmids such as SV40, derivatives of pcDNA, and known bacterial plasmids such as colE1, pCR1, pBR322, pMal-C2, pET, pGEX (Smith, et al., Gene 67:31-40 (1988)), plasmids such as prvfB9 and its derivative RP4phage DNA which is the same as numerous derivatives of phage I such as NM989, phage DNA such as M13 and single-stranded phage DNA of filament type; yeast plasmid, for example, phage DNA or vector induced from a combination of modified plasmid for using expression suppression sequences and phage DNA. The mammalian expression vectors comprise origin of replication, an appropriate promoter and an enhancer. Also, they can comprise compulsory ribosome binding sites, polyadenylation sites, splice donor, and receptor part, transcription termination sequences, and 5′ planking non-transcriptional sequences, The mammalian expression vectors can comprise an inducible promoter, for example, a vector containing dihydrofolate reductase promoter, any expression vectors containing DHFR expression cassette or DHFR/methotrexate co-amplification vector such as pED. (Randal J, Kaufman, 1991, Randal J. Kaufman, Current Protocols in Molecular Biology, 16, 12(1991)), Or, glutamine synthetase/methionine sulfoximine co-amplification vector, for example, pEE14 (Celltech), Epstein-Barr-Virus (EBV), or a vector directing episomal expression under the control of nuclear antigen (EBNA), for example, pREP4 (Invitrogen), pCEP4 (Invitrogen), plviEP4 (Invitrogen), pREP8(Invitrogen), pREP9(Invitrogen) and pEBVHis (Invitrogen) can be used. Selectable mammalian expression vectors are Re/CMV (Invitrogen) and pRc/RSV (Invitrogen) etc. Vaccinia vims mammalian expression vectors which can be used in the present invention are pSC11, pMJ601, pTKgptF1S, etc.

Yeast expression vector system to be used in the present invention is non-fusion pYES2 vector (Invitrogen), fusion pYESHisA, B, C (Invitrogen), pRS vector, etc.

The above-mentioned vectors can be introduced to various cells, such as mammalian cells which is especially the human derived cells, or bacteria, yeast, fungi, insects, nematodes, and plant cells. The examples of appropriate cells are VERO cell, HELA cell, for example, ATCC No. CCL2, CHO cell line, for example, ATCC No. CCL61, COS cell, for example COS-7 cell and ATCC No. CRL 1650 cell, VL38, BHK, HepG-2, 3T3, for example, ATCC No. CRL6361, A549, PC12, K562 cell, 293 cell, Sf9 cell, for example, ATCC No. CRL1711 and Cv1 cell, such as ATCC No. CCL70, etc.

Other appropriate cells to be used in the present invention are prokaryotic host cell strain, for example, the strains belonging to E. coli (e.g. DH5-α strain), Bacillus subtilis, Salmonella typhimurium, Pseudomonas, Streptomyces, and Staphylococcus.

In one embodiment of the present invention, the composition may contain 0.1 μg/mg to 1 mg/mg, specifically 0.1 μg/mg to 0.5 mg/mg, more specifically 10 μg/mg to 0.1 mg/mg of a peptide comprising any one amino acid sequence of SEQ ID NO:71, SEQ ID NO:82, SEQ ID NO:96, SEQ ID NO:142, SEQ ID NO:153, and SEQ ID NO:164, a peptide comprising amino acid sequence above 80% homology with above-mentioned sequence, or a fragment of above-mentioned peptide. When the peptide is contained in the above-mentioned range, all the safety and stability of the composition can be satisfied and appropriate in terms of cost-effectiveness.

In one embodiment of the present invention, the composition may have application with all animals including human, dog, chicken, pig, cow, sheep, guinea pig, and monkey.

In one embodiment of the present invention, the pharmaceutical composition may be administered through oral, rectal, transdermal, intravenous, intramuscular, intraperitoneal, in the bone marrow, epidural or subcutaneous means.

Forms of oral administration may be, but not limited to, tablets, pills, soft or hard capsules, granules, powders, solution, or emulsion. Forms of non-oral administration can be, but not limited to, injections, drips, lotions, ointments, gels, creams, suspensions, emulsions, suppository, patch, or spray.

In one embodiment of the present invention, the pharmaceutical composition, if necessary, may contain additives, such as diluents, excipients, lubricants, binders, disintegrants, buffers, dispersants, surfactants, coloring agents, aromatics or sweeteners. In one embodiment of the present invention, the pharmaceutical composition may be manufactured by conventional methods of the industry in the art.

In one embodiment of the present invention, the active ingredient of the medical composition may vary according to the patient's age, sex, weight, pathology and state, administration route, or prescriber's judgment. Dosage based on these factors is determined within levels of those skilled in the art, and the daily dose for example may be, but not limited to, 0.1 μg/kg/day to 1 g/kg/day, specifically, 1 μg/kg/day to 0.1 μg/kg/day, more specifically 50 μg/kg/day to 1000 μg/kg/day. In one embodiment of the present invention, the pharmaceutical composition may be administered, but not limited to, 1 to 3 times a day.

In one embodiment of the present invention, cosmetic composition may be provided in all forms appropriate for topical applications. For example, forms may be provided as solutions, emulsions obtained by dispersion of oil phase in water, emulsion obtained by dispersion of water in oil phase, suspension, solid, gel, powder, paste, foam or aerosol. These forms may be manufactured by conventional methods of the industry in the art.

### EXAMPLES

### Example 1

### Synthesis of PEP-1 and Measurement of Anti-Inflammatory Activities of PEP-1 (SEQ ID NO: 1)

### Experiment 1. Synthesis of PEP-1 CSEO ID NO: 1)

A peptide comprised of 16 amino acids with the chemical formula IV as below having the sequence SEQ ID: 1 (PEP-1) derived from human telomerase was synthesized:

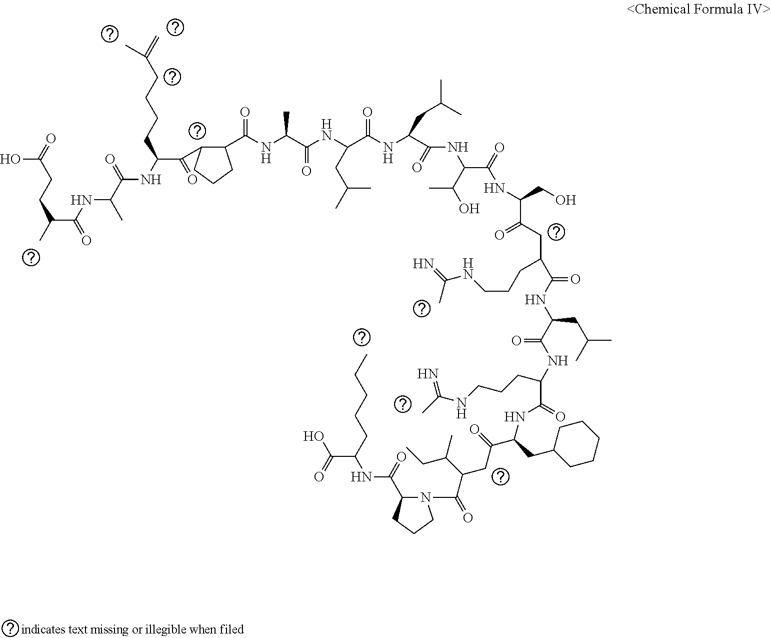

SEQ ID NO: 1 (PEP-1) was synthesized according to the existing method of solid phase peptide synthesis. In detail, the peptides were synthesized by coupling each amino acid from C-terminus through Fmoc solid phase peptide synthesis, SPPS, using ASP48S (Peptron, Inc., Daej eon ROK). Those peptides with their first amino acid at the C-terminus being attached to resin were used as follows:

NH2-Lys(Boc)-2-chloro-Trityl Resin

NH2-Aia-2-chloro-Trityl Resin

NH2-Arg(Pbf)-2-chloro-Trityl Resin

All the amino acid materials to synthesize the peptide were protected by Fmoc at the N-terminus, and the amino acid residues were protected by Trt, Boc, t-Bu (t-butylester), Pbf (2,2,4,6,7-pentamethyl dihydro-benzofuran-5-sulfonyl) that can be dissolved in acid. Such as:

Fmoc-Aia-OH, Fmoc-Arg(Pbf)-OH, Fmoc-Giu(OtBu)-OH, Fmoc-Pro-OH, Fmoc-Leu-OH, FmocIIe-OH, Fmoc-Phe-OH, Fmoc-Ser(tBu)-OH, Fmoc-Thr(tBu)-OH, Fmoc-Lys(Boc)-OH, Fmoc-15 Gln(Trt)-OH, Fmoc-Trp(Boc)-OH, Fmoc-Met-OH, Fmoc-Asn(Trt)-OH, Fmoc-Tyr(tBu)-OH, Fmoc-Ahx-OH, Trt-Mercaptoacetic acid.

HBTU[2-(1H-Benzotriazole-1-yl)-1,1,3,3-tetamethylaminium hexafluorophosphate]I HOBt [N-Hydroxybenzotriazole]/NMM [4-Methylmorpholine] were used as the coupling reagents. In 20% of DMF, piperidine was used to remove Fmoc. In order to remove the protection from residue or to separate the synthesized peptide from Resin, cleavage cocktail [TFA (trifluoroacetic acid)/TIS (triisopropylsilane)/EDT (ethanedithiol)/H20=92.5/2.5/2.5/2.5] was used.

Synthesized the peptide by using the solid phase scaffold combined to starting amino acid with the amino acid protection, reacting the corresponding amino acids separately, washing with solvent and deprotected, and repeating the process. After cutting off the synthesized peptide from the resin, it was purified by HPLC and verify for synthesis by MS, and then freeze-dried.

Specific synthesis process of PEP 1 is described by the following.

1) Coupling

Melted the amino acid (8 equivalent) protected with NH2-Lys(Boc)-2-chloro-Trityl Resin, and coupling agent HBTU (8 equiv.)/HOBt (8 equiv.)/NMM (16 equiv.) and added to DMF, then let react in room temperature for 2 hours, then washed with DMF, MeOH, and DMF in that order.

2) Fmoc deprotection

Added 20% piperidine in DMF and reacted in room temperature for 5 minutes 2 times, then washed with DMF, MeOH, and DMF in that order.

3) Make the basic framework of peptide by repeating reactions 1 and 2 repeatedly.

4) Cleavage: Add Cleavage Cocktail to the completely synthesized peptide and separated the peptide from the resin.

5) Add cooling diethyl ether into obtained mixture, and then centrifugation is used to precipitate gathered peptide.

6) After purification by Prep-HPLC, check the molecular weight by LC/MS and lyophilize to produce in powder form.

### Experiment 2: Anti-Inflammatory Activity Measurement of PEP 1

Cell Lines Culture

Raw 264.7 macrophage cell (KCBL, 40071) from Korea Cell Bank was maintained in Dulbecco's modified Eagle's medium (DMEM; PAA, Austria) containing 10% fetal bovine serum (FBS; Gibco Laboratories), 100 unit/mL of streptomycin, and penicillin (Gibco Laboratories) at 37° C. with 5% C02•Raw264. 7 cells were seeded into a 96-well plate at a density of 1×106 cells/mL and incubated overnight. On the following day, the medium was replaced with fresh medium and 51-Jg/mL of peptide (obtained as described in Experiment example 1) was added to the cells. After 30 min incubation of cells with the peptide 50 1-JL of LPS (to a final concentration of 1 1-Jg/mL) was added, and cells were incubated for additional 24 hr. The experimental sample with the induction of inflammatory response was treated with 1 1-Jg/mL mL lipopolysaccharide (LPS; Sigma, USA) and control sample was treated with phosphate buffered saline (PBS; pH 7.2). The supernatant samples from each condition was collected in eppendorf tubes and subjected to further analysis.

### Experiment 2-1. NO Level Analysis

The level of nitric oxide (NO) was measured in Raw 264.7 cell (1×10⁶ cell/ml) using Griess reagent system (Promega, USA). Culture medium of 50 μl was added to a 96-well plate and Griess reagent I (NED) solution and Griess reagent II (Sulfaniliamide solution) are added in the same amount. After 10 min incubation of cells with the reagents, the optical density at 540 nm was measured within 30 min using a microplate reader (Molecular Devices, USA). The concentration of NO was calculated by using a standard curve (0 . . . , 100 μM) of sodium nitrite.

As shown in Table 3 below, stimulation of cells with LPS increased the expression of NO, but in co-treatment with LPS and Pep1, the expression level of NO mentioned above decreased. NO is produced during inflammation, and the result showing Pep1 reduced NO level to 65% of the control strongly support the anti-inflammatory effect of Pep1.

TABLE 3
The measurement of anti-inflammatory effect
of human telomerase derived PEP 1

| | | NO Expression | Decreased NO |
|---|---|---|---|
| | | Level of | Expression |
| Test sample | | control (%) | Level (%) |
| PBS | | 0 | — |
| LPS 1 μg/mL | PBS | 100 | 0 |
| | PEP 1 (0.5 μg/mL) | 35 | 65 |

### Experiment 2-2. Analysis of Cytokine Inhibitory Effect

To investigate the effect of PEP1 on inhibiting pro-inflammatory cytokine production RAW 264.7 cell were pre-treated with PEP 1 at a concentration of 5 μg/mL challenged with LPS at a concentration of 1 μg/mL, and cells were further incubated for 24 hr. The supernatant samples containing cell culture medium was collected and analyzed for the cytokine levels using ELISA kits (eBioscience, San Diego).

96 wells plates were coated with 100 μL of capture antibodies (diluted in coating buffer to the concentration recommended by manufacturer's protocol) overnight at 4° C. Then, after washing the plates 5 times, 200 μL of assay diluents was added to each well and incubated for 1 hr at room temperature for blocking. After washing each well with wash buffer five times, cell culture sample or each cytokine standard protein sample was diluted and 100 μl of 25 each added into each well. The plate containing samples were incubated overnight at 4° C.

Then, after washing the plate five times with the wash buffer, 100 μl of secondary antibody conjugated to avidin was added and incubated for 1 hr at room temperature.

Following incubation with the secondary antibody, the plate was washed five times and incubated with 1001 μl of avidin-HRP (BD Bioscience) for 30 min at room temperature. After washing the plate seven times, 100 μl of TMB solution (Pierce) was added and incubated for 15 min at room temperature. The reaction was stopped by adding 50 μl of 2N H2S04 in each well The optical density at 450 nm was measured using a microplate reader. Statistical analysis was performed by variance analysis using ANOVA procedure of SPSS program, and verified the significance between analyses using Duncan's multiple range test.

### Experiment 2-3. IL-6 Secretion Measurement

As shown in Table 4 below, treatment with LPS alone increased the cytokine IL-6 (interleukin-6) secretion. However, co-treatment with LPS and PEP-1 showed a decrease in the level of the pro-inflammatory cytokine IL-6 secretion. More importantly, after the treatment with PEP-1, the level of pro-inflammatory cytokine secretion decreased by more 15 than 70%, which indicates a robust anti-inflammatory effect of Pep1.

TABLE 4
Cytokine IL-6 production inhibition by PEP-1

| | | NO Expression | Decreased NO |
|---|---|---|---|
| | | Level of | Expression |
| Test sample | | control (%) | Level (%) |
| PBS | | 0 | — |
| LPS 1 μg/mL | PBS | 100 | 0 |
| | PEP 1 (0.5 μg/mL) | 28 | 72 |

### Experiment 2-4. HMGB1. TN F-a. COX-2 Expression Inhibition

Protein expression level was determined by Western blot analysis. Cells grown in PEP-1 containing medium were washed with PBS, treated with 0.05% trypsin-EDTA, and collected by centrifugation. The collected cells were dissolved in an appropriate volume of lysis buffer. Intracellular debris was pelleted by centrifugation, and equal amount of protein from each sample was separated by SDS-polyacrylamide gel electrophoresis. The separated protein was transferred to nitrocellulose membrane (Schleicherand Schuell, Keene, N.H., USA), then was tested for the antibody specific for each protein. The membrane was incubated with ECL (enhanced chemiluminoesence) solution (Amersham Life Science Corp., Arlington Heights, Ill., USA), exposed to X-ray, and the level of protein expression was analyzed according to the exposure level shown on the X-ray film.

Western blot analysis was performed to determine the inhibitory effect of Ppep1 on the cytokine protein expression. As shown in Table 5 below, stimulation of cells with LPS increased the expression of cytokines; HMGB1, TNF-α and COX. However, if cells were treated with both LPS and Pep1, the expression level of pro-inflammatory cytokines mentioned above decreased. The result showing the treatment with Pep1 decreased pro-inflammatory cytokine levels by more than 70% provide strong evidence supporting the anti-inflammatory effect of Pep1.

TABLE 5
The measurement of inhibitory effect of Pep1 on
pro-inflammatory cytokine expression level.

| | | Cytokine Expression Level (band | | |
|---|---|---|---|---|
| | | intensity) % of control | | |
| Test sample | | HMGB1 | TNFα | COX-2 |
| PBS | | — | — | — |
| LPS 1 μg/mL | PBS | 100 | 100 | 100 |
| | PEP 1 (0.5 μg/mL) | 30 | 25 | 22 |

### Example 2

### TNF-α Inhibitory Effect of PEP RIA Series (Sequence No. 1 to 161) Peptides

Based on the results from Example 1 in which the SEQ ID NO: 1 (PEP1) has the TNF-α inhibitory effect, experiment using peptides SEQ ID NO: 1 to 161 were carried out to confirm their TN F-a inhibitory effect. The synthesis of peptides SEQ ID NO: 1 to 161 used the same method mentioned above in Example 1 (method used for synthesis of PEP1), but the amino acids added were different.

### Experiment 1: Cell Culture

PBMC (peripheral blood mononuclear cells) layer was separated from blood samples (50 ml) collected in healthy subjects using Biocoll Separating Solution (Biochrom AG, Berlin, Germany). The collected PBMC were enriched in RPMI 1640 medium containing 20% human serum for 30 mins, and then transferred to 100-mm polystyrene cell culture plate coated with human serum for incubation for 2 hrs at 37° C., 5% CO₂ incubator. Monocytes were detached from the bottom of the plate using cold PBS, and incubated to reach the number of 1×10⁵ cell/well in 96-well plate with RPMI 1640 medium (supplemented with penicillin-S streptomycin; 100 mg/ml, human serum; 20%) overnight.

### Experiment 2: Analysis of TN F-a Inhibitory Effect in PBMC-Derived Monocyte

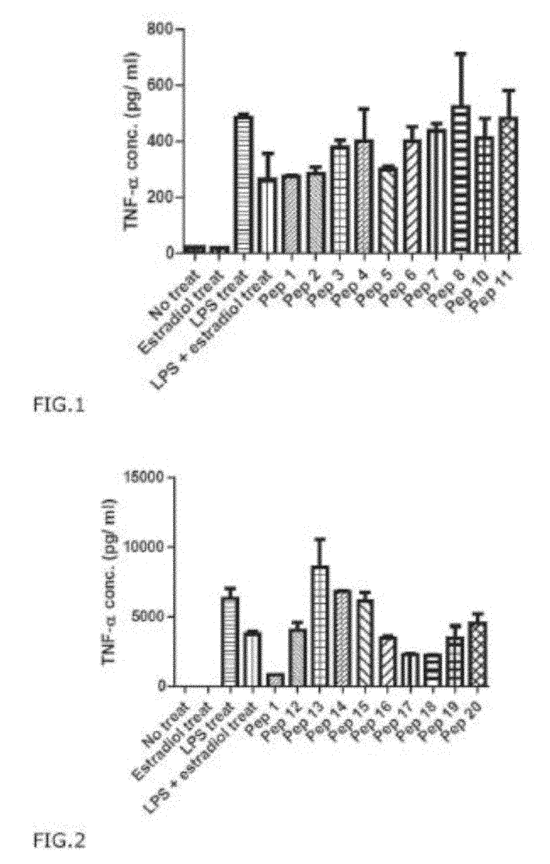

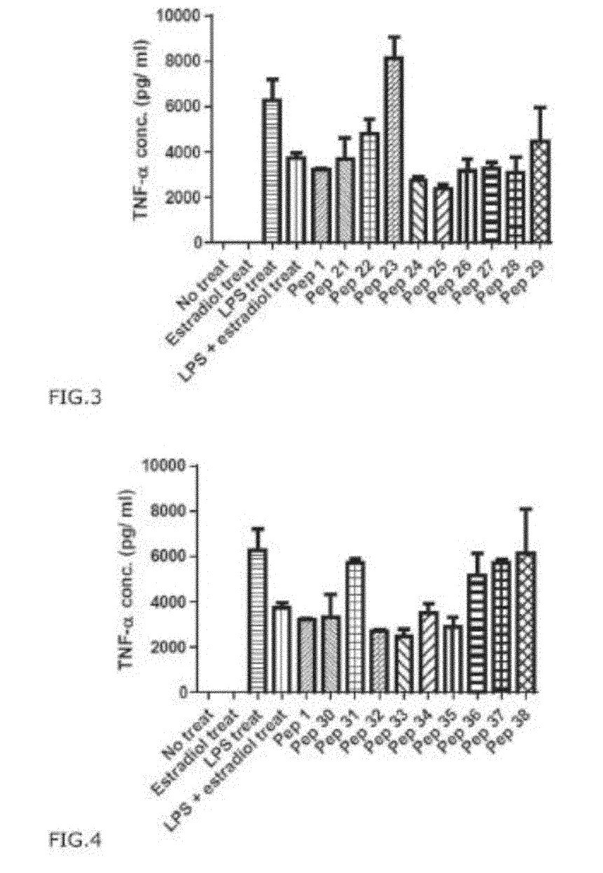

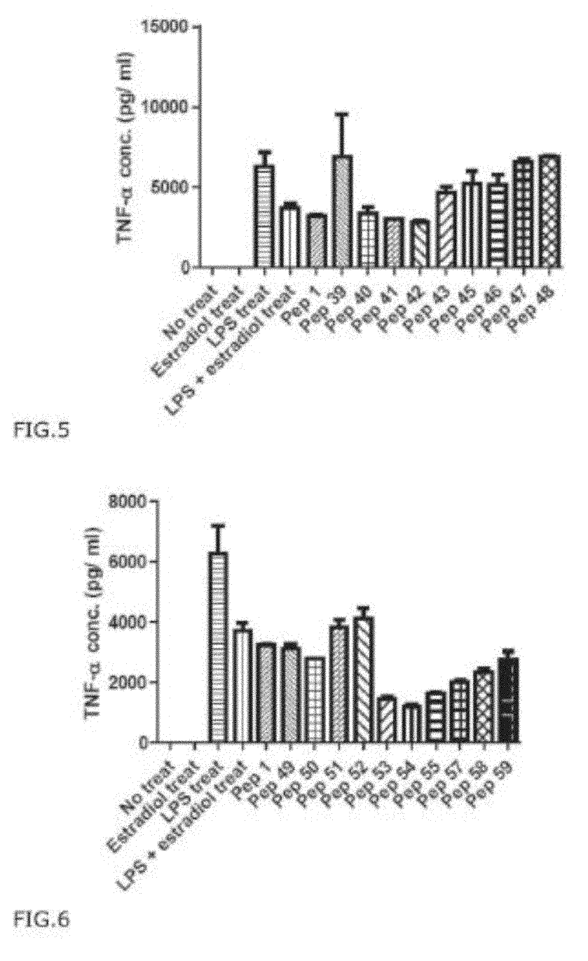

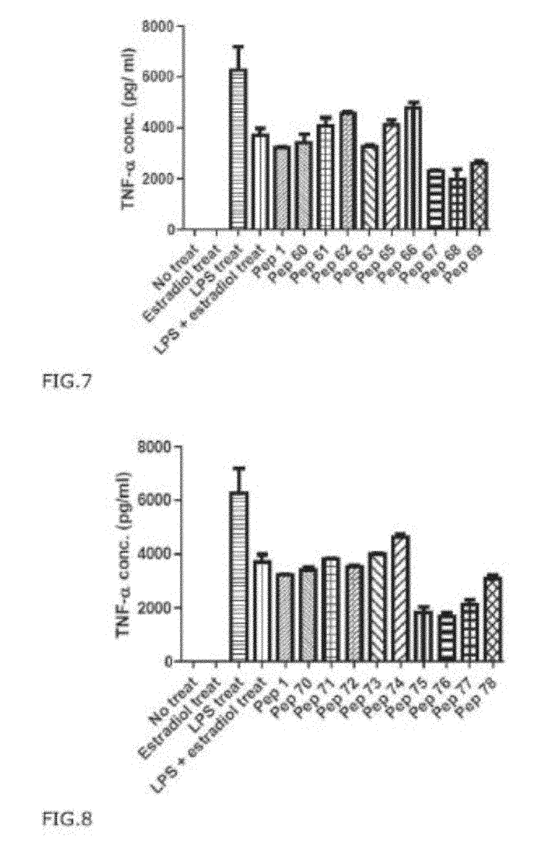

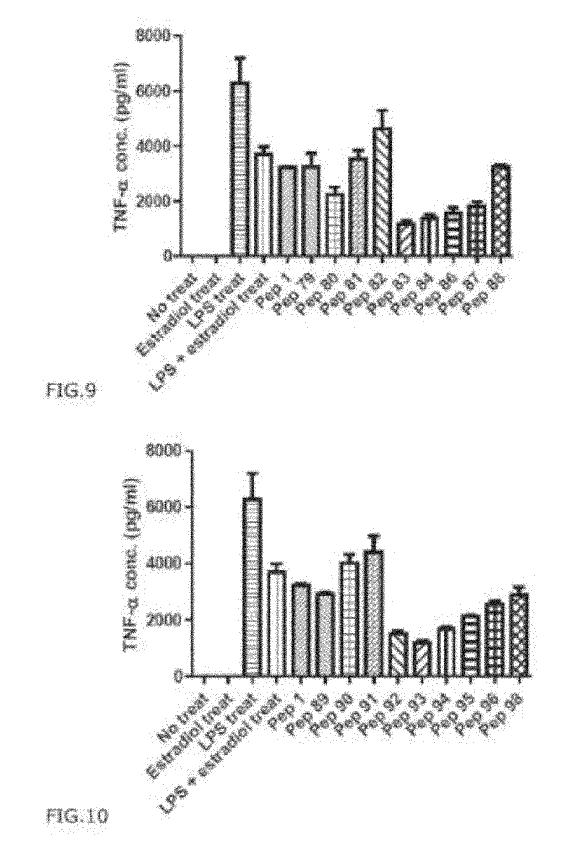

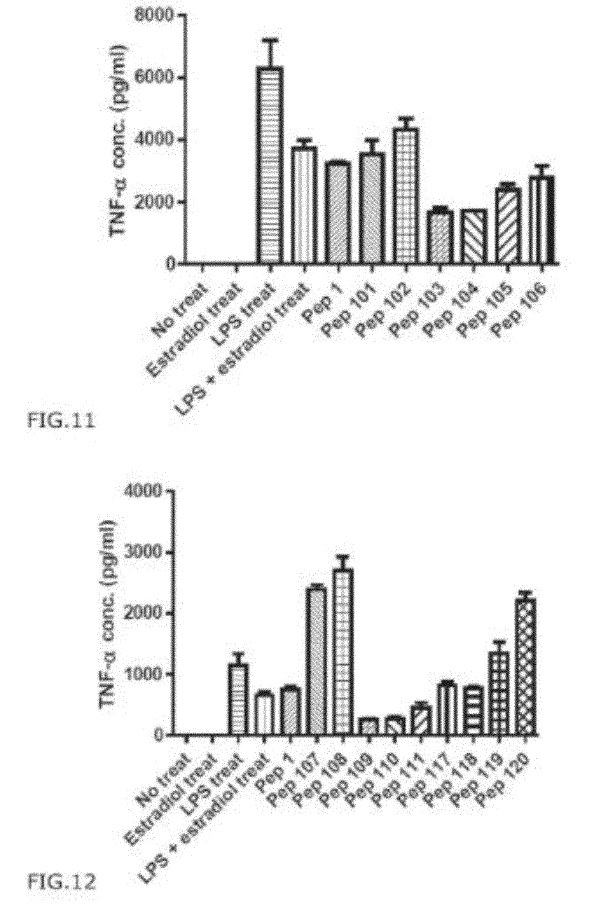

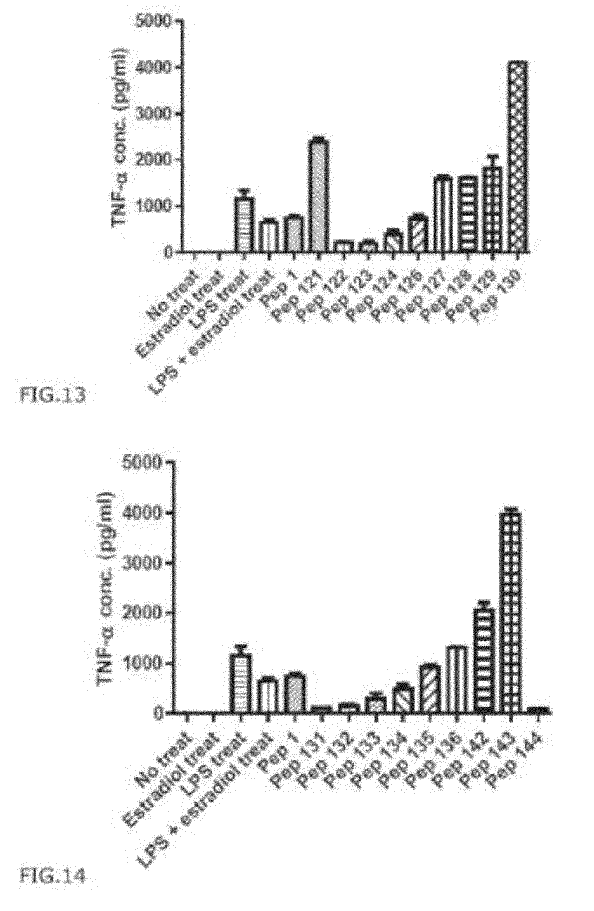

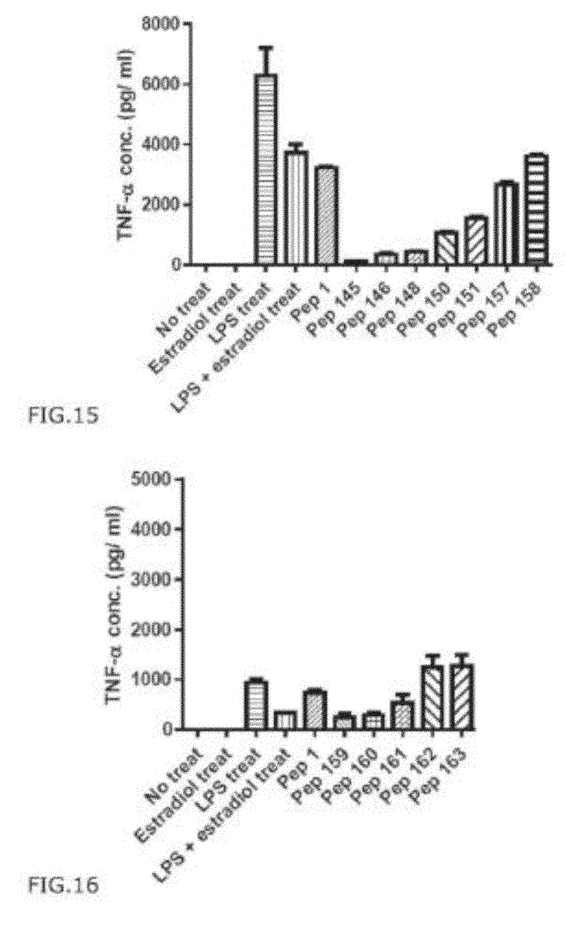

ELISA was performed to find out how the peptides of the PEP RIA series influence TNF-α level. PBMC-derived monocytes were incubated to reach the number of 1×105 cells per well in a 96-well plate and then treated with LPS (lipopolysaccharide; 10 ng/ml, Sigma) for 2 hours. To the monocytes that were washed three times with PBS, OPTI-MEM culture medium was added to induce cell starvation for an hour, 4 μM of the peptide was taken out and incubated for 2 hours. There were three negative control groups. The first group was not treated with anything. The second group that was treated with estrogen (in this experiment, estradiol was used as a kind of estrogen). The third group was treated with LPS (10 ng/ml) or with LPS (10 ng/ml) as well as estrogen (20 nM). PEP1 that was confirmed to have TNF-α inhibiting activity was used as a positive control to measure TNF-α inhibiting activity. After incubation, TNF-α was measured by following the ELISA kit manual (R&D, Minneapolis, Minn., USA). The details of quantification method can be found in Experiment 2.2 of Example 1. Using the method stated above, Peptides with TNF-α inhibiting effect were screened. PBMC-derived monocytes were stimulated with LPS (10 ng/ml), which is endotoxin, for 2 hours and were induced to starve by adding OPTI-MEM for 1 hour. After that, 4 μM of 161 peptides were treated and incubated for 2 hrs. The amount of TNF-α in the cell culture medium was measured using ELISA, and the peptides with TNF-α inhibiting effect were screened by comparing to the negative and positive controls (FIG. 1 to FIG. 16).

The followings are the peptides that showed TNF-α inhibiting effect when compared to the control group that was treated with only LPS: SEQ ID NO: 1 to SEQ ID NO: 6, SEQ ID NO: 9, SEQ ID NO: 11, SEQ ID NO: 14 to 21, SEQ ID NO: 23 to SEQ ID NO: 37, SEQ ID NO: 39 to SEQ ID NO: 44, SEQ ID NO: 47 to SEQ ID NO: 53, SEQ ID NO: 55 to SEQ ID NO: 61, SEQ ID NO: 63 to SEQ ID NO: 82, SEQ ID NO: 84 to SEQ ID NO: 94, SEQ ID NO: 96, SEQ ID NO: 99 to SEQ ID NO: 104, SEQ ID NO: 107 to SEQ ID NO: 109, SEQ ID NO: 115, SEQ ID NO: 116, SEQ ID NO: 120 to SEQ ID NO: 122, SEQ ID NO: 124, SEQ ID NO: 129 to SEQ ID NO: 133, SEQ ID NO: 142 to SEQ ID NO: 144, SEQ ID NO: 146, SEQ ID NO: 148, SEQ ID NO: 149, and SEQ ID NO: 155 to SEQ ID NO: 159.

Also, the followings are the peptides that showed TNF-α inhibiting effect when compared to the group treated with LPS and estrogen: SEQ ID NO: 15 to SEQ ID NO: 18, SEQ ID NO: 23 to SEQ ID NO: 27, SEQ ID NO: 29, SEQ ID NO: 31 to SEQ ID NO: 34, SEQ ID NO: 39 to SEQ ID NO: 41, SEQ ID NO: 47, SEQ ID NO:48, SEQ ID NO: 51 to SEQ ID NO: 53, SEQ ID NO: 55 to SEQ ID NO: 58, SEQ ID NO: 61, SEQ ID NO: 65 to SEQ ID NO: 68, SEQ ID NO: 70, SEQ ID NO: 73 to SEQ ID NO: 79, SEQ ID NO: 81, SEQ ID NO: 82, SEQ ID NO: 84 to SEQ ID NO: 87, SEQ ID NO: 8=90 to SEQ ID NO: 94, SEQ ID NO: 96, SEQ ID NO: 99, SEQ ID NO: 101 to SEQ ID NO: 104, SEQ ID NO: 107 to SEQ ID NO: 109, SEQ ID NO: 120, SEQ ID NO: 121, SEQ ID NO: 129 to SEQ ID NO: 132, SEQ ID NO: 142 to SEQ ID NO: 144, SEQ ID NO: 146, SEQ ID NO: 148, SEQ ID NO: 149, and SEQ ID NO: 157 to SEQ ID NO: 159.

### Experiment 3: Analysis of Peptides Affecting TNF-α Level in THP1 Cell Line

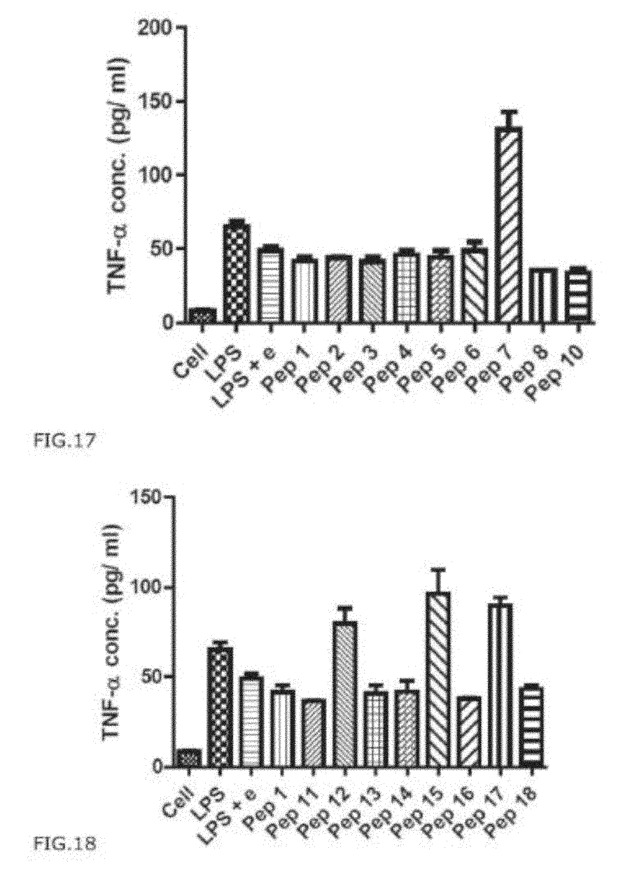

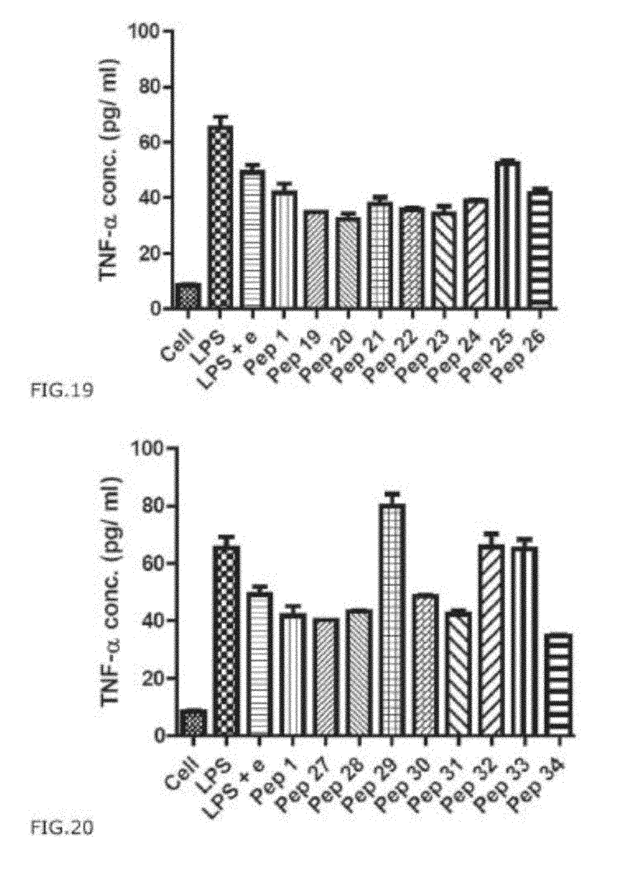

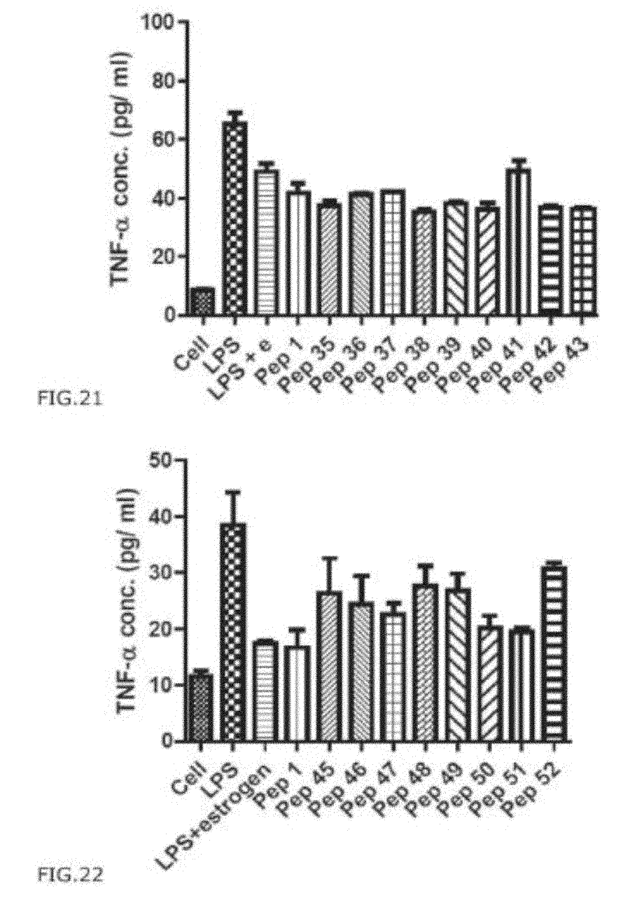

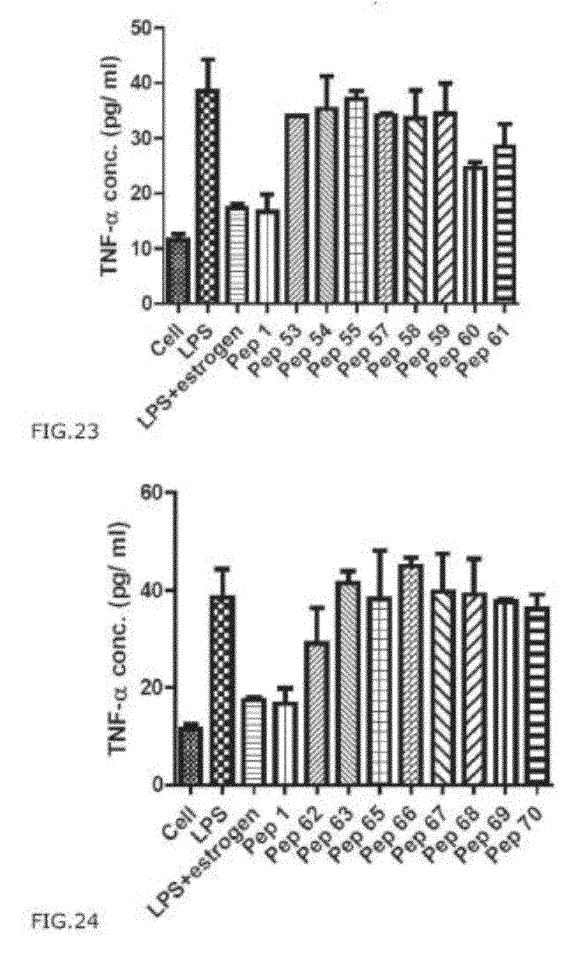

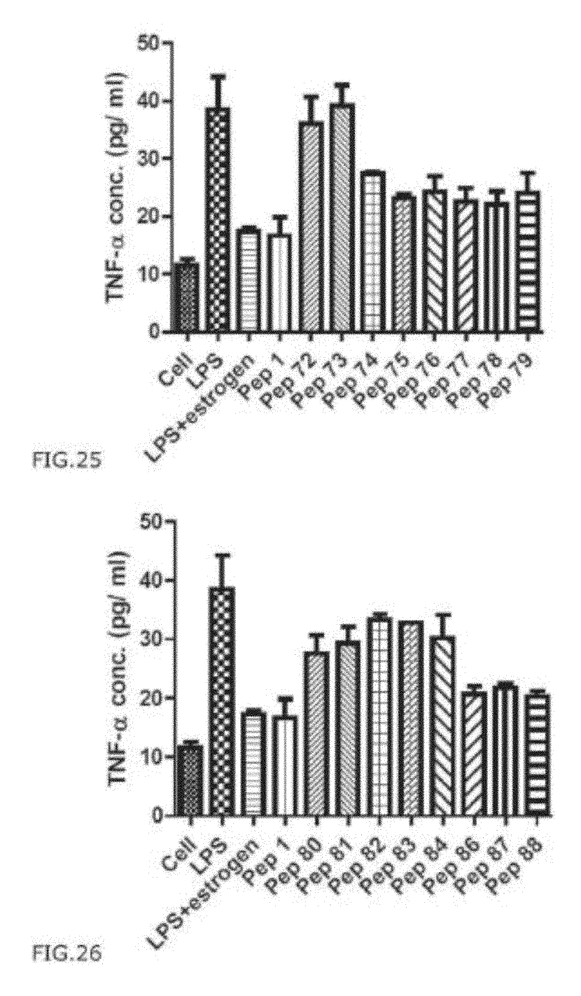

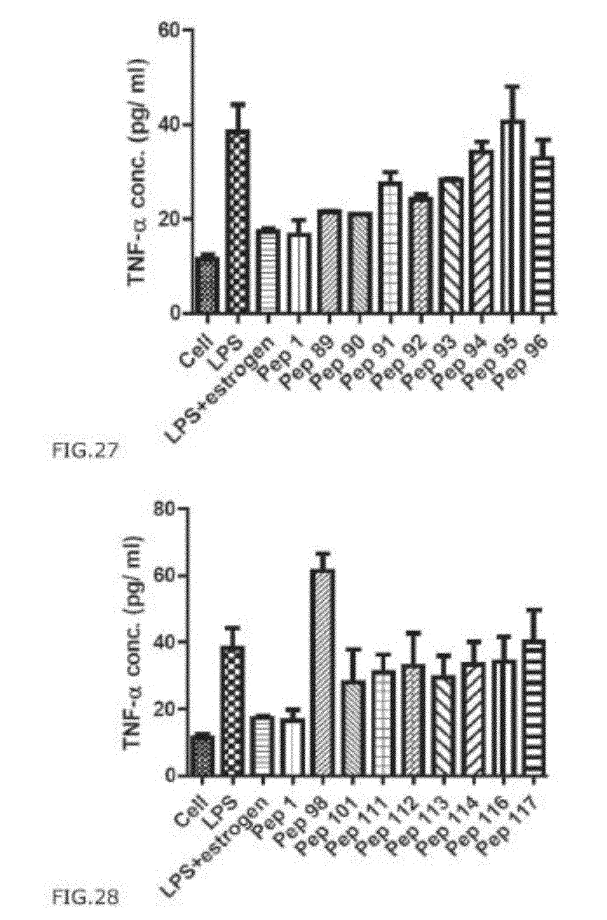

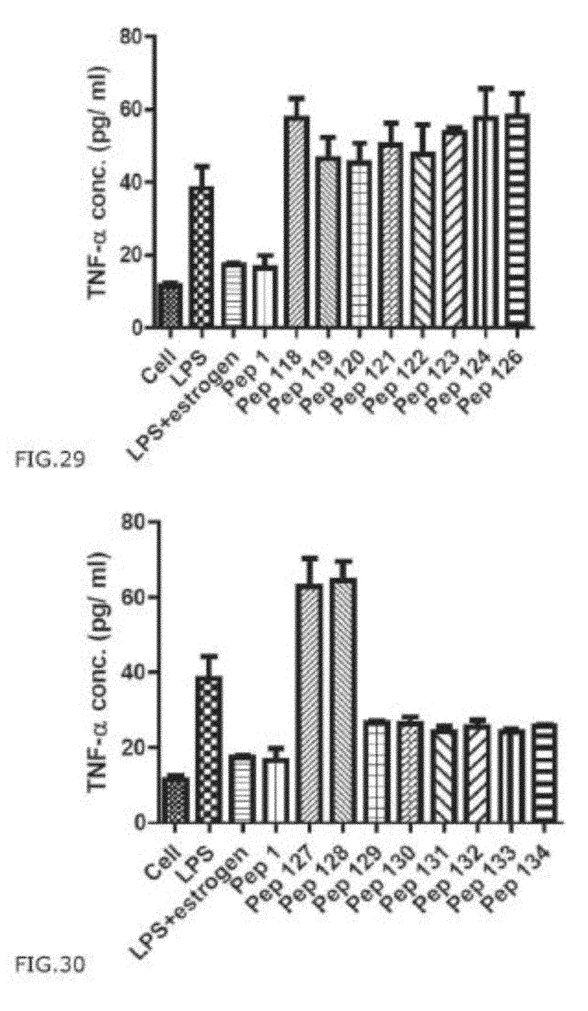

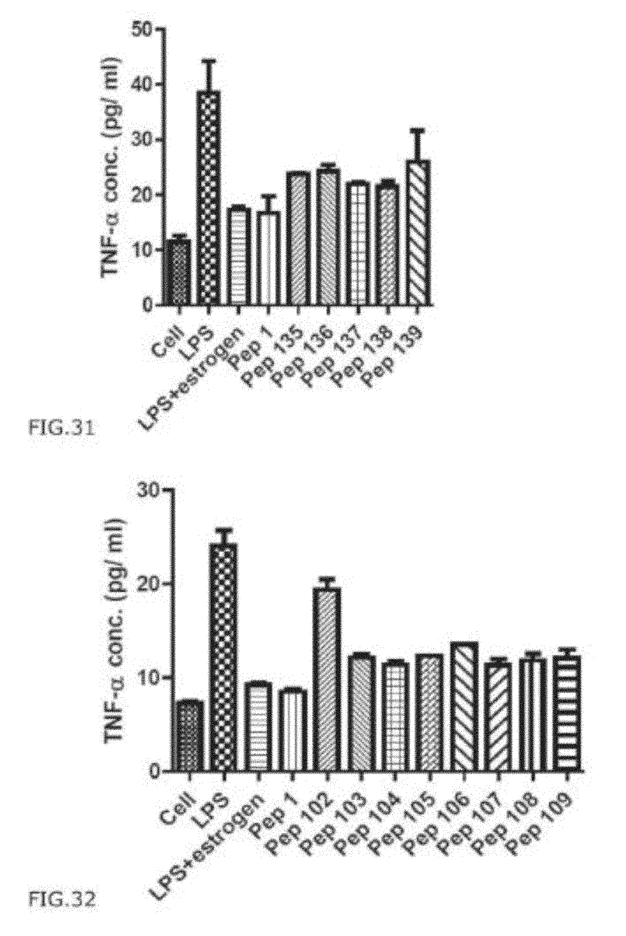

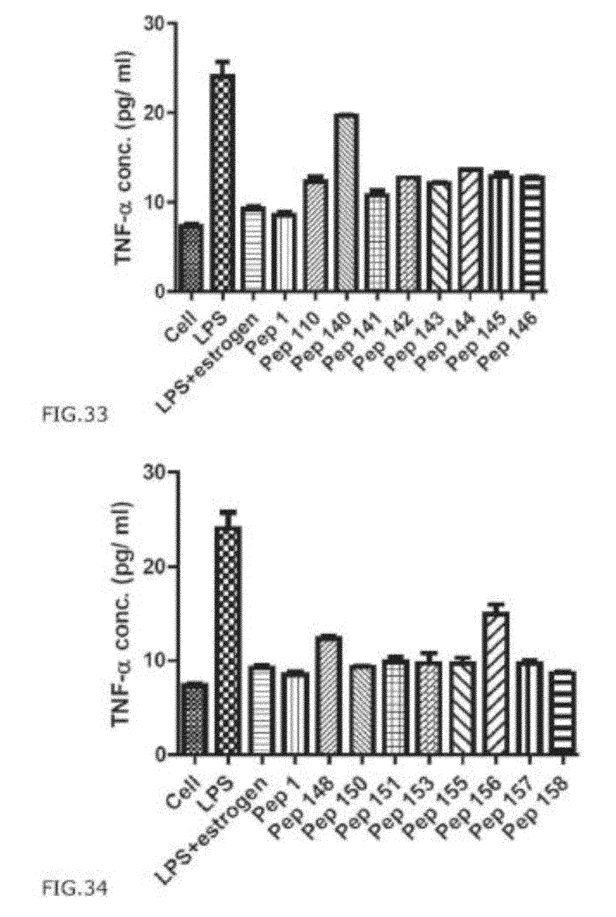

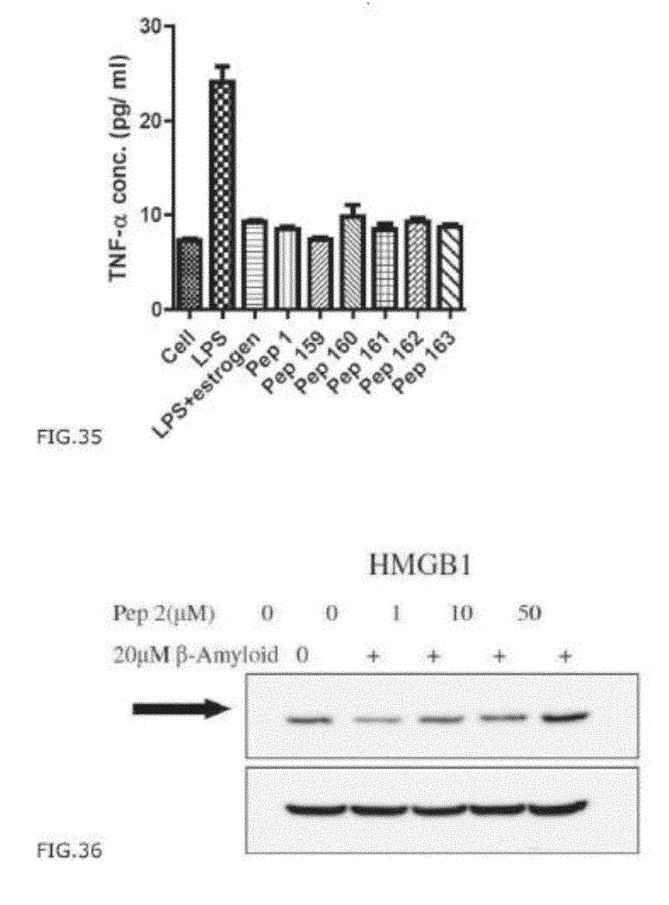

Experiment was carried out using THP-1 cell line (American Type Culture Collection (ATCC), Manassas, Va., USA) which is human acute monocytic leukemia. THP-1 cells were incubated to reach the number of 1×105 cells per well in a 96-well plate 15 with RPMI 1640 medium for 24 hrs, followed by addition of 1001JM of PMA (phorbol 12-myristate 13-acetate) for the differentiation into macrophage. After differentiation of THP-1 into macrophage by PMA for a day, LPS was treated for 2 hrs and washed off. Starvation for an hour and PEP1 treatment followed. THP-1 cell differentiated by PMA was treated with LPS (lipopolysaccharide; 10 ng/ml, Sigma) for 2 hours, followed by 2 times washes with PBS. To the cells, OPTI-MEM culture medium was added to induce cell starvation for an hour, and 1 μM of 161 peptides was taken out and incubated for an hour. After incubation, TNF-α level was measured by using the ELISA kit and the peptides which reduce the TNF-α level were screened (FIG. 17 to FIG. 35).

As a result, peptide SEQ ID NO: 1 to SEQ ID NO: 5, SEQ ID NO: 7, SEQ ID NO: 9, SEQ ID NO: 10, SEQ ID NO: 12, SEQ ID NO: 13, SEQ ID NO: 15, SEQ ID NO: 17 to SEQ ID NO: 27, SEQ ID NO: 29, SEQ ID NO: 30, SEQ ID NO: 32 to SEQ ID NO: 53, SEQ ID NO: 55 to SEQ ID NO: 60, SEQ ID NO: 67, SEQ ID NO: 68, SEQ ID NO: 70, SEQ ID NO: 72 to SEQ ID NO: 82, SEQ ID NO: 84 to SEQ ID NO: 92, SEQ ID NO: 94, SEQ ID NO: 99 to SEQ ID NO: 112, SEQ ID NO: 114, SEQ ID NO: 127 to SEQ ID NO: 144, SEQ ID NO: 146, SEQ ID NO: 148, SEQ ID NO: 149, SEQ ID NO: 151 and SEQ ID NO: 153 to SEQ ID NO: 161 appeared to reduce the TNF-α level compared to control group treated only with LPS.

In addition, SEQ ID NO: 1 to SEQ ID NO: 5, SEQ ID NO: 7, SEQ ID NO: 9, SEQ ID NO: 10, SEQ ID NO: 12, SEQ ID NO: 13, SEQ ID NO: 15, SEQ ID NO: 17 to SEQ ID NO: 23, SEQ ID NO: 25 to SEQ ID NO: 27, SEQ ID NO: 29, SEQ ID NO: 30, SEQ ID NO: 33 to SEQ ID NO: 43, SEQ ID NO: 156, SEQ ID NO: 157 and SEQ ID NO: 159 were selected as peptides which reduce the expression level of TNF-o compared to that of group treated with LPS and estrogen.

### Example 3

### Effects of Peptides (SEQ ID No: 1 to 161) on Inflammation by Amyloid-β Protein

HMGB1 first undergoes acetylation and translocation to cytoplasm by external stimulation. Then it is secreted out of the cell, therefore serving the role of inflammation-causing cytokine. Because when one has an inflammation due to such activity, HMGB1 protein is secreted from the cell, and patients with inflammatory diseases such as Churg strauss syndrome, rheumatoid arthritis and Sjogren's syndrome will present with elevated serum levels of HMGB1. Hence, if nucleus contains large amount of HMGB1 even when there is a stimulus that causes inflammation, it is suggestive of the fact that HMGB1 is not being secreted out of the cell, which means inflammation is being suppressed.

### Experiment 1. Cell Culture

Undifferentiated PC12 cells (ATCC, Rockville, Md., USA) were maintained in ogarithmic-phase growth on poly-1-lysine (Sigma, Saint Louis, Mo., USA)—precoated 100 mm dishes (Corning, Pa., USA) in RPMI 1640 medium (GIBCO, Grand Island, N.Y., USA) containing 10% heat-inactivated horse serum, 5% heat-inactivated fetal bovine serum, 100 units/ml penicillin, and 100 g/ml streptomycin. Cultures were incubated at 37° C. in a humidified atmosphere with 5% C02. The cultures were grown to 50% confluence and were harvested in Ca2+/Mg2+-free Hank's balanced salt solution containing 1 mM EDTA. Cells were plated at a density of 1×106 cells/100 mm dish and incubated for 24 hrs. For neuronal differentiation, PC12 cells were serum-starved for 12 hrs (RPMI1640 medium containing 100 units/ml penicillin and 100 g/ml streptomycin without horse serum or fetal bovine serum); thereafter, the cells were maintained in serum-free medium. After two days the medium was replaced with fresh serum-free medium. On day three, NGF (50 ng/ml, Sigma, Saint Louis, Mo., USA) was added to the medium, and the cultures were maintained for an additional three days. After differentiation, nPC12 cells were incubated with 201JM amyloid-β with several concentrations of the peptides [0 (control), 1, 10, and 50 μM] for 48 hrs.

### Experiment 2. Western Blot Analysis

Levels of HMGB1 were analyzed by western blotting. Briefly, 5×106 cells were washed twice in cold PBS, incubated for 10 min on ice in lysis buffer [50 mM Tris (pH 8.0), 150 mM NaCl, 0.02% sodium azide, 0.2% SDS, 100 μg/ml phenyl methyl sulfonyl fluoride (PMSF), 50 μI/ml aprotinin, 1% Igepal 630, 100 mM NaF, 0.5% sodium deoxy choate, 0.5 mM EDTA, 0.1 mM EGTA]; unbroken cells and nuclei were pelleted by centrifugation for 10 min at 2000×g and the lysates were cleared by centrifugation at 10,000×g. The antibodies used were: anti-HMGB1 (1:1000, Cell Signaling, Beverly, Mass., USA) and anti-13-tubulin (1:1000, Cell Signaling, Beverly, Mass., USA). The membranes were washed with Tris-buffered saline containing 0.05% Tween-20 (TBST), and then processed using HRP-conjugated anti-rabbit antibody (Amersham Pharmacia Biotech, Piscataway, N.J., USA) followed by ECL detection (Amersham Pharmacia Biotech,). The blots were quantified with an image analyzer (GE Healthcare, ImageQuant LAS 4000).

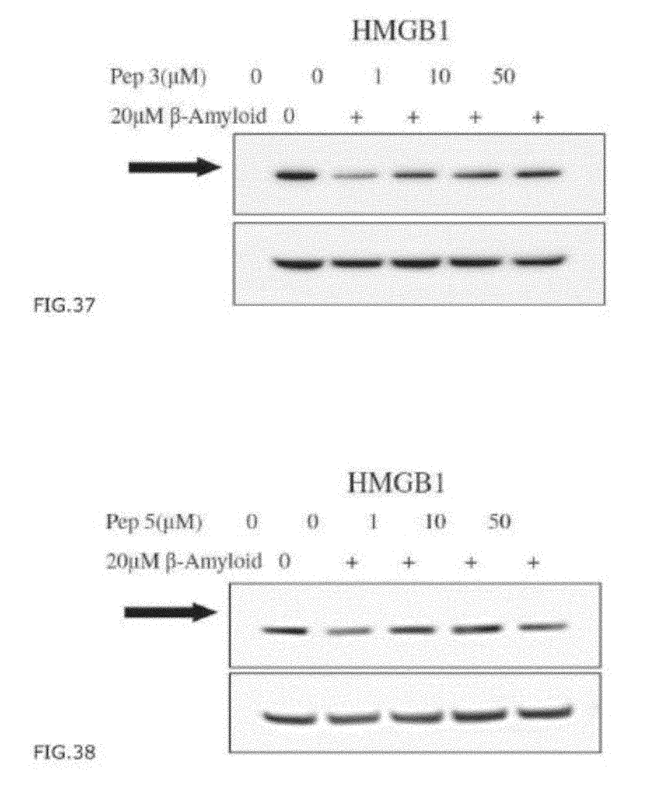

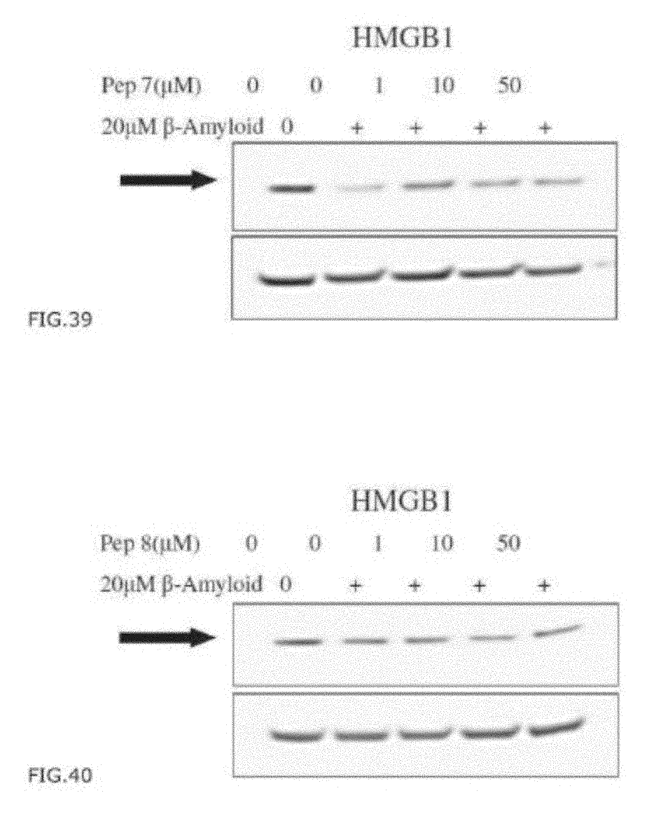

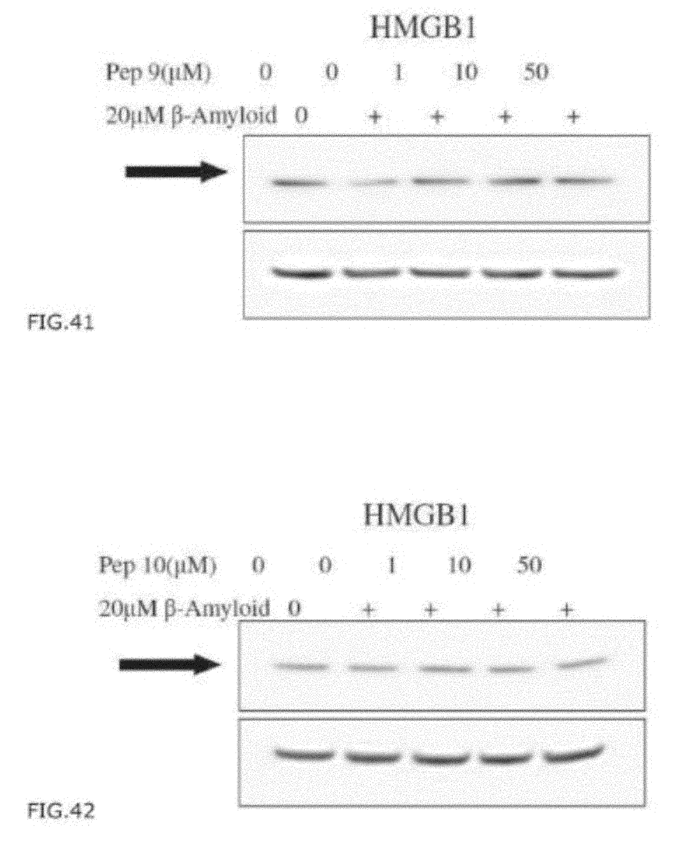

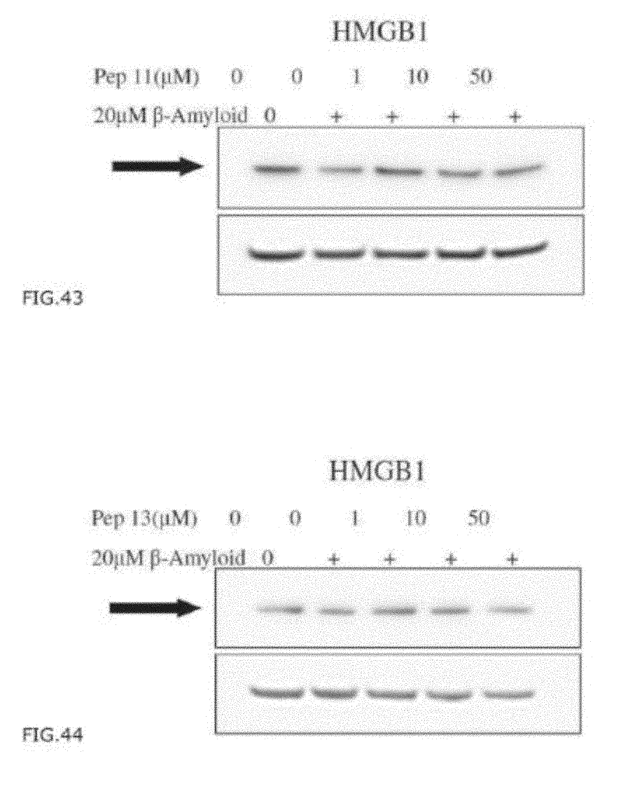

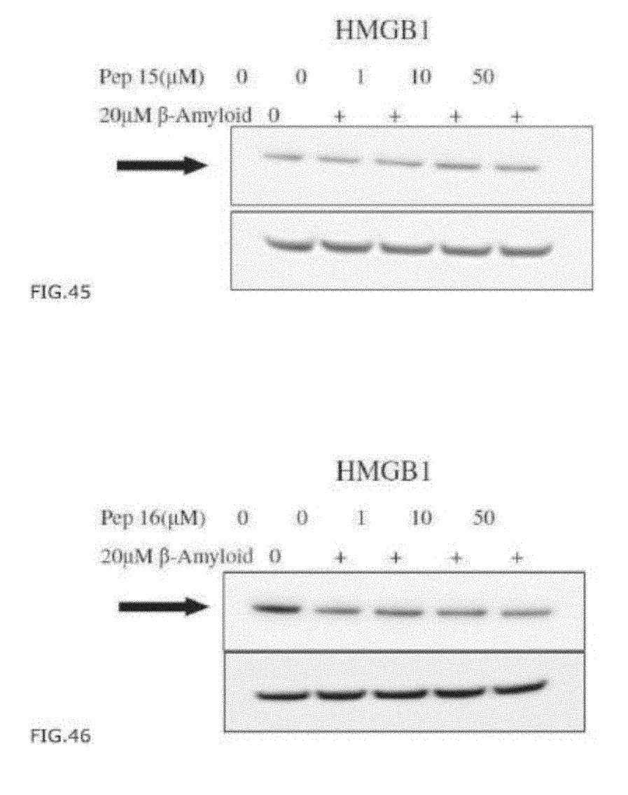

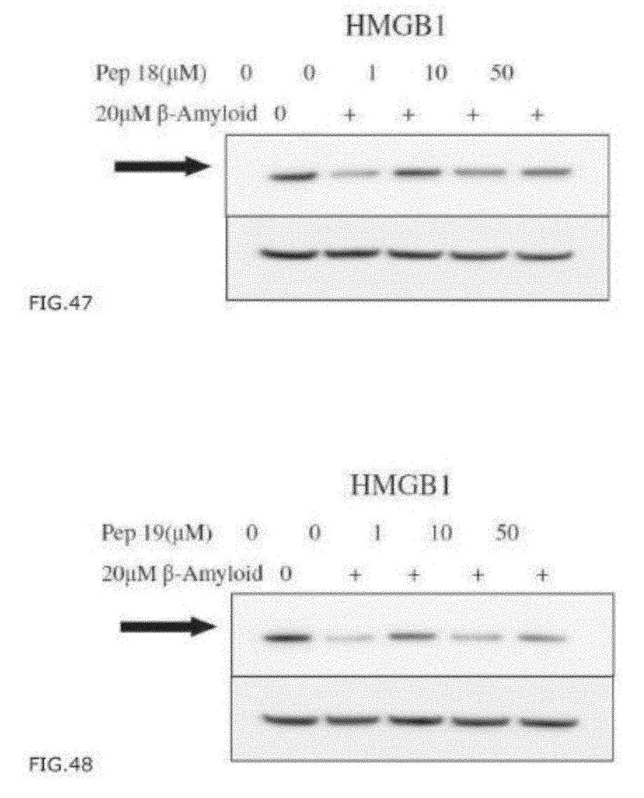

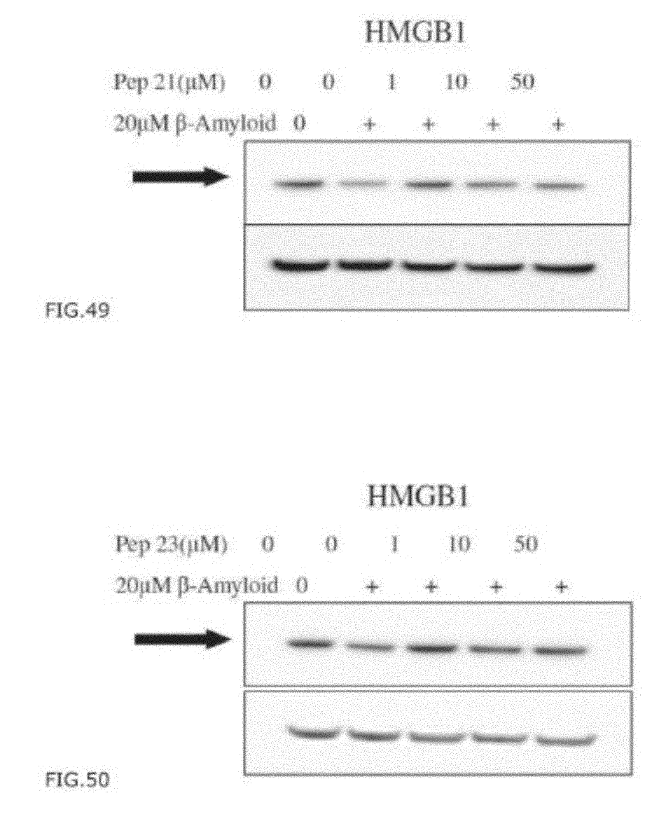

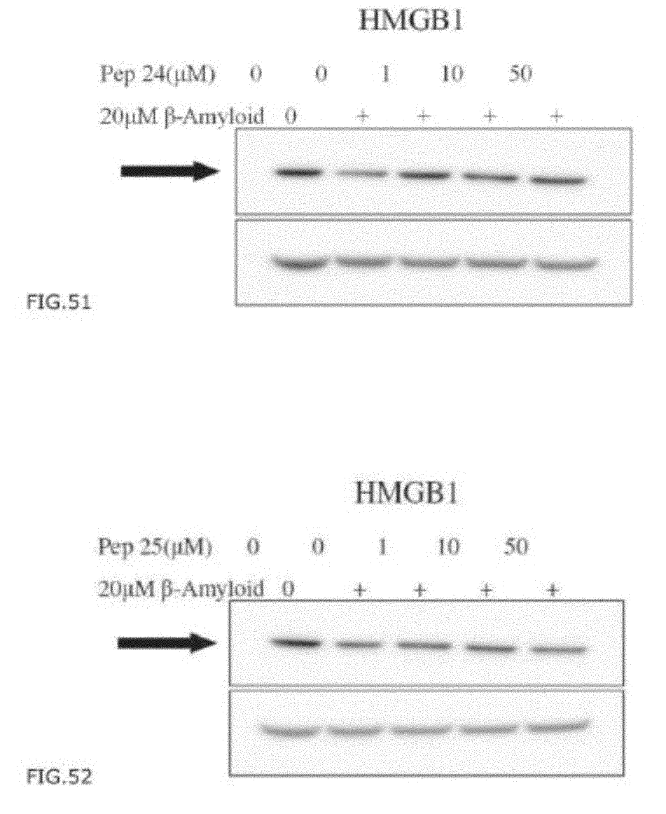

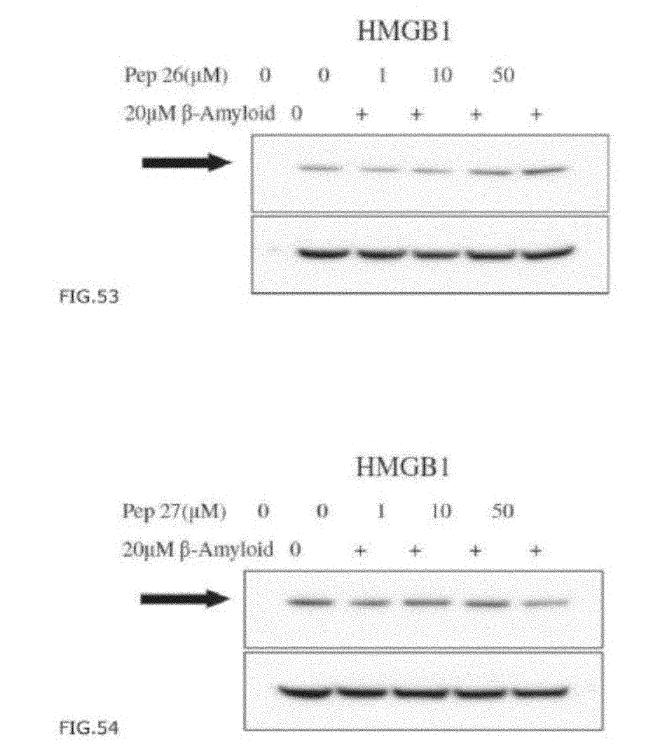

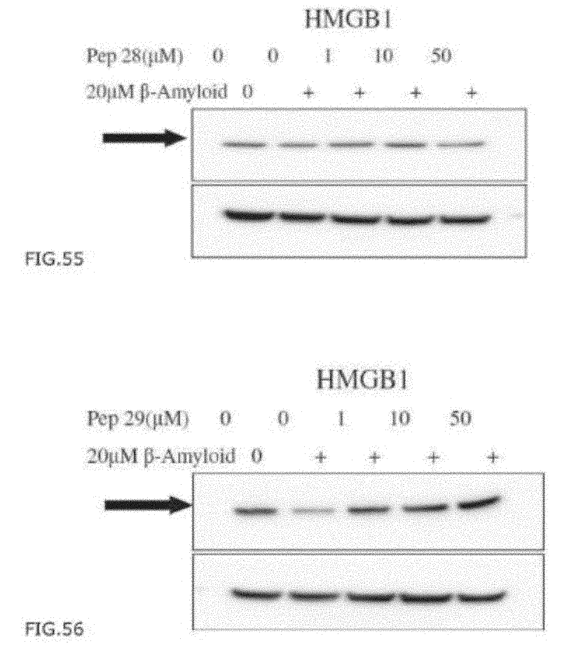

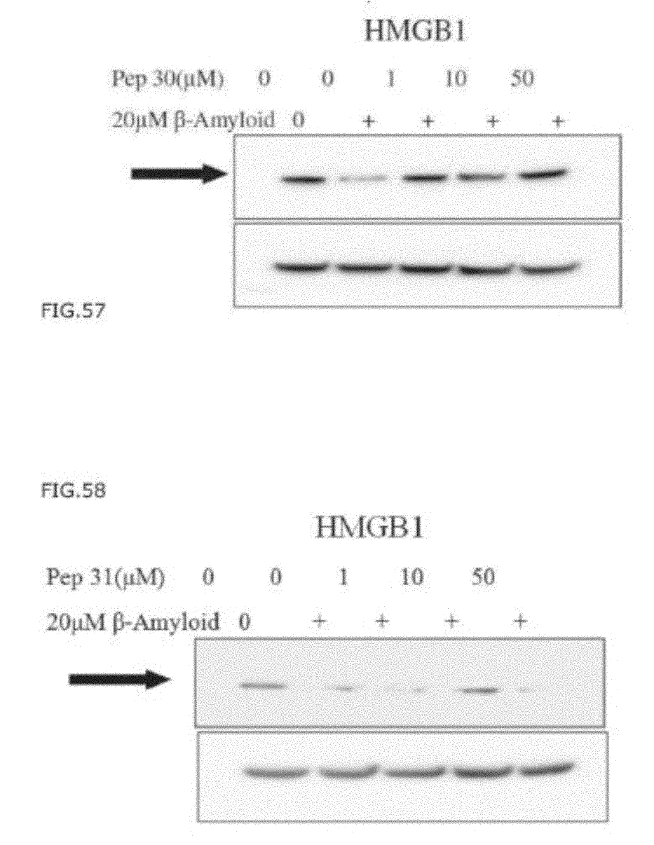

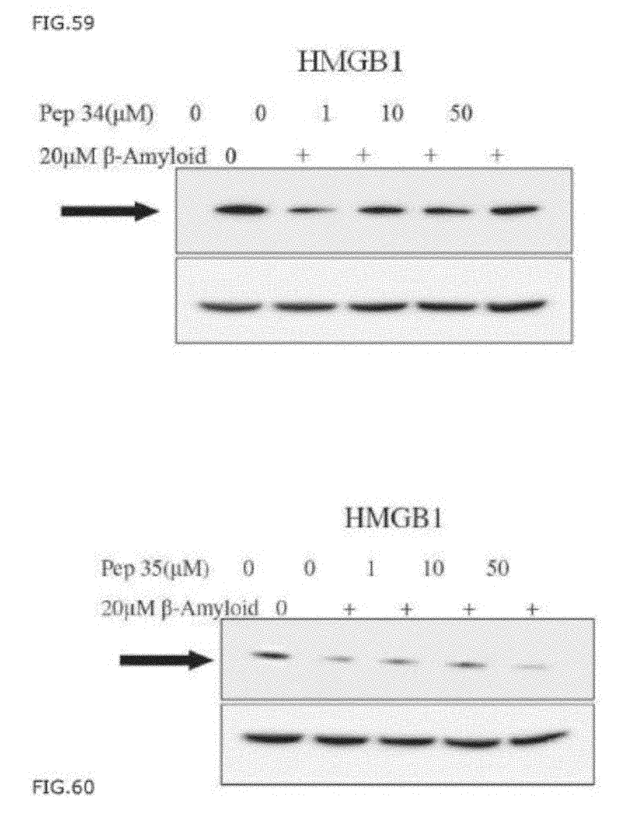

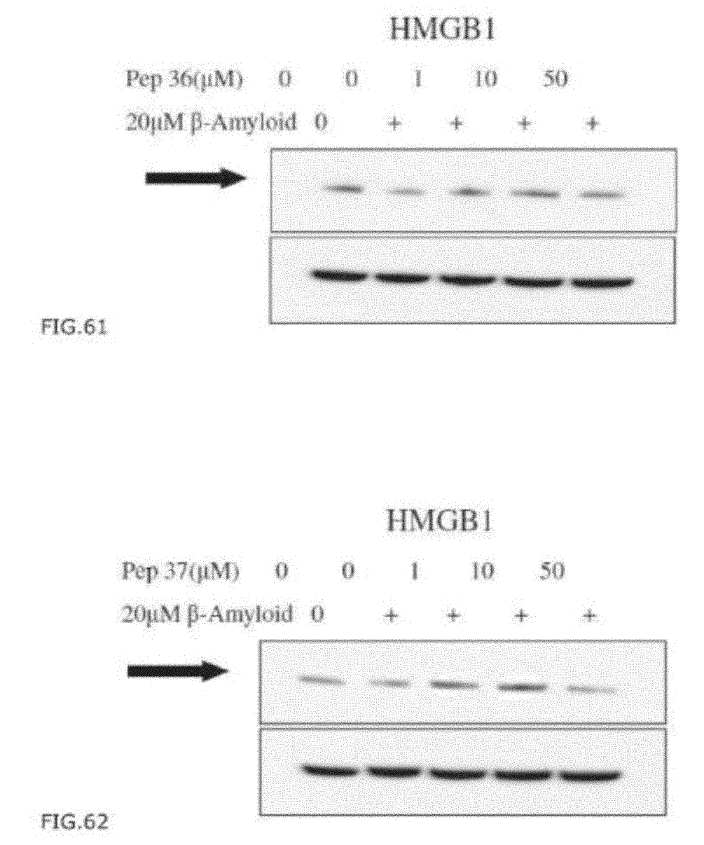

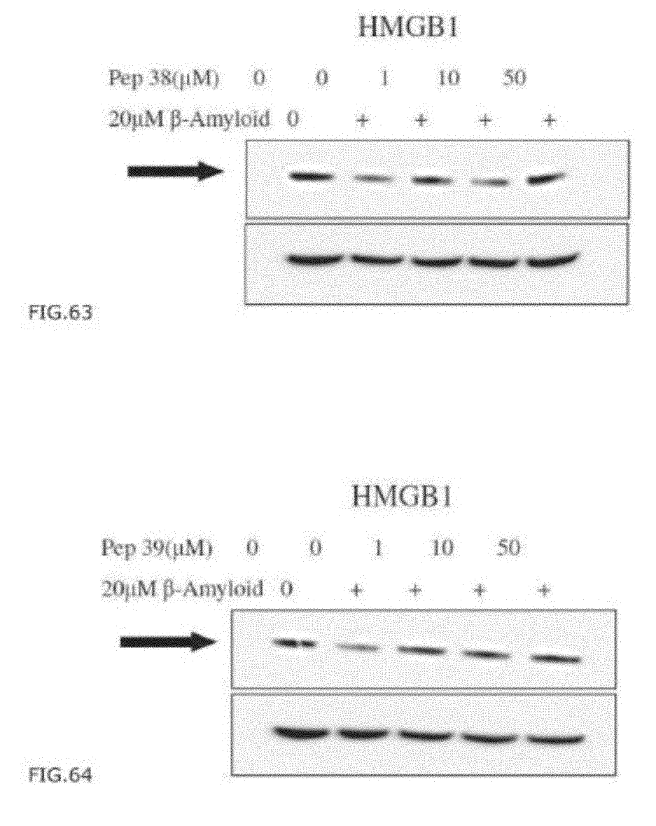

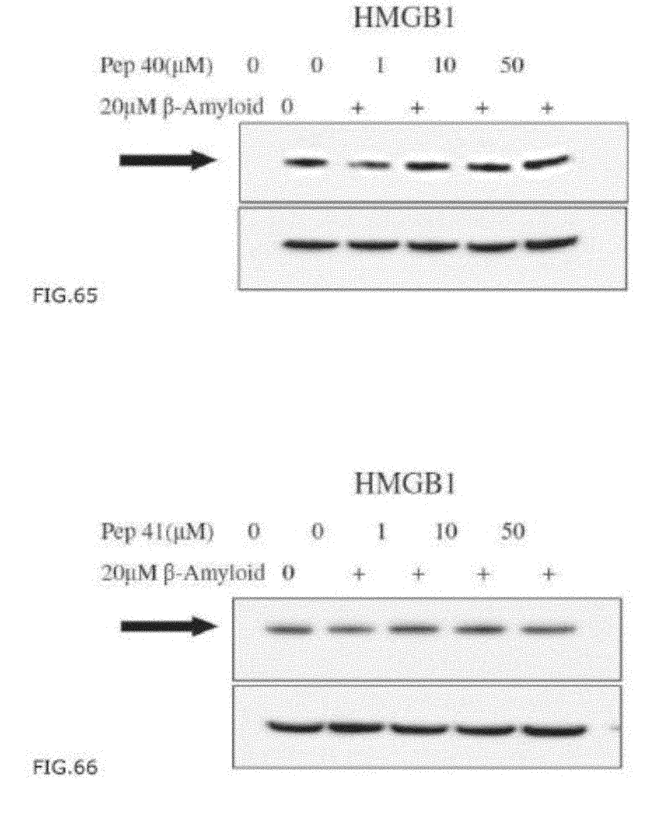

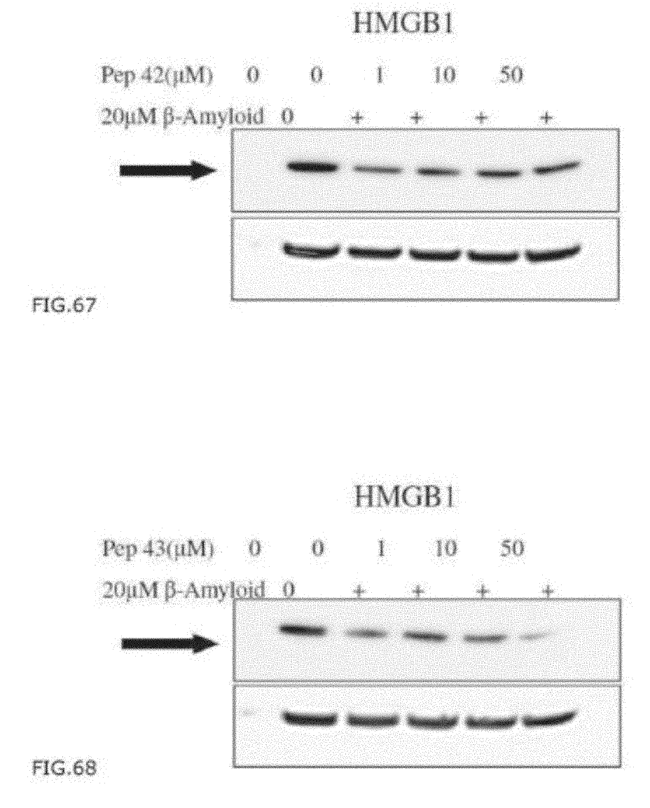

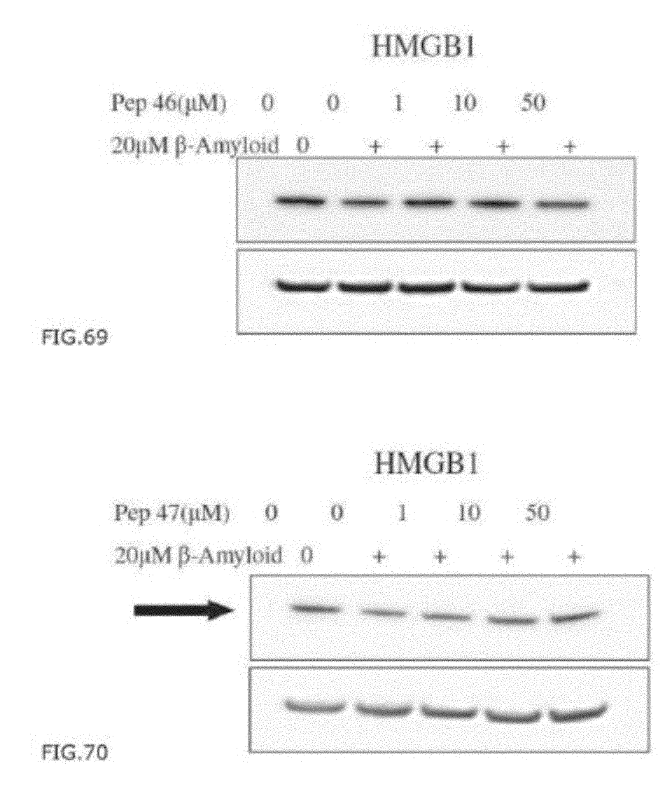

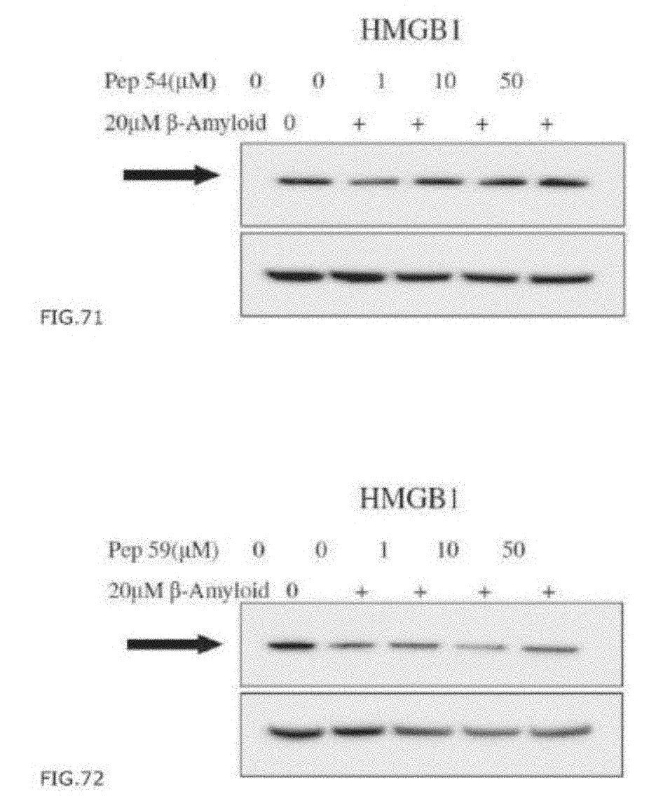

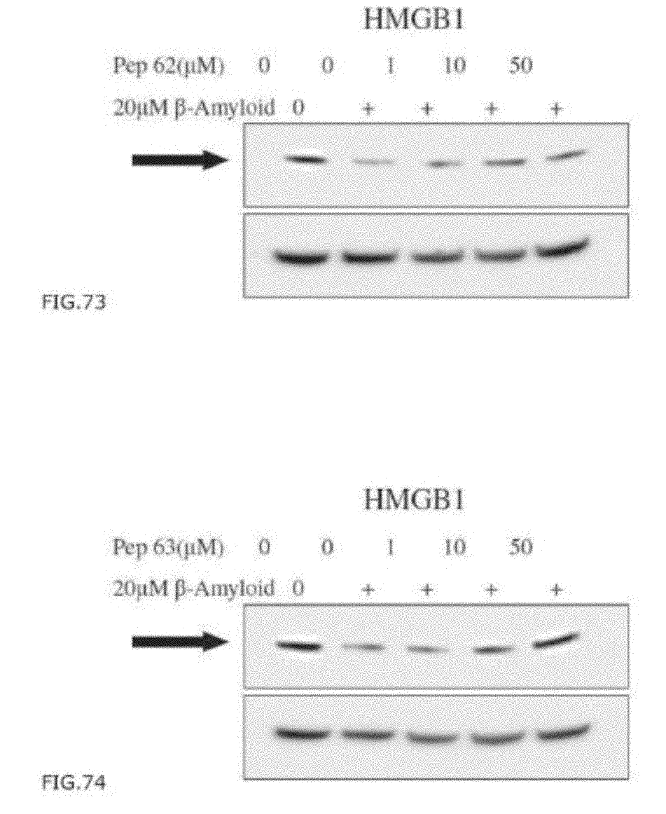

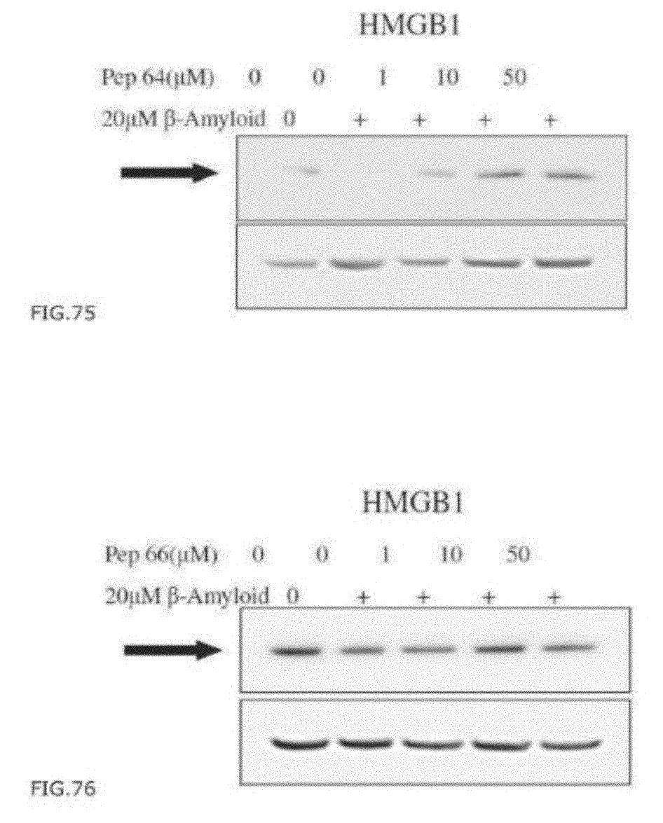

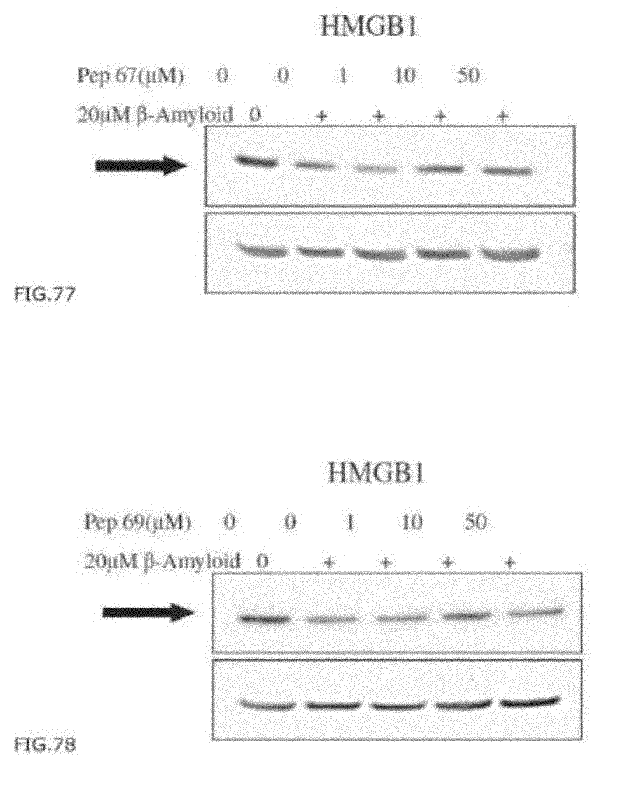

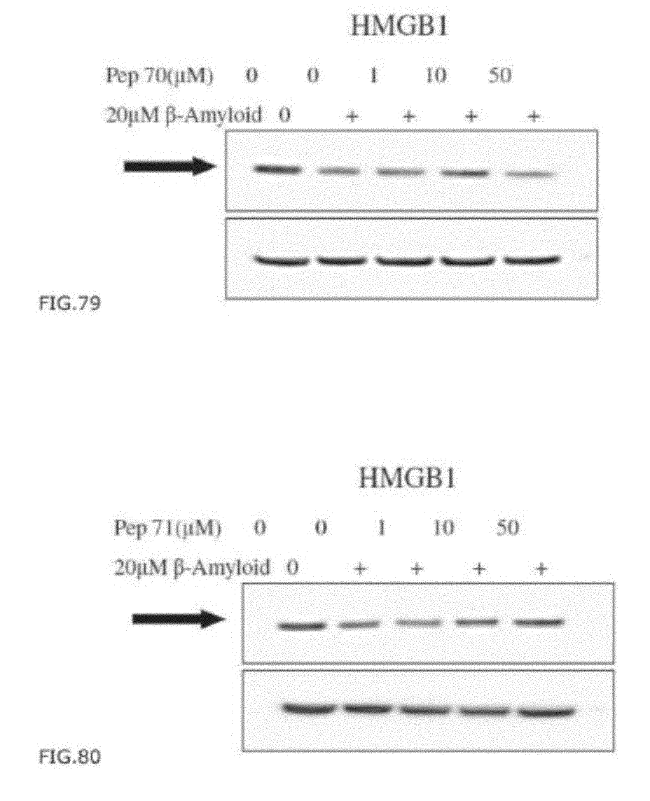

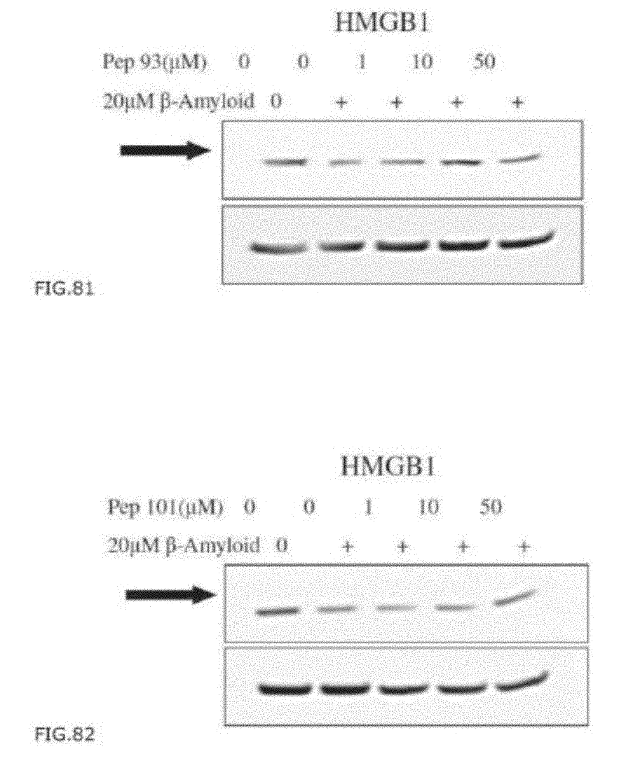

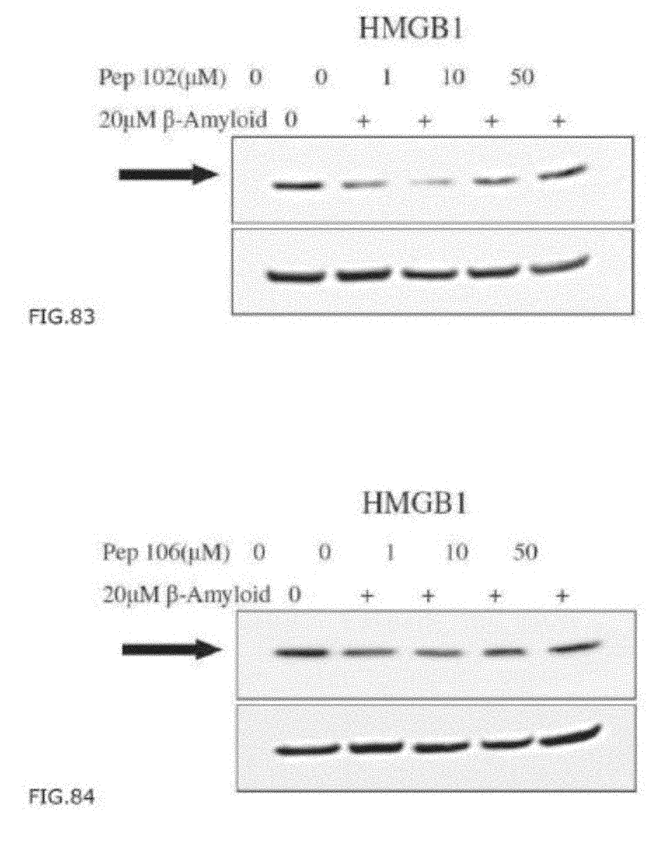

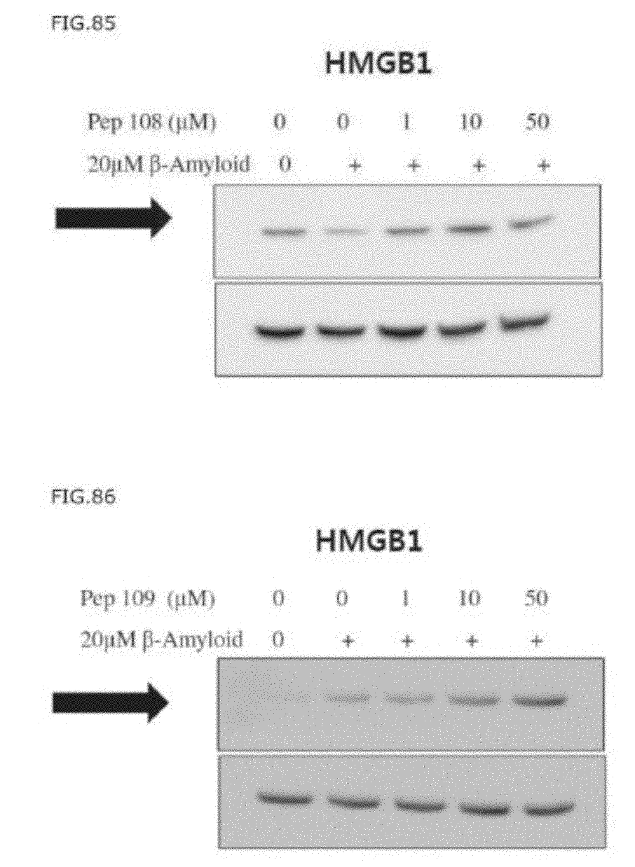

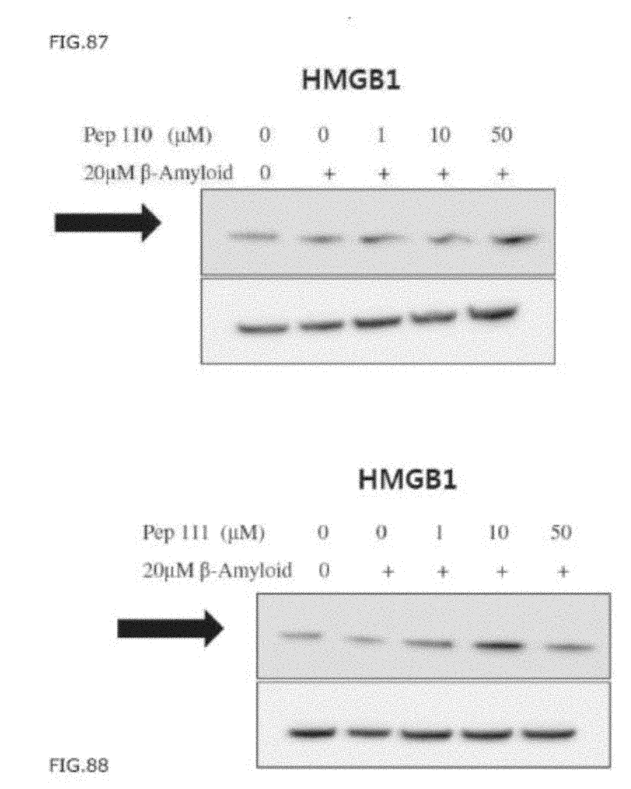

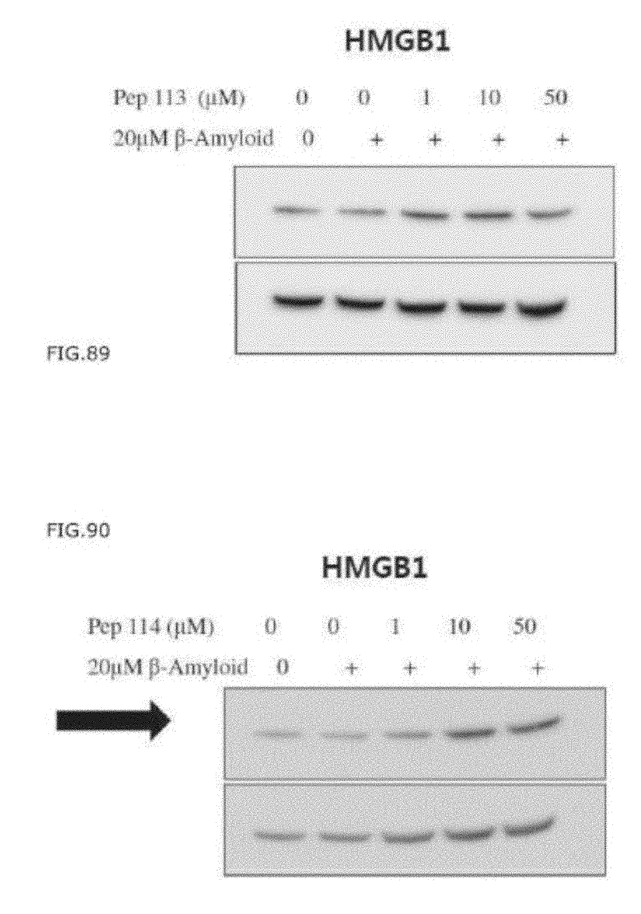

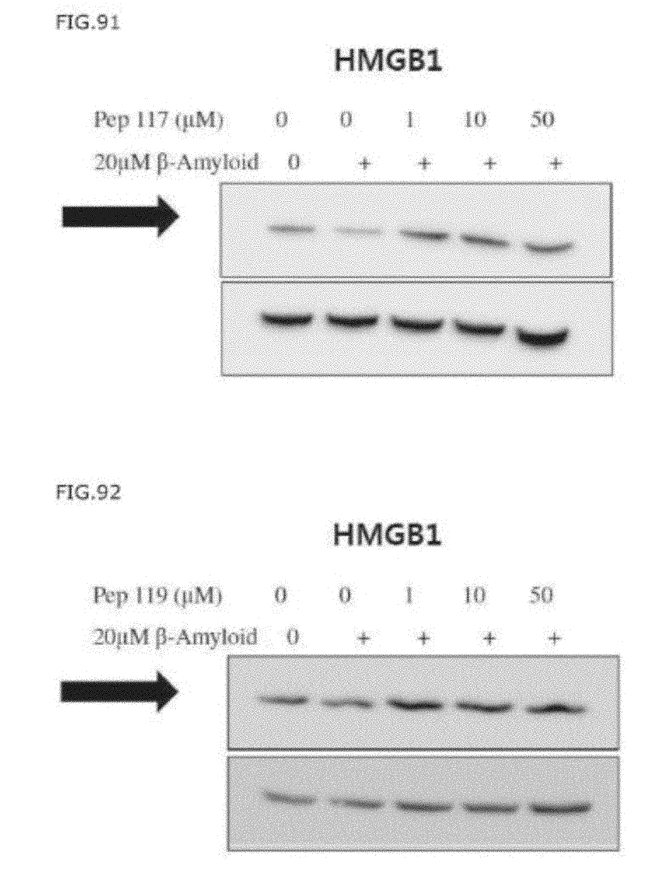

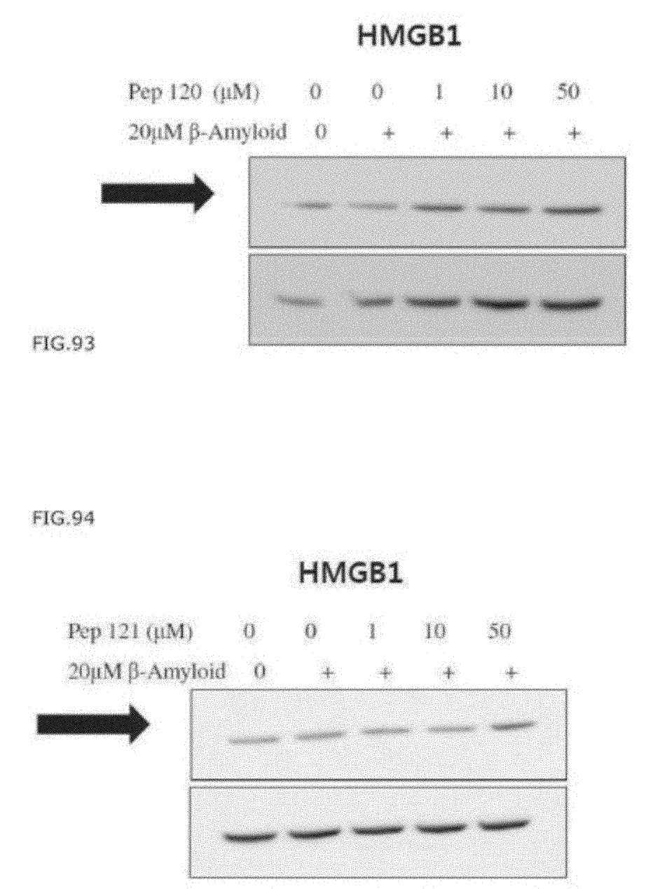

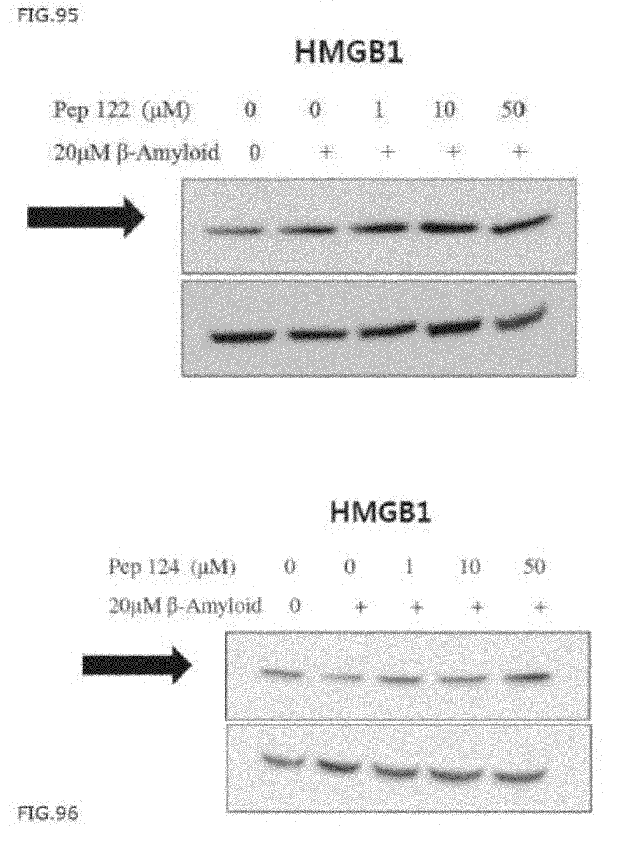

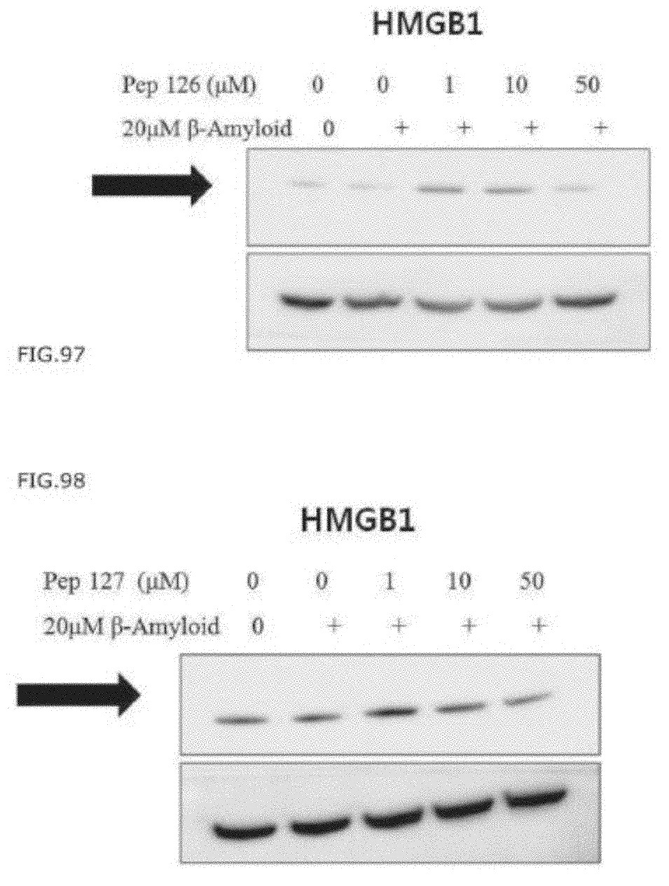

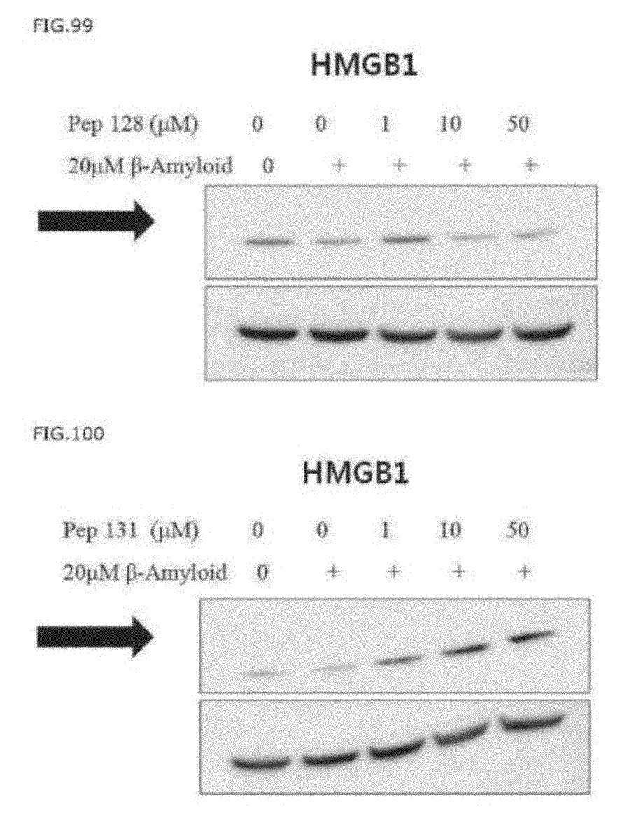

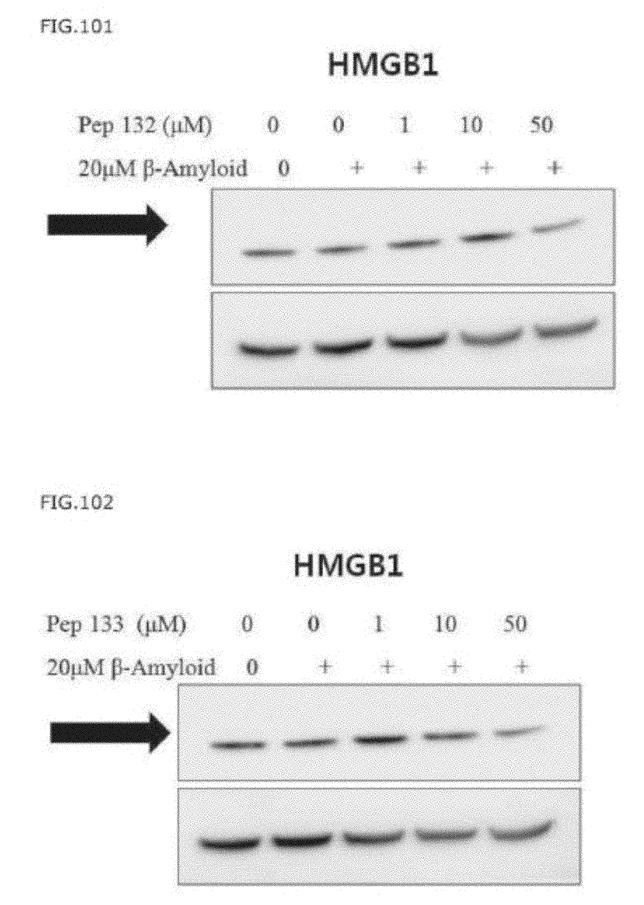

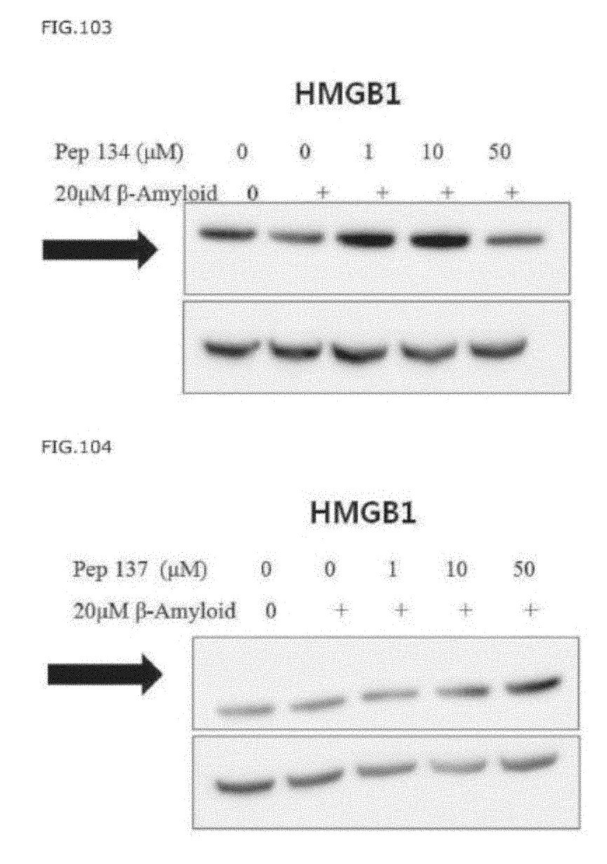

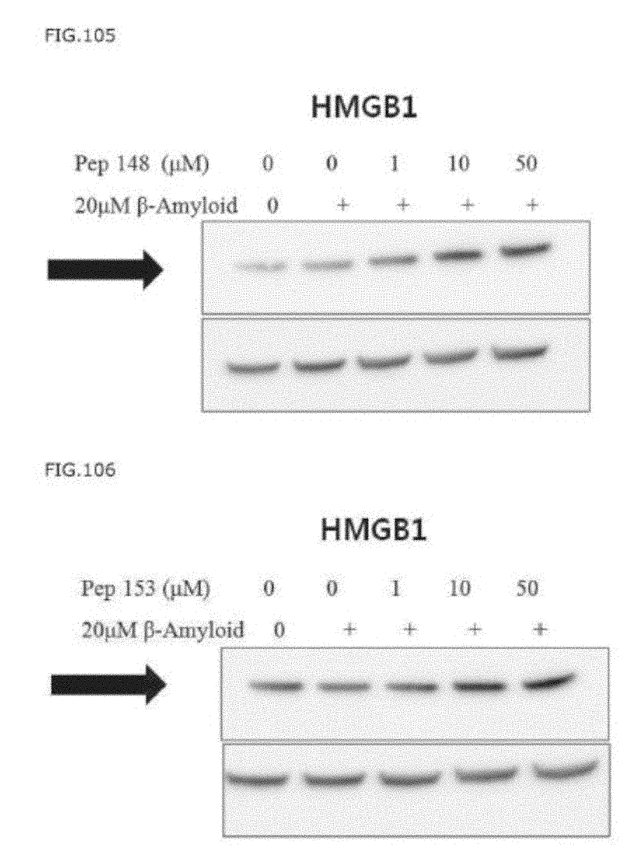

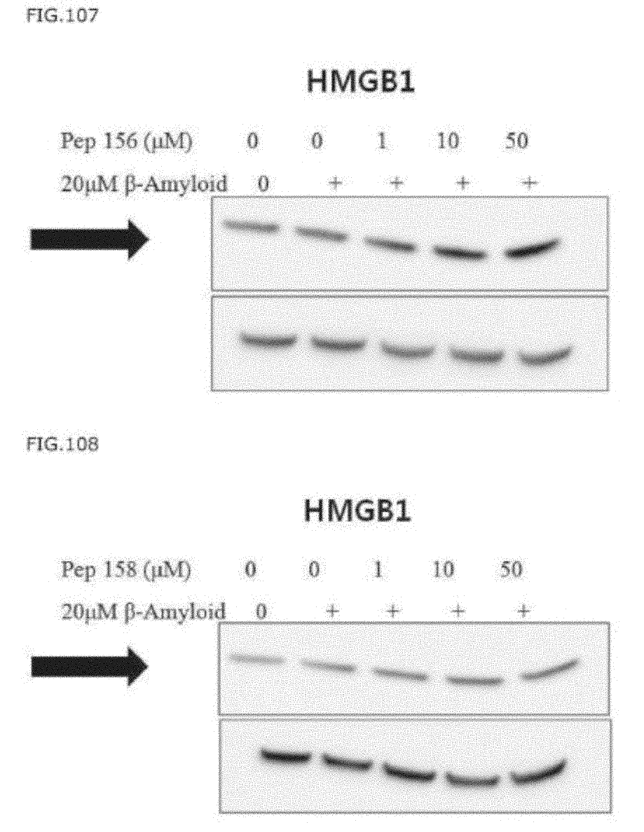

As a result of the western blot analysis, peptides showing accumulation of HMGB1 in the cell were selected. FIG. 36 to FIG. 108 are the western blot results of selected peptides. Tubulins in these figures are used for confirming protein expression. The sequences of the selected peptides are as follows:

SEQ ID NO: 1, SEQ ID NO: 2, SEQ ID NO: 4, SEQ ID NO: 6, SEQ ID NO: 7, SEQ ID NO: 8, SEQ ID NO: 9, SEQ ID NO: 10, SEQ ID NO: 12, SEQ ID NO: 14, SEQ ID NO: 15, SEQ ID NO: 17, SEQ ID NO: 18, SEQ ID NO: 20, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 44, SEQ ID NO: 45, SEQ ID NO: 52, SEQ ID NO: 57, SEQ ID NO: 60, SEQ ID NO: 61, SEQ ID NO: 62, SEQ ID NO: 64, SEQ ID NO: 65, SEQ ID NO: 67, SEQ ID NO: 68, SEQ ID NO: 69, SEQ ID NO: 91, SEQ ID NO: 99, SEQ ID NO: 100, SEQ ID NO: 104, SEQ ID NO: 106, SEQ ID NO: 107, SEQ ID NO: 108, SEQ ID NO: 109, SEQ ID NO: 111, SEQ ID NO: 112, SEQ ID NO: 115, SEQ ID NO: 117, SEQ ID NO: 118, SEQ ID NO: 119, SEQ ID NO: 120, SEQ ID NO: 122, SEQ ID NO: 124, SEQ ID NO: 125, SEQ ID NO: 126, SEQ ID NO: 129, SEQ ID NO: 130, SEQ 30 ID NO: 131, SEQ ID NO: 132, SEQ ID NO: 135, SEQ ID NO: 146, SEQ ID NO: 151, SEQ ID NO: 154, and SEQ ID NO: 156.

### Example 4

### Preparation of pep(CPP)-FITC Conjugate

(1) Preparation of pep(CPP)-FITC Conjugate

A conjugate of the peptides with SEQ ID NO:71, SEQ ID NO:82, SEQ ID NO:96, SEQ ID NO:142, SEQ ID NO:153, and SEQ ID NO:164 combined with FITC was manufactured as follows, for example, a conjugate of pep1 with SEQ ID NO: 165 and FITC, in other words, FITC-linker-pep1 was manufactured as follows.

The basic framework of peptide, NH2-linker-E(OtBu)-A-R(Pbt)-P-A-L-L-T(tBu)-S(tBu)-R(Pbt)L-R(Pbt)-F-I-P-K(Boc)-2-chloro-Trityl Resin) which was obtained according to the manufacturing methods described in Example 1, was reacted with FITC. Specifically, FITC (fluorescein-5isothiocyanate) (8 equivalent) and DIPEA (N,N-Diisopropylethylamine) (16 equivalent) were melted in DMF. The DMF solution was added, and reacted at room temperature for 2 hours, then washed sequentially with DMF, MeOH and DMF. As a result, FITC-linker-E(OtBu)-A-R(Pbf)-P-A-L-L-T(tBu)-S(tBu)-R(Pbf)L-R(Pbf)-F-I-P-K(Boc)-2-chloro-Trityl Resin was obtained. The linker herein is 6-aminohexanoic acid, Ahx. TFA/TIS/H20=95/2.5/2.5 was added to the peptide made on the resin, and the conjugate was separated from the resin. A pre-chilled diethyl ether was added to the obtained mixture, and centrifugation was used to precipitate the peptide conjugates. After purification by Prep-HPLC, purity was confirmed with the analytical HPLC and the molecular weight was determined by LC/MS. The peptide synthesized as described above was verified as HTC-pep1 by confirmation of the molecular weight by LC/MS. Then the conjugates were lyophilized.

(2) Preparation of a CPP-FITC Conjugate

The basic framework of the peptide, (NH2-E(OtBu)-A-R(Pbf)-P-A-L-L-T(tBu)-S(tBu)-R(Pbf)L-R(Pbf)-F-I-P-K(Dde)-2-chloro-Trityl Resin) was generated according to the manufacturing methods described in the Example 2-1. (1). To selectively introduce FITC to the C-term of the peptide, the N-term of the peptide was protected from Boc. Then, the Di-tert-butyl dicarbonate (30 equivalent) and DIPEA (30 equivalent) were melted in DMF. The DMF solution was added to the peptide and incubated at room temperature for 2 hours, and the peptide was washed sequentially with DMF, MeOH, and DMF. As a result, Boc-E(OtBu)-A-R(Pbf)-P-A-L-L-T(tBu)-S(tBu)-R(Pbf)L-R(Pbf)-F-I-P-K(Dde)-2-chloro-Trityl Resin was obtained. Hydrazine in 2% of DMF was used to remove Dde which is the protecting group of the C-terminal residue Lys in order to add FITC to the C-terminal of Lys. Then, FITC (8 equivalent) and DIPEA (16 equivalent) were melted in DMF which was added to the peptide reaction mixture, and the mixture was incubated at room temperature for 2 hours, then washed sequentially with DMF, MeOH, DrvfF. As a result, Boc-E(OtBu)-A-R(Pbf)-P-A-L-L-T(tBu)-S(tBu)-R(Pbf)L-R(Pbf) F-I P-K(FITC)-2-chloro-Trityl Resin was obtained. TFA/TIS/H20=95/2.5/2.5 was added to separate the peptide from resin. Pre-chilled diethyl ether was added to the mixture, and centrifugation was used to precipitate the peptides. After purification by Prep-HPLC, purity was confirmed with the analytical HPLC and the molecular weight was confirmed with LC/MS. The obtained substances were verified as pep1-FITC by confirmation of the molecular weight by LC/MS.

The fusion protein for the peptides of SEQ ID NO:71, SEQ ID NO:82, SEQ ID NO:96, SEQ ID NO:142, SEQ ID NO:153, and SEQ ID NO:164 and enhanced green fluorescent protein (enhanced green fluorescent protein, EGFP) (SEQ ID NO: 168, SEQ ID NO: 169) were manufactured as follows:

(1) Preparation of pET21a-EGFP-His•tag Recombinant DNA Clones

In order to cloned pEGFP-N1 vector as the template enhanced green fluorescent protein (enhanced green fluorescent protein—EGFP) into pET21a(+) vector (Novagen) with contain a restriction endonucleases to produce a primer base sequence shown in Table 6.

TABLE 6

| Sequence | | | |
|---|---|---|---|
| ID | | | Length |
| Number | Primer | Sequence | (bp) |
| 11 | EGFP-F | ATATACATAAATTCATGGTGAGCTAACGG | 34 |
| | | CGAGGA | |
| 12 | EGFP-R | GTGCTCGAGCTTGTACAGCTCGTCCAT | 39 |
| | | GCCGAGAGTGAT | |

EGFPF (forward) primer contained restriction sites of Ndel and EcoRI, and which was designed to be added to 20 number of EGFP sequences, EGFP-R (reverse) primer contained restriction sites of XhoII and which was designed to be added to 30 number of EGFP sequences.

In order to gene amplification, Added 100 pmol primer and 2.5 U Taq DNA polymerase added into 50 μl Tris hydrogen chloride (Tris-HCl, pH 9.0) supplemented with 50 mM potassium chloride, 0.1% triton X-100, 1.5 mM magnesium chloride and 150 μM for four types of Deoxyribonucleotide triphosphate (ATP, dTTP, dGTP, dCTP). Then PCR was performed using the 10 ng pEGFP plasmid DNA as a template as follows; denaturation for pEGFP plasmid DNA at 95° C. for 5 minutes, temperature change at 95° C. for 30 seconds, at 46° C. for 30 seconds and at 72° C. for 45 seconds, 30 cycles from performing a polymerase chain reaction and the amplification was confirmed by agarose gel electrophoresis the products.

Ndel and XhoI restriction endonucleases were cloned which were present in multiple cloning site (multiple cloning site, MCS) of Pet21a.

(2) Preparation of pET21a-pep(CPP)-EGFP-His•tag Recombinant DNA Clones

To obtain pep(CPP)-EGFP-His•tag fusion protein of SEQ ID NO:71, SEQ ID NO:82, SEQ ID NO:96, SEQ ID NO:142, SEQ ID NO:153, and SEQ ID NO: 164 constructed primer sequences as Table 7.

TABLE 7

| Sequence | | | Length |
|---|---|---|---|
| Number | Primer | Sequence | (bp) |
| 13 | pep73-F | TATGGTGTATGGCTTTGTGC | 65 |
| | | GCGCGTGCCTGCGCCGCCTG | |
| | | GTGCCGCCGGGCCTGTGGGG | |
| | | CAGCG | |
| 14 | pep73-R | AATTCGCTGCCCCACAGGCC | 67 |
| | | CGGCGGCACCAGGCGGCGCA | |
| | | GGCACGCGCGCACAAAGCCA | |
| | | TACACCA | |
| 15 | pep84-F | TATGCTGGGCCTGGATGATA | 65 |
| | | TTCATCGCGCGTGGCGCACC | |
| | | TTTGTGCTGCGCGTGCGCGC | |
| | | GCAGG | |
| 16 | pep84-R | AATTCCTGCGCGCGCACGCG | 57 |
| | | CAGCACAAAGGTGCGCCACG | |
| | | CGCGATGAATATCATCCAGG | |
| | | CCCAGCA | |
| 17 | pep98-F | TATGAACCGCGGCTTTAAAG | 65 |
| | | CGGGCCGCAACATGCGCCGC | |
| | | AAACTGTTTGGCGTGCTGCG | |
| | | CCTGG | |
| 18 | pep98-R | AATTCCAGGCGCAGCACGCC | 67 |
| | | AAACAGTTTGCGGCGCATGT | |
| | | TGCGGCCCGCTTTAAAGCCG | |
| | | CGGTTCA | |
| 19 | pep144-F | TATGAGCATTATTAAACCGC | 65 |
| | | AGAACACCTATTGCGTGCGC | |
| | | CGCTATGCGGTGGTGCAGAA | |
| | | AGCGG | |
| 20 | pep144_-R | AATTCCGCTTTCTGCACCAC | 67 |
| | | CGCATAGCGGCGCACGCAAT | |
| | | AGGTGTTCTGCGGTTTAATA | |
| | | ATGCTCA | |
| 21 | pep155-F | TATGCGCACCAGCATTCGCG | 65 |
| | | CGAGCCTGACCTTAACCGC | |
| | | GGCTTTAAAGCGGCCGCAA | |
| | | CATGG | |
| 22 | pep155-R | AATTCCATGTTGCGGCCCGC | 67 |
| | | TTAAAGCCGCGGTTAAAGG | |
| | | TCAGGCTCGCGCGAATGCTG | |
| | | GTGCGCA | |
| 23 | pep-RIA-148-F | TATGCTGCTGACCAGCCGCC | 29 |
| | | TGCGCTTTG | |
| 24 | pep-RIA-148-R | AATTCAAAGCGCAGGCGGCT | 31 |
| | | GGTCAGCAGCA | |

Each F (forward) primer contained restriction sites of Ndel, and which was designed to be added to DNA sequencing pep(CPP) of SEQ ID NO:71, SEQ ID NO:82, SEQ ID NO:96, SEQ ID NO:142, SEQ ID NO:153, and SEQ ID NO:164. Each R (reverse) primer contained restriction sites of EcoRI, and which was designed to be added to DNA sequencing pep(CPP) of SEQ ID NO:71, SEQ ID NO:82, SEQ ID NO:96, SEQ ID NO:142, SEQ ID NO:153, and SEQ ID NO:164.

In order to synthesis of oligonucleotide, 10 uM each F-R primers added into 500 μl solution supplemented with 10 mM Tris hydrogen chloride (Tris-HCl, pH 7.5-8.0), 50 mM potassium chloride, 1 mM ethylenediaminetetraacetic acid. Then, PCR was performed as follows; denaturation for primers at 95° C. for 2 minutes, slowly reduced temperature to reaction for at 25° C. for 45 minutes, and storage at 4° C.

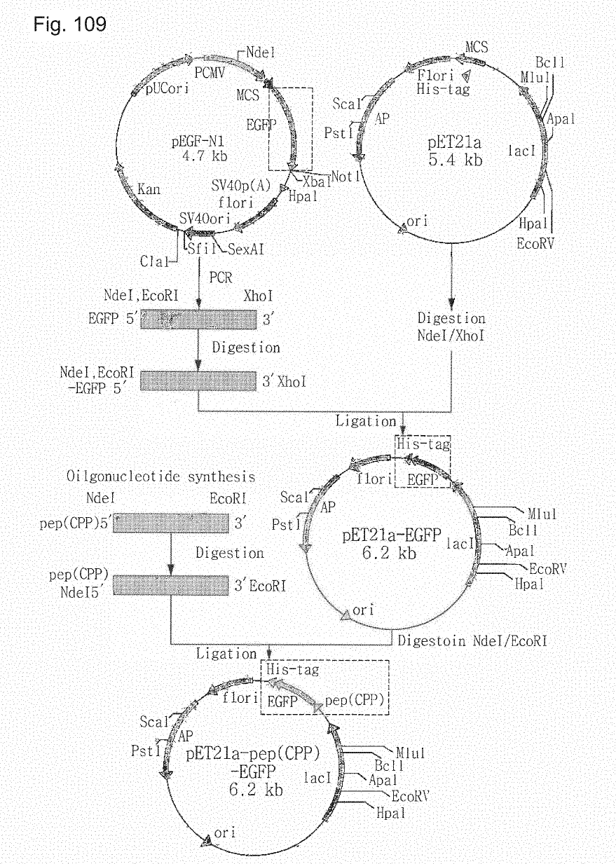

The synthesized oligonucleotide product restricted by Ndel and EcoRI restriction enzymes, and cloned into Ndel and EcoRI restriction sites of pET21a-EGFP-His•tag recombination DNA clones, then constructed pET21 a-pep(CPP)-His•tag recombination DNA clones of SEQ ID NO:71, SEQ ID NO:82, SEQ ID NO:96, SEQ ID NO:142, SEQ ID NO:153, and SEQ ID NO:164 (FIG. 109).

(3) Preparation and Purification of the Fusion Protein

pET21a-EGFP-His•tag recombination DNA clones and pET21a-pep(CPP)-His•tag recombination DNA clones of SEQ ID NO:71, SEQ ID NO:82, SEQ ID NO:96, SEQ ID NO:142, SEQ ID NO:153, and SEQ ID NO:164 were transformed in bacteria, and purified protein. Particularly, transformed using the E. coli BL21(DE3) (Invitrogen, Carlsbad, Calif., USA) and growth in 5 ml LB/ampicillin medium then moved to incubation in 100 ml medium. For this case, added 50 ng/ml ampicillin ratios. Continually, stirred culture for 2 to 3 hours at 37° C. and measured the absorbance to growth until 0.6 to 0.8 ranges. In order to expression of fusion protein, treated 0.1 mM1 mM IPTG (Isopropyl (β-D-1-thiogalactopyranoside) and the cells were additionally cultured for 3 to 16 hours at 16° C. to 37° C. and then centrifuged for 5 minutes at 5000 rpm.

The result for centrifuged of expressed protein, Visually confirm that the cells can take on a green light when overexpressed. Also, His•tag purification kit, Ni-TAT protein isolation kit (Prod, #31314, QuiagenUSA) to be separated as indicated by the manufacturer.

### Example 5: Experimental Cell Penetration of Pep(CPP)-FITC Conjugate

(1) Experimental Cell Penetration in HeLa Cell Line—Cell Culture

Homo sapiens cervix adenocarcinoma cell line as HeLa cell lines were purchased from ATCC (American Type Cell Culture). The cells were cultured in MEM supplemented with 10°/o fetal bovine serum (Invitrogen, 1JSA), Earle's salts, non-essential amino acids, sodium pyruvate and 100 g/ml penicillin and 10 units/ml streptomycin and cultured at 37° C., 5% CO₂ incubator.

Flow Cytometry and Confocal Microscopy Analysis of Cell Penetrating

Flow cytometry and Confocal microscope analysis were performed to compare the between degree of cellular uptake of the cells were treated with SEQ ID NO:71, SEQ ID NO:82, SEQ ID NO:96, SEQ ID NO:142, SEQ ID NO:153, and SEQ ID NO:164, pep (CPP) and control.

The cell line was divided in a 6-well plate and cultured in a medium containing 10% fetal bovine serum (Invitrogen, USA), 100 μg/ml penicillin, 100 units/ml streptomycin at 37° C., 5% CO₂ incubator for 12 hours. After washing the cell line with PBS, starvation was induced in a Minimum Essential Medium for an hour. 20 μM of each carrier peptide was treated and cultured at 37° C. for an hour. After repeating the step of washing the cells with PBS for three times, Trypsin-EDTA was treated form 10 mins at 37° C. to separate the carrier peptide on the outside of the cell. cells were collected with refrigerated PBS and centrifugation was performed to repeat the step of washing the cells for three times. After then, the cells were suspended in 0.5 ml of PBS containing 4% Paraformaldehyde and fluorescence of the cells was analyzed using FACS Calibur (Becton Dickinson). The cellular uptake aspect of control and various peptides combined with FITC was compared and analyzed by WI (Mean Fluorescence Intensity).

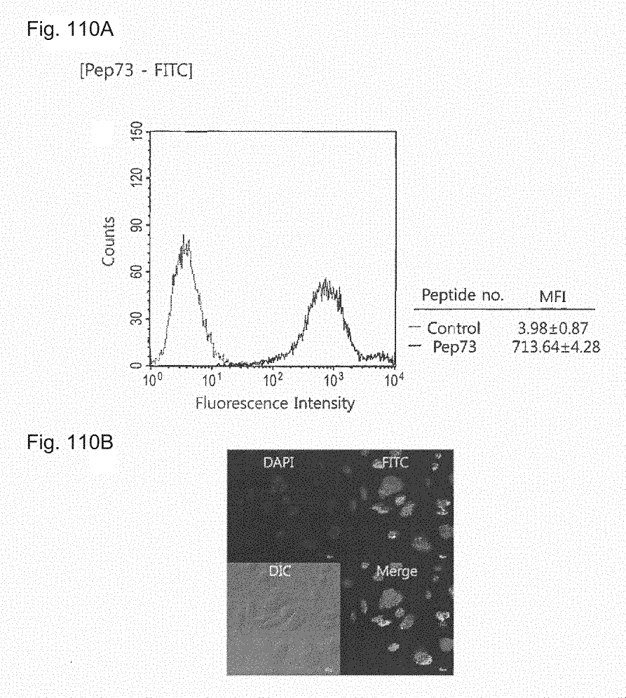

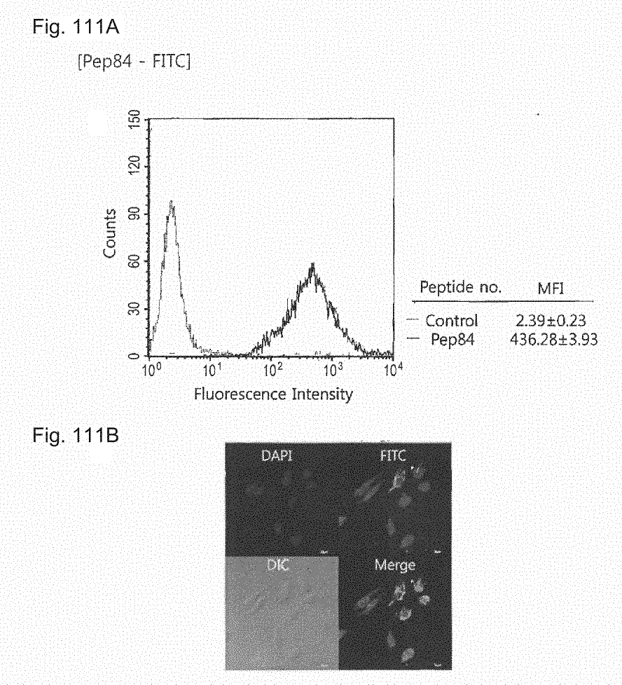

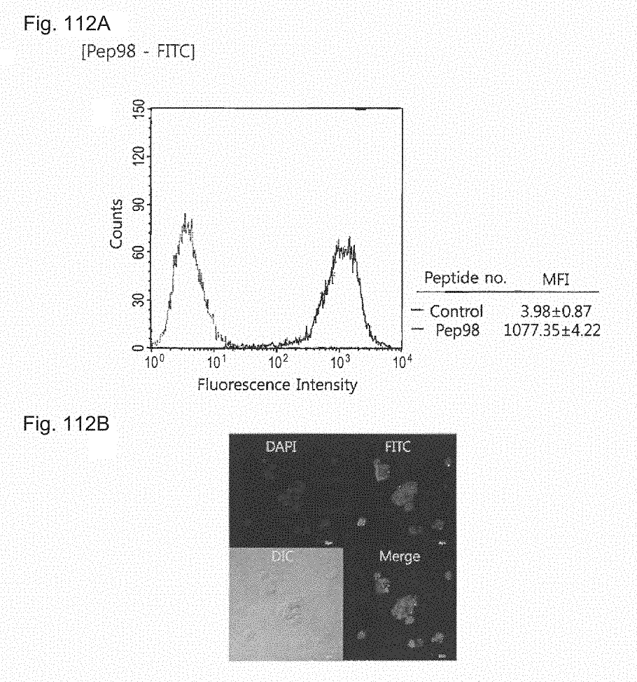

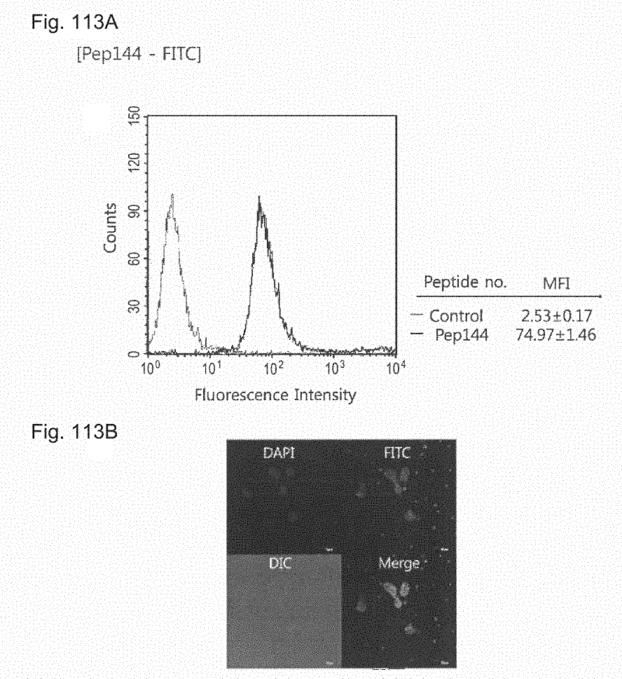

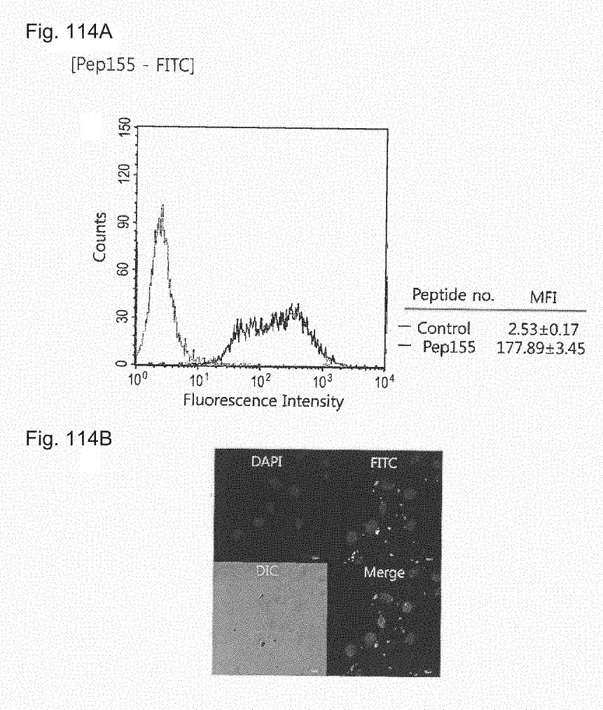

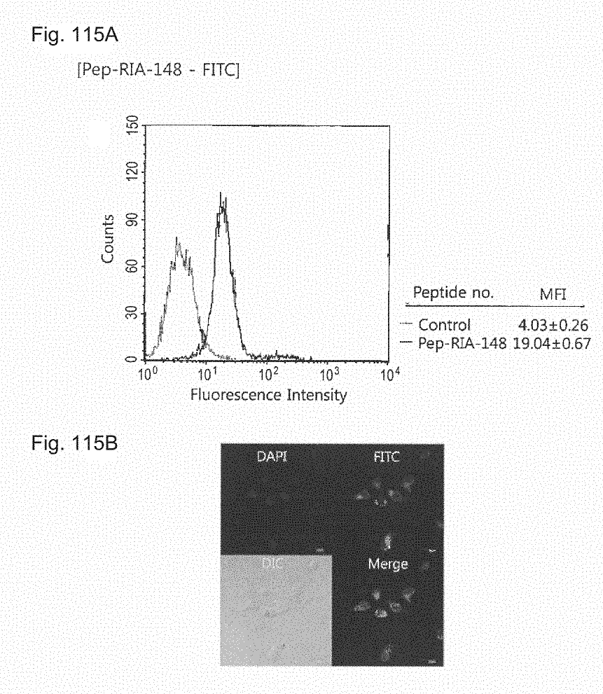

The cultured cell line was divided in chamber well and cultured in a medium containing 10% fetal bovine serum (Invitrogen, USA), 100 μg/ml penicillin and 100 units/ml streptomycin at 37° C., 5% CO₂ incubator for 12 hours. After washing the cells with PBS, starvation was induced in Minimum Essential Medium an hour. 10 μM of each peptide was treated and cultured at 37° C. for an hour. After repeating the step of washing the cells with PBS for 3 times, cells were fixed in room temperature for 15 mins by 2% (v/v) Paraformaldehyde. The nucleus was dyed with DAPI (4′,6-diamldino-2-phenyllndole) in room temperature, and cells were compared and analyzed by Confocal microscope analysis. The result is same as shown in FIG. 110A to FIG. 115B.

(2) Cell Penetrating Property in Huh7 Cell Line

Cell Culture

Human hepatocellular carcinoma cell line as Huh7 lines were purchased from ATCC (American Type Cell Culture) and used as suspended cells. The cells were cultured in MEM supplemented with 10% fetal bovine serum (Invitrogen, USA), Earle's salts. non-essential amino acids, sodium pyruvate and 100 g/ml penicillin and 10 units/ml streptomycin and cultured at 37° C., 5% CO₂ incubator.

The Screening Analysis of Cell Penetrating Used by Flow Cytometry

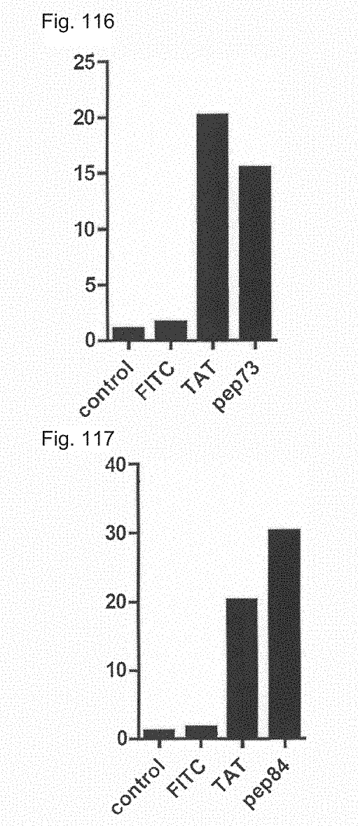

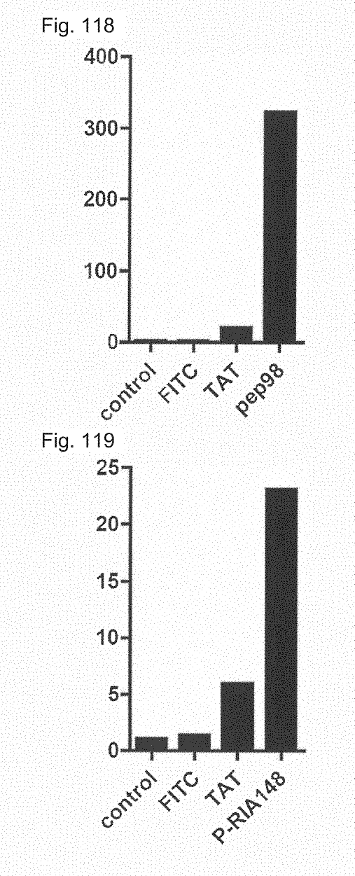

To confirm the cell penetrating of peptides, the Huh7 cell lines were treated with SEQ ID NO:71, SEQ ID NO:82, SEQ ID NO:96, SEQ ID NO:142, SEQ ID NO:153, and SEQ ID NO:164 and analyzed by Flow cytometry. The analyzed method. was also confirmed by the same manner as described above example (1) in HeLa cell lines. Also, the result showed in FIG. 116 to FIG. 119.

(3) Experimental Cell Penetration in Human T Lymphocyte Cell Lines

Cell Culture

Human T-cell leukemia cell line as Jurket was purchased from ATCC (American Type Cell Culture) and used as suspended cells. The cells were cultured in RPMI 1640 supplemented with 10% fetal bovine serum (Invitrogen, USA), Earle's salts, non-essential amino acids, sodium pyruvate and 100 μg/ml penicillin and 10 units/ml streptomycin and cultured at 37° C., 5% CO₂ incubator. Human derived lymphocytes (lymphocyte) separated from the healthy human blood (50 ml) and then collected layer of peripheral blood mononuclear cells (PBMC) and lymphocytes used by Biocoll separating solution (Biochrom AG, Berlin, Germany).

The Screening Analysis of Cell Penetrating Used by Flow Cytometry

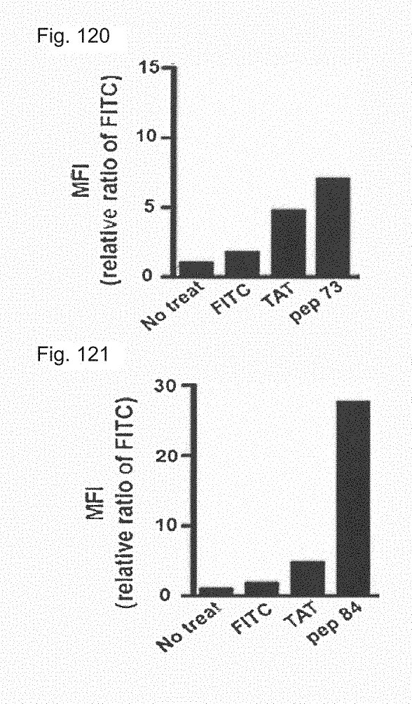

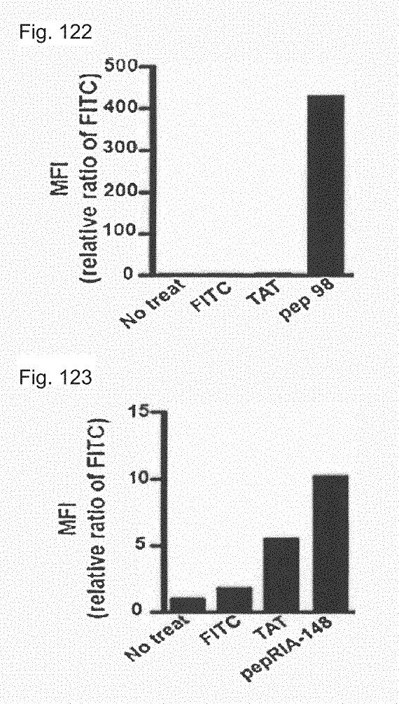

To confirm the cell penetrating of peptides, the human T-cell leukemia cell lines were treated with SEQ ID NO:71, SEQ ID NO:82, SEQ ID NO:96, SEQ ID NO:142, SEQ ID NO:153, and SEQ ID NO:164 and analyzed by Flow cytometry. The analyzed method was also confirmed by the same manner as described above example (1) in HeLa cell lines. Also, the result showed in FIG. 120 to FIG. 123.

(4) Analysis of Cell Viability and Toxicity

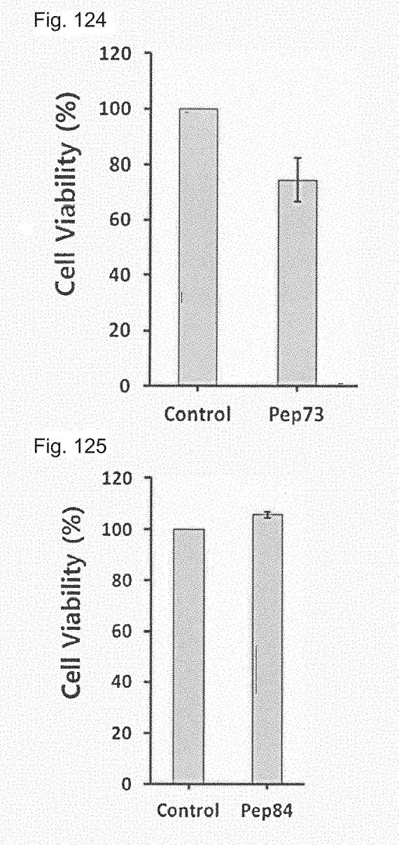

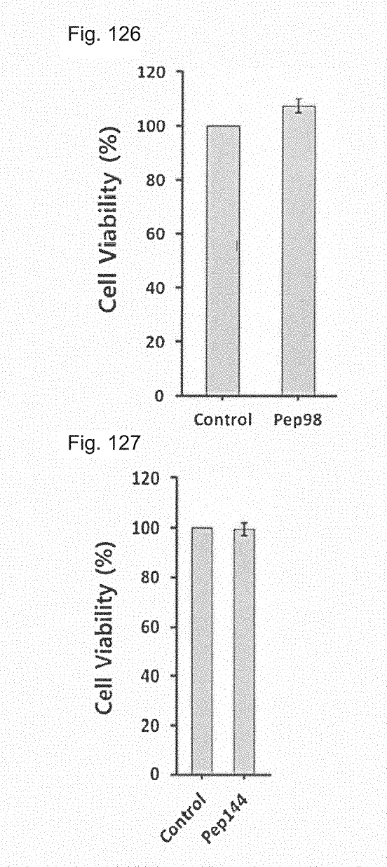

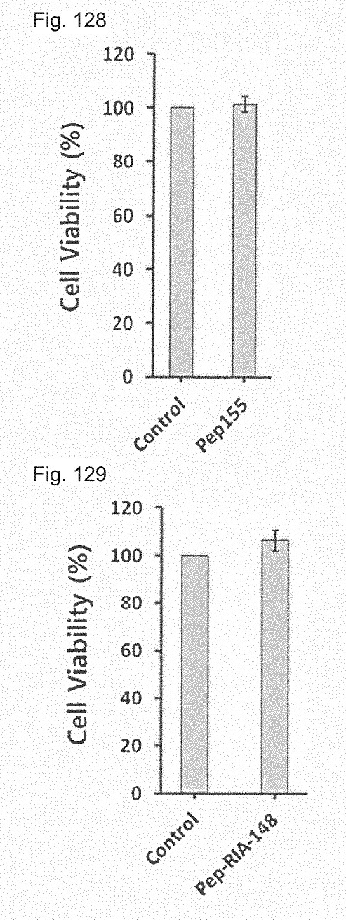

The HeLa cell lines were cultured by the same manner as described above, which divided into 96-well plate and cultured in a medium containing 10% fetal bovine serum (Invitrogen, USA), 100 μg/ml penicillin, 100 units/ml streptomycin at 37° C., 5% CO₂ incubator for 12 hours. After washing the cell line with PBS, starvation was induced in a Minimum Essential Medium for an hour. 20 uM of each carrier peptide was treated and cultured at 37° C. for an hour. After cultured cells, analyzed cell viability and toxicity used by MTT assay. The results showed in FIG. 124 to FIG. 129.

### Example 6: Experimental Cell Penetration of Pep(CPP)-EGFP Fusion Protein

Cell Culture

Homo sapiens cervix adenocarcinoma cell line as HeLa cell lines were purchased from ATCC (American Type Cell Culture). The cells were cultured in MEM supplemented with 10% fetal bovine serum (Invitrogen, USA), Earle's salts, non-essential amino acids, sodium pyruvate and 100 HeLa cell lines were purchased from ATCC (Am and cultured at 37° C., 5% CO₂ incubator.

Flow Cytometry and Confocal Microscopy Analysis of Cell Penetrating

The reporter gene as enhanced green fluorescent protein (enhanced green fluorescent protein, EGFP) was combined with the peptides of SEQ ID NO:71, SEQ ID NO:82, SEQ ID NO:96, SEQ ID NO:142, SEQ ID NO:153, and SEQ ID NO:164 to prepared to fusion protein and treated into cell lines. Flow cytometry and Confocal microscope analysis were performed to compare the between degree of cellular uptake of the cells were treated with pep(CPP)-EGFP fusion protein and EGFP protein.

The cell line was divided into 12-plate and cultured in a medium containing 10% fetal bovine serum (Invitrogen, USA), 100 μg/ml penicillin, 100 units/ml streptomycin at 37° C., 5% CO2 incubator for 12 hours. After washing the cell line with PBS, starvation was induced in a Minimum Essential Medium for an hour. 20 uM of each carrier peptide was treated and cultured at 37° C. for an hour. The cells were washed by PBS for three times, treated Trypsin-EDTA at 37° C. for an hour. The removed pep(CPP)-EGFP fusion protein from the cell outside. Collected cells used by frozen PBS and washed three times by centrifused. After centrifused, then used 0.5 ml PBS containing 4% Paraformaldehyde to suspended cells and analysed FACS Calibur (Becton Dickinson), MFI (mean fluorescence intensity) was performed to compare the between degree of cellular uptake of the cells were treated with pep(CPP)-EGFP fusion protein and EGFP protein.

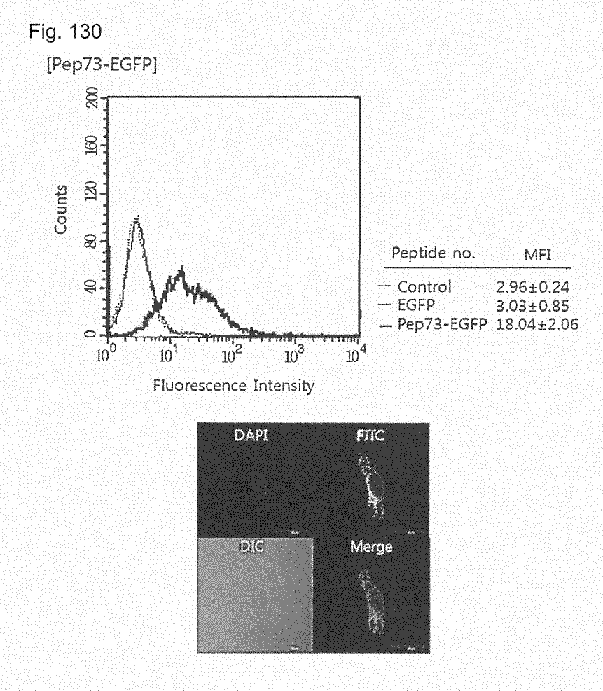

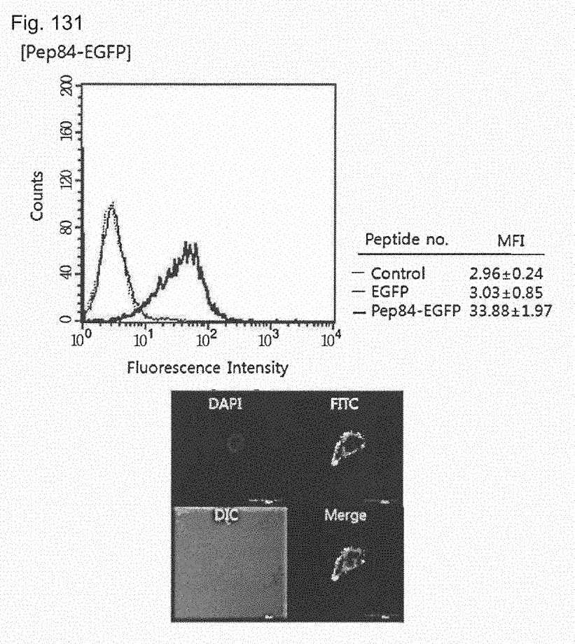

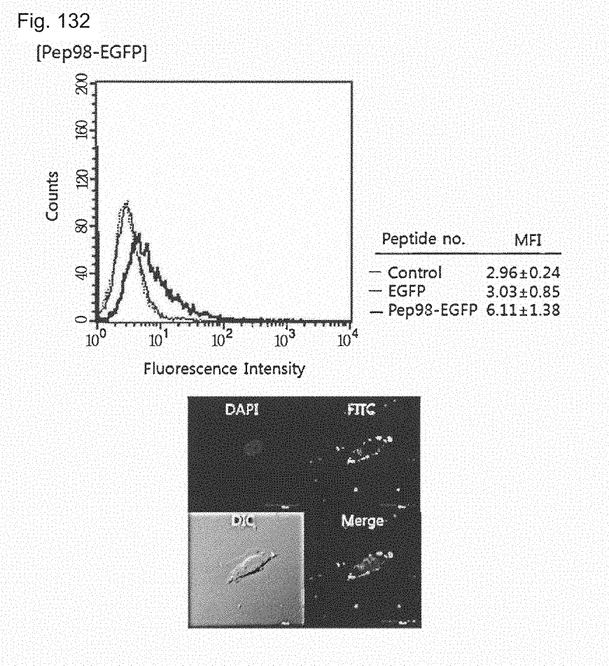

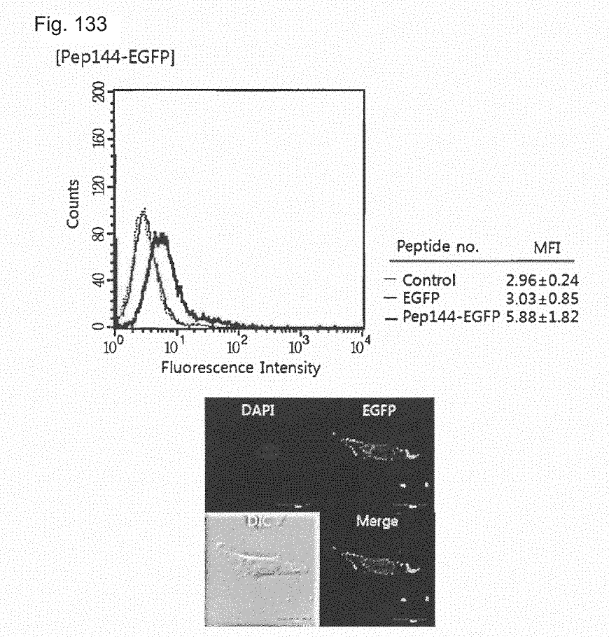

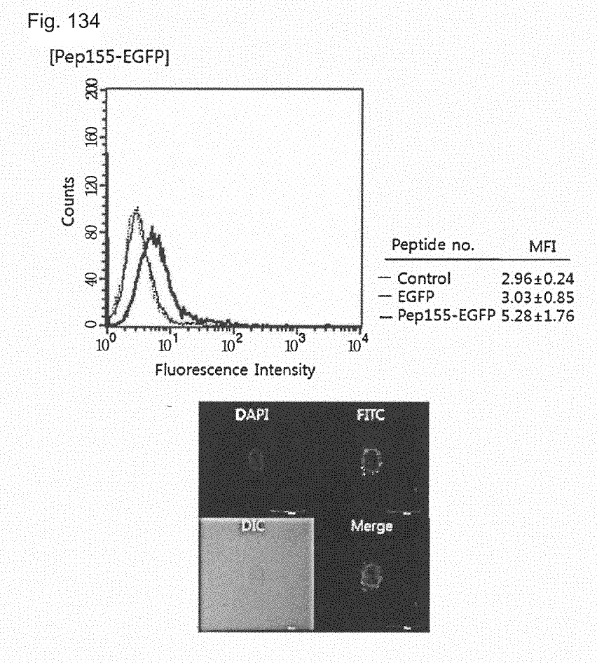

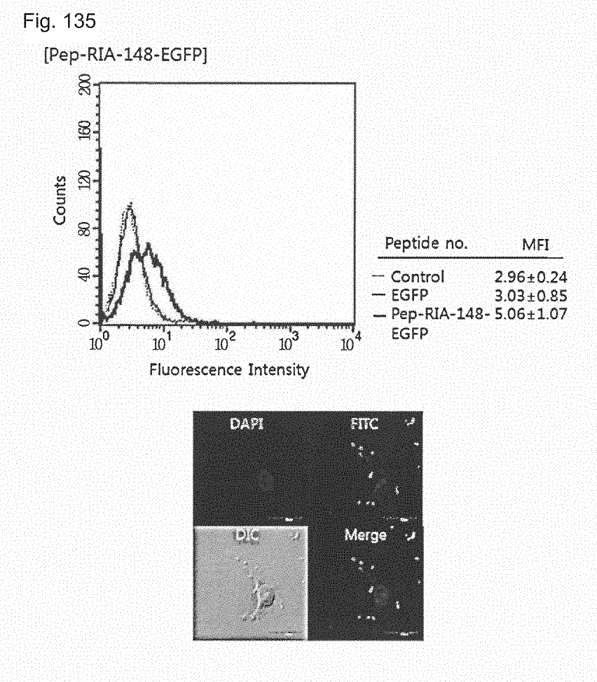

The above cultured cell lines divided into chamber well and cultured in a medium containing 10% fetal bovine serum (Invitrogen, USA), 100 ng/ml penicillin, 100 units/ml streptomycin at 37° C., 5% CO₂ incubator for 12 hours. After washing the cell line with PBS. starvation was induced in a Minimum Essential Medium for two hours. The cells were washed by PBS for three times and fixed with 2% (v/v) Paraformaldehyde at room temperature for 15 minutes. After fixed, used DAPI (4′,6-diarnidino-2-phenylinclole) to dyed nucleus at room temperature and analyzed by confocal microscope. The results showed in FIG. 130 to FIG. 135.

1-21. (canceled)

22. A cell-penetrating peptide comprising:
(a) SEQ ID NO: 153; or
(b) an amino acid sequence comprising at least 80% sequence identity to SEQ ID NO:153; or
(c) a fragment of an amino acid sequence defined in (a) or (b).

23. A composition comprising the cell-penetrating peptide of claim 22.

24. A conjugate comprising an isolated carrier peptide and an active ingredient, wherein the carrier peptide is 20 amino acids in length and consists of SEQ ID NO:153.

25. The conjugate of claim 24, wherein the isolated carrier peptide and the active ingredient are joined by a linker.

26. The conjugate of claim 24, wherein the isolated carrier peptide and the active ingredient are joined via a non-covalent bond.

27. The conjugate of claim 24, wherein the active ingredient is at least one selected from the group consisting of a protein, a nucleic acid, a peptide, lipid, a glycol-lipid, a mineral, a sugar, a nano-particle, a biological product, a contrast agent, a drug, and a chemical compound.

28. The conjugate of claim 24, wherein the active ingredient is fluorescein isothiocyanate (FITC) or Green Fluorescent Protein (GFP).

29. The conjugate of claim 27, wherein the contrast agent is selected from the group consisting of a radiopaque contrast agent, a paramagnetic contrast agent, a superparamagnetic contrast agent, and a CT contrast agent.

30. The conjugate of claim 29, wherein the contrast agent comprises iron.

31. The conjugate of claim 29, wherein the contrast agent comprises ferrocenecarboxylic acid.

32. A composition comprising the conjugate according to claim 24 as an active ingredient.

33. The composition of claim 32, comprising 0.1 μg/mg to 1.0 mg/mg of the isolated carrier peptide.

34. The composition of claim 32, wherein the composition is a pharmaceutical composition formulated for oral, rectal, transdermal, intravenous, intramuscular, intraperitoneal, in the bone marrow, epidural, or subcutaneous administration.

35. The composition of claim 32, wherein the composition is a cosmetic composition.

36. The composition of claim 32, wherein the composition is a health food composition.

37. A method for treating or preventing a mitochondrial related disease or disorder by administering the composition according to claim 32.


SEQUENCE LISTING (182 sequences)

SEQ ID NO: 1 - protein, 30 aa, Homo sapiens
ALFSVLNYERARRPGLLGASVLGLDDIHRA

SEQ ID NO: 2 - protein, 15 aa, Homo sapiens
SVLNYERARRPGLLG

SEQ ID NO: 3 - protein, 10 aa, Homo sapiens
GLLGASVLGL

SEQ ID NO: 4 - protein, 10 aa, Homo sapiens
ALLTSRLRFI

SEQ ID NO: 5 - protein, 9 aa, Homo sapiens
RPALLTSRL

SEQ ID NO: 6 - protein, 9 aa, Homo sapiens
RLTSRVKAL

SEQ ID NO: 7 - protein, 15 aa, Homo sapiens
RTFVLRVRAQDPPPE

SEQ ID NO: 8 - protein, 15 aa, Homo sapiens
RLTSRVKALFSVLNY

SEQ ID NO: 9 - protein, 15 aa, Homo sapiens
AERLTSRVKALFSVL

SEQ ID NO: 10 - protein, 9 aa, Homo sapiens
YERARRPGL

SEQ ID NO: 11 - protein, 9 aa, Homo sapiens
LLGASVLGL

SEQ ID NO: 12 - protein, 10 aa, Homo sapiens
VLGLDDIHRA

SEQ ID NO: 13 - protein, 10 aa, Homo sapiens
GASVLGLDDI

SEQ ID NO: 14 - protein, 10 aa, Homo sapiens
ALFSVLNYER

SEQ ID NO: 15 - protein, 10 aa, Homo sapiens
SVLNYERARR

SEQ ID NO: 16 - protein, 10 aa, Homo sapiens
SVLGLDDIHR

SEQ ID NO: 17 - protein, 10 aa, Homo sapiens
FSVLNYERAR

SEQ ID NO: 18 - protein, 10 aa, Homo sapiens
NYERARRPGL

SEQ ID NO: 19 - protein, 10 aa, Homo sapiens
YERARRPGLL

SEQ ID NO: 20 - protein, 10 aa, Homo sapiens
RPGLLGASVL

SEQ ID NO: 21 - protein, 9 aa, Homo sapiens
ERARRPGLL

SEQ ID NO: 22 - protein, 9 aa, Homo sapiens
VLGLDDIHR

SEQ ID NO: 23 - protein, 9 aa, Homo sapiens
SVLNYERAR

SEQ ID NO: 24 - protein, 9 aa, Homo sapiens
VLNYERARR

SEQ ID NO: 25 - protein, 9 aa, Homo sapiens
ARRPGLLGA

SEQ ID NO: 26 - protein, 9 aa, Homo sapiens
PGLLGASVL

SEQ ID NO: 27 - protein, 9 aa, Homo sapiens
RRPGLLGAS

SEQ ID NO: 28 - protein, 9 aa, Homo sapiens
ALFSVLNYE

SEQ ID NO: 29 - protein, 9 aa, Homo sapiens
GLLGASVLG

SEQ ID NO: 30 - protein, 9 aa, Homo sapiens
SVLGLDDIH

SEQ ID NO: 31 - protein, 8 aa, Homo sapiens
ERARRPGL

SEQ ID NO: 32 - protein, 8 aa, Homo sapiens
ARRPGLLG

SEQ ID NO: 33 - protein, 8 aa, Homo sapiens
GLLGASVL

SEQ ID NO: 34 - protein, 8 aa, Homo sapiens
RARRPGLL

SEQ ID NO: 35 - protein, 8 aa, Homo sapiens
LGASVLGL

SEQ ID NO: 36 - protein, 15 aa, Homo sapiens
VTETTFQKNRLFFYR

SEQ ID NO: 37 - protein, 15 aa, Homo sapiens
LFFYRKSVWSKLQSI

SEQ ID NO: 38 - protein, 15 aa, Homo sapiens
KLQSIGIRQHLKRVQ

SEQ ID NO: 39 - protein, 15 aa, Homo sapiens
EAEVRQHREARPALL

SEQ ID NO: 40 - protein, 15 aa, Homo sapiens
RPALLTSRLRFIPKP

SEQ ID NO: 41 - protein, 15 aa, Homo sapiens
FIPKPDGLRPIVNMD

SEQ ID NO: 42 - protein, 15 aa, Homo sapiens
RTFRREKRAERLTSR

SEQ ID NO: 43 - protein, 15 aa, Homo sapiens
LDDIHRAWRTFVLRV

SEQ ID NO: 44 - protein, 15 aa, Homo sapiens
FVLRVRAQDPPPELY

SEQ ID NO: 45 - protein, 15 aa, Homo sapiens
PQDRLTEVIASIIKP

SEQ ID NO: 46 - protein, 15 aa, Homo sapiens
KSVWSKLQSIGIRQH

SEQ ID NO: 47 - protein, 16 aa, Homo sapiens
LKRVQLRELSEAEVRQ

SEQ ID NO: 48 - protein, 20 aa, Homo sapiens
MPRAPRCRAVRSLLRSHYRE

SEQ ID NO: 49 - protein, 20 aa, Homo sapiens
VLPLATFVRRLGPQGWRLVQ

SEQ ID NO: 50 - protein, 20 aa, Homo sapiens
RGDPAAFRALVAQCLVCVPW

SEQ ID NO: 51 - protein, 20 aa, Homo sapiens
DARPPPAAPSFRQVSCLKEL

SEQ ID NO: 52 - protein, 20 aa, Homo sapiens
VARVLQRLCERGAKNVLAFG

SEQ ID NO: 53 - protein, 20 aa, Homo sapiens
FALLDGARGGPPEAFTTSVR

SEQ ID NO: 54 - protein, 20 aa, Homo sapiens
SYLPNTVTDALRGSGAWGLL

SEQ ID NO: 55 - protein, 20 aa, Homo sapiens
LRRVGDDVLVHLLARCALFV

SEQ ID NO: 56 - protein, 20 aa, Homo sapiens
LVAPSCAYQVCGPPLYQLGA

SEQ ID NO: 57 - protein, 20 aa, Homo sapiens
ATQARPPPHASGPRRRLGCE

SEQ ID NO: 58 - protein, 20 aa, Homo sapiens
RAWNHSVREAGVPLGLPAPG

SEQ ID NO: 59 - protein, 20 aa, Homo sapiens
ARRRGGSASRSLPLPKRPRR

SEQ ID NO: 60 - protein, 20 aa, Homo sapiens
GAAPEPERTPVGQGSWAHPG

SEQ ID NO: 61 - protein, 20 aa, Homo sapiens
RTRGPSDRGFCVVSPARPAE

SEQ ID NO: 62 - protein, 20 aa, Homo sapiens
EATSLEGALSGTRHSHPSVG

SEQ ID NO: 63 - protein, 20 aa, Homo sapiens
RQHHAGPPSTSRPPRPWDTP

SEQ ID NO: 64 - protein, 20 aa, Homo sapiens
CPPVYAETKHFLYSSGDKEQ

SEQ ID NO: 65 - protein, 20 aa, Homo sapiens
LRPSFLLSSLRPSLTGARRL

SEQ ID NO: 66 - protein, 20 aa, Homo sapiens
VETIFLGSRPWMPGTPRRLP

SEQ ID NO: 67 - protein, 20 aa, Homo sapiens
RLPQRYWQMRPLFLELLGNH

SEQ ID NO: 68 - protein, 20 aa, Homo sapiens
AQCPYGVLLKTHCPLRAAVT

SEQ ID NO: 69 - protein, 20 aa, Homo sapiens
PAAGVCAREKPQGSVAAPEE

SEQ ID NO: 70 - protein, 20 aa, Homo sapiens
EDTDPRRLVQLLRQHSSPWQ

SEQ ID NO: 71 - protein, 20 aa, Homo sapiens
VYGFVRACLRRLVPPGLWGS

SEQ ID NO: 72 - protein, 20 aa, Homo sapiens
RHNERRFLRNTKKFISLGKH

SEQ ID NO: 73 - protein, 20 aa, Homo sapiens
AKLSLQELTWKMSVRDCAWL

SEQ ID NO: 74 - protein, 20 aa, Homo sapiens
RRSPGVGCVPAAEHRLREEI

SEQ ID NO: 75 - protein, 20 aa, Homo sapiens
LAKFLHWLMSVYVVELLRSF

SEQ ID NO: 76 - protein, 20 aa, Homo sapiens
FYVTETTFQKNRLFFYRKSV

SEQ ID NO: 77 - protein, 20 aa, Homo sapiens
WSKLQSIGIRQHLKRVQLRE

SEQ ID NO: 78 - protein, 20 aa, Homo sapiens
LSEAEVRQHREARPALLTSR

SEQ ID NO: 79 - protein, 20 aa, Homo sapiens
LRFIPKPDGLRPIVNMDYVV

SEQ ID NO: 80 - protein, 20 aa, Homo sapiens
GARTFRREKRAERLTSRVKA

SEQ ID NO: 81 - protein, 20 aa, Homo sapiens
LFSVLNYERARRPGLLGASV

SEQ ID NO: 82 - protein, 20 aa, Homo sapiens
LGLDDIHRAWRTFVLRVRAQ

SEQ ID NO: 83 - protein, 20 aa, Homo sapiens
DPPPELYFVKVDVTGAYDTI

SEQ ID NO: 84 - protein, 20 aa, Homo sapiens
PQDRLTEVIASIIKPQNTYC

SEQ ID NO: 85 - protein, 20 aa, Homo sapiens
VRRYAVVQKAAHGHVRKAFK

SEQ ID NO: 86 - protein, 20 aa, Homo sapiens
SHVSTLTDLQPYMRQFVAHL

SEQ ID NO: 87 - protein, 20 aa, Homo sapiens
QETSPLRDAVVIEQSSSLNE

SEQ ID NO: 88 - protein, 20 aa, Homo sapiens
ASSGLFDVFLRFMCHHAVRI

SEQ ID NO: 89 - protein, 20 aa, Homo sapiens
RGKSYVQCQGIPQGSILSTL

SEQ ID NO: 90 - protein, 20 aa, Homo sapiens
LCSLCYGDMENKLFAGIRRD

SEQ ID NO: 91 - protein, 20 aa, Homo sapiens
GLLLRLVDDFLLVTPHLTHA

SEQ ID NO: 92 - protein, 20 aa, Homo sapiens
KTFLRTLVRGVPEYGCVVNL

SEQ ID NO: 93 - protein, 20 aa, Homo sapiens
RKTVVNFPVEDEALGGTAFV

SEQ ID NO: 94 - protein, 20 aa, Homo sapiens
QMPAHGLFPWCGLLLDTRTL

SEQ ID NO: 95 - protein, 20 aa, Homo sapiens
EVQSDYSSYARTSIRASLTF

SEQ ID NO: 96 - protein, 20 aa, Homo sapiens
NRGFKAGRNMRRKLFGVLRL

SEQ ID NO: 97 - protein, 20 aa, Homo sapiens
KCHSLFLDLQVNSLQTVCTN

SEQ ID NO: 98 - protein, 20 aa, Homo sapiens
IYKILLLQAYRFHACVLQLP

SEQ ID NO: 99 - protein, 20 aa, Homo sapiens
FHQQVWKNPTFFLRVISDTA

SEQ ID NO: 100 - protein, 20 aa, Homo sapiens
SLCYSILKAKNAGMSLGAKG

SEQ ID NO: 101 - protein, 20 aa, Homo sapiens
AAGPLPSEAVQWLCHQAFLL

SEQ ID NO: 102 - protein, 20 aa, Homo sapiens
KLTRHRVTYVPLLGSLRTAQ

SEQ ID NO: 103 - protein, 20 aa, Homo sapiens
TQLSRKLPGTTLTALEAAAN

SEQ ID NO: 104 - protein, 12 aa, Homo sapiens
PALPSDFKTILD

SEQ ID NO: 105 - protein, 10 aa, Homo sapiens
MPRAPRCRAV

SEQ ID NO: 106 - protein, 20 aa, Homo sapiens
RSLLRSHYREVLPLATFVRR

SEQ ID NO: 107 - protein, 20 aa, Homo sapiens
LGPQGWRLVQRGDPAAFRAL

SEQ ID NO: 108 - protein, 20 aa, Homo sapiens
VAQCLVCVPWDARPPPAAPS

SEQ ID NO: 109 - protein, 20 aa, Homo sapiens
FRQVSCLKELVARVLQRLCE

SEQ ID NO: 110 - protein, 20 aa, Homo sapiens
RGAKNVLAFGFALLDGARGG

SEQ ID NO: 111 - protein, 20 aa, Homo sapiens
PPEAFTTSVRSYLPNTVTDA

SEQ ID NO: 112 - protein, 20 aa, Homo sapiens
LRGSGAWGLLLRRVGDDVLV

SEQ ID NO: 113 - protein, 20 aa, Homo sapiens
HLLARCALFVLVAPSCAYQV

SEQ ID NO: 114 - protein, 20 aa, Homo sapiens
CGPPLYQLGAATQARPPPHA

SEQ ID NO: 115 - protein, 20 aa, Homo sapiens
SGPRRRLGCERAWNHSVREA

SEQ ID NO: 116 - protein, 20 aa, Homo sapiens
GVPLGLPAPGARRRGGSASR

SEQ ID NO: 117 - protein, 20 aa, Homo sapiens
SLPLPKRPRRGAAPEPERTP

SEQ ID NO: 118 - protein, 20 aa, Homo sapiens
VGQGSWAHPGRTRGPSDRGF

SEQ ID NO: 119 - protein, 20 aa, Homo sapiens
CVVSPARPAEEATSLEGALS

SEQ ID NO: 120 - protein, 20 aa, Homo sapiens
GTRHSHPSVGRQHHAGPPST

SEQ ID NO: 121 - protein, 20 aa, Homo sapiens
SRPPRPWDTPCPPVYAETKH

SEQ ID NO: 122 - protein, 20 aa, Homo sapiens
FLYSSGDKEQLRPSFLLSSL

SEQ ID NO: 123 - protein, 20 aa, Homo sapiens
RPSLTGARRLVETIFLGSRP

SEQ ID NO: 124 - protein, 20 aa, Homo sapiens
WMPGTPRRLPRLPQRYWQMR

SEQ ID NO: 125 - protein, 20 aa, Homo sapiens
PLFLELLGNHAQCPYGVLLK

SEQ ID NO: 126 - protein, 20 aa, Homo sapiens
THCPLRAAVTPAAGVCAREK

SEQ ID NO: 127 - protein, 20 aa, Homo sapiens
PQGSVAAPEEEDTDPRRLVQ

SEQ ID NO: 128 - protein, 20 aa, Homo sapiens
LLRQHSSPWQVYGFVRACLR

SEQ ID NO: 129 - protein, 20 aa, Homo sapiens
RLVPPGLWGSRHNERRFLRN

SEQ ID NO: 130 - protein, 20 aa, Homo sapiens
TKKFISLGKHAKLSLQELTW

SEQ ID NO: 131 - protein, 20 aa, Homo sapiens
KMSVRDCAWLRRSPGVGCVP

SEQ ID NO: 132 - protein, 20 aa, Homo sapiens
AAEHRLREEILAKFLHWLMS

SEQ ID NO: 133 - protein, 20 aa, Homo sapiens
VYVVELLRSFFYVTETTFQK

SEQ ID NO: 134 - protein, 20 aa, Homo sapiens
NRLFFYRKSVWSKLQSIGIR

SEQ ID NO: 135 - protein, 20 aa, Homo sapiens
QHLKRVQLRELSEAEVRQHR

SEQ ID NO: 136 - protein, 20 aa, Homo sapiens
EARPALLTSRLRFIPKPDGL

SEQ ID NO: 137 - protein, 20 aa, Homo sapiens
RPIVNMDYVVGARTFRREKR

SEQ ID NO: 138 - protein, 20 aa, Homo sapiens
AERLTSRVKALFSVLNYERA

SEQ ID NO: 139 - protein, 20 aa, Homo sapiens
RRPGLLGASVLGLDDIHRAW

SEQ ID NO: 140 - protein, 20 aa, Homo sapiens
RTFVLRVRAQDPPPELYFVK

SEQ ID NO: 141 - protein, 20 aa, Homo sapiens
VDVTGAYDTIPQDRLTEVIA

SEQ ID NO: 142 - protein, 20 aa, Homo sapiens
SIIKPQNTYCVRRYAVVQKA

SEQ ID NO: 143 - protein, 20 aa, Homo sapiens
AHGHVRKAFKSHVSTLTDLQ

SEQ ID NO: 144 - protein, 20 aa, Homo sapiens
PYMRQFVAHLQETSPLRDAV

SEQ ID NO: 145 - protein, 20 aa, Homo sapiens
VIEQSSSLNEASSGLFDVFL

SEQ ID NO: 146 - protein, 20 aa, Homo sapiens
RFMCHHAVRIRGKSYVQCQG

SEQ ID NO: 147 - protein, 20 aa, Homo sapiens
IPQGSILSTLLCSLCYGDME

SEQ ID NO: 148 - protein, 20 aa, Homo sapiens
NKLFAGIRRDGLLLRLVDDF

SEQ ID NO: 149 - protein, 20 aa, Homo sapiens
LLVTPHLTHAKTFLRTLVRG

SEQ ID NO: 150 - protein, 20 aa, Homo sapiens
VPEYGCVVNLRKTVVNFPVE

SEQ ID NO: 151 - protein, 20 aa, Homo sapiens
DEALGGTAFVQMPAHGLFPW

SEQ ID NO: 152 - protein, 20 aa, Homo sapiens
CGLLLDTRTLEVQSDYSSYA

SEQ ID NO: 153 - protein, 20 aa, Homo sapiens
RTSIRASLTFNRGFKAGRNM

SEQ ID NO: 154 - protein, 20 aa, Homo sapiens
RRKLFGVLRLKCHSLFLDLQ

SEQ ID NO: 155 - protein, 20 aa, Homo sapiens
VNSLQTVCTNIYKILLLQAY

SEQ ID NO: 156 - protein, 20 aa, Homo sapiens
RFHACVLQLPFHQQVWKNPT

SEQ ID NO: 157 - protein, 20 aa, Homo sapiens
FFLRVISDTASLCYSILKAK

SEQ ID NO: 158 - protein, 20 aa, Homo sapiens
NAGMSLGAKGAAGPLPSEAV

SEQ ID NO: 159 - protein, 20 aa, Homo sapiens
QWLCHQAFLLKLTRHRVTYV

SEQ ID NO: 160 - protein, 20 aa, Homo sapiens
PLLGSLRTAQTQLSRKLPGT

SEQ ID NO: 161 - protein, 22 aa, Homo sapiens
TLTALEAAANPALPSDFKTILD

SEQ ID NO: 162 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 163 - protein, 16 aa, Homo sapiens
EARPALLTSRLRFIPK

SEQ ID NO: 164 - protein, 8 aa, Homo sapiens
LLTSRLRF

SEQ ID NO: 165 - protein, 16 aa, Homo sapiens
EARPALLTSRLRFIPK

SEQ ID NO: 166 - protein, 1132 aa, Homo sapiens; identical to SEQ ID NO: 162

SEQ ID NO: 167 - protein, 239 aa, Artificial Sequence
  Green Fluorescent Protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 168 - DNA, 717 bp, Artificial Sequence
  Green Fluorescent Protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 169 - DNA, 34 bp, Artificial Sequence
  EGFP-F
atatacataaattcatggtgagcaagggcgagga

SEQ ID NO: 170 - DNA, 39 bp, Artificial Sequence
  EGFP-R
gtgctcgagcttgtacagctcgtccatgccgagagtgat

SEQ ID NO: 171 - DNA, 65 bp, Artificial Sequence
  pep73-F
tatggtgtatggctttgtgcgcgcgtgcctgcgccgcctggtgccgccgggcctgtggggcagcg

SEQ ID NO: 172 - DNA, 67 bp, Artificial Sequence
  pep73-R
aattcgctgccccacaggcccggcggcaccaggcggcgcaggcacgcgcgcacaaagccatacacca

SEQ ID NO: 173 - DNA, 65 bp, Artificial Sequence
  pep84-F
tatgctgggcctggatgatattcatcgcgcgtggcgcacctttgtgctgcgcgtgcgcgcgcagg

SEQ ID NO: 174 - DNA, 67 bp, Artificial Sequence
  pep84-R
aattcctgcgcgcgcacgcgcagcacaaaggtgcgccacgcgcgatgaatatcatccaggcccagca

SEQ ID NO: 175 - DNA, 65 bp, Artificial Sequence
  pep98-F
tatgaaccgcggctttaaagcgggccgcaacatgcgccgcaaactgtttggcgtgctgcgcctgg

SEQ ID NO: 176 - DNA, 67 bp, Artificial Sequence
  pep98-R
aattccaggcgcagcacgccaaacagtttgcggcgcatgttgcggcccgctttaaagccgcggttca

SEQ ID NO: 177 - DNA, 65 bp, Artificial Sequence
  pep144-F
tatgagcattattaaaccgcagaacacctattgcgtgcgccgctatgcggtggtgcagaaagcgg

SEQ ID NO: 178 - DNA, 67 bp, Artificial Sequence
  pep144-R
aattccgctttctgcaccaccgcatagcggcgcacgcaataggtgttctgcggtttaataatgctca

SEQ ID NO: 179 - DNA, 65 bp, Artificial Sequence
  pep155-F
tatgcgcaccagcattcgcgcgagcctgacctttaaccgcggctttaaagcgggccgcaacatgg

SEQ ID NO: 180 - DNA, 67 bp, Artificial Sequence
  pep155-R
aattccatgttgcggcccgctttaaagccgcggttaaaggtcaggctcgcgcgaatgctggtgcgca

SEQ ID NO: 181 - DNA, 29 bp, Artificial Sequence
  pep-RIA-148-F
tatgctgctgaccagccgcctgcgctttg

SEQ ID NO: 182 - DNA, 31 bp, Artificial Sequence
  pep-RIA-148-R
aattcaaagcgcaggcggctggtcagcagca